(19) United States
(12) Patent Application Publication
(10) Pub. No.: US 2007/0212428 A1
(43) Pub. Date: Sep. 13, 2007

(54) METHODS AND COMPOSITIONS FOR TREATING MOOD DISORDER

(75) Inventor: William A. Wittlin, Ithaca, NY (US)

Correspondence Address: BINGHAM MCCUTCHEN LLP, Three Embarcadero Center, San Francisco, CA, 94111-4067, US

(73) Assignee: MOOD MANAGEMENT SCIENCES, INC., Ithaca, NY (US)

(21) Appl. No.: 11/672,012

(22) Filed: Feb. 6, 2007

Related U.S. Application Data
(63) Continuation-in-part of application No. 11/570,035.

(30) Foreign Application Priority Data
Jun. 4, 2004 (US) PCT/US04/17615

(51) Int. Cl. A61K 31/551; A61K 31/5415; A61K 31/165; A61K 31/195

(52) U.S. Cl. 424/722; 514/225.8; 514/220; 514/563; 514/618

(57) ABSTRACT

The invention relates to compositions and methods for treating mood spectrum disorder. The compositions relate to novel combinations and formulations of pharmaceutical compounds for treatment of mood spectrum disorders. The methods provide for use of the compounds provided herein for the treatment of mood spectrum disorders in addition to the use of mood screens for diagnosing a patient.

### CROSS-REFERENCE TO RELATED APPLICATION

This application is a continuation-in-part of U.S. Ser. No. 11/570,035, filed Dec. 4, 2006, which in turn is a U.S. National Stage under 37 USC 371 of PCT Application No. PCT/US2004/017615, filed Jun. 4, 2004, the contents of each of which are hereby incorporated by reference into the present disclosure.

### FIELD OF THE INVENTION

The field of the invention relates to methods and composition for treatment of mood disorders.

### BACKGROUND OF THE INVENTION

Currently, the majority of primary care physicians (“PCPs”) do not feel sufficiently competent to treat mood spectrum disorders like bipolar (manic-depressive) illness. Although millions of mood-disordered patients receive medical treatment from their PCPs, the vast majority of these patients' psychiatric illnesses go undiagnosed, misdiagnosed or under-treated. For example, Major Depressive Disorder (“MDD”) is known to be under-treated in at least 50% of all depressed patients. Even if a PCP recommends a psychiatric referral, most patients will not comply because of the stigma of seeing a psychiatrist. Patients' aversion to psychiatry manifests as both self-stigmatization and as social stigmatization given society's negative stereotypes of the mentally ill.

Medicine's current failure to diagnose early and to preventatively treat mood disorders entails an enormous cost in human suffering and health care dollars. In 1990, the total cost of bipolar disorder in the United States was estimated at 45 billion dollars. Unfortunately, patients' aversion to psychiatry means that mood-disordered patients, if diagnosed at all, are often only diagnosed once they are sufficiently ill to require psychiatric hospitalization. Furthermore, there is an incalculable cost for the thousands of mood-disordered patients who go on to commit suicide. On average, one in five bipolar patients will eventually kill themselves. Additionally, there are 35,000 suicides per year attributed to depressive illness alone.

Current trends in medicine favor greater and greater utilization of PCPs and declining utilization of specialists. Medicare, Medicaid, insurance companies, PPOs, HMOs and managed care companies, all make PCPs gatekeepers to specialty care. Currently, fifty percent of MDDs are under-treated. No more than 50% of patients with major depression actively seek and receive adequate treatment. The response rate in the treatment of MDD is only about fifty percent. Thus, fully half of all depressed patients could benefit from augmentation of their antidepressant treatment.

Already, non-psychiatric physicians are prescribing 95% of all psychotropic medication. A study published in 2002 found that two thirds of all psychiatric patients were treated by PCP's. (Manning et al., “Mood Disorders in Family Practice: Beyond Unipolarity to Bipolarity.” Primary Care Companion, J. Clin. Psychiatry, 4(4):143 (2002)). Furthermore, the National Managed Care Depression Data Base, which contains thirty six million patients, found that only 8.6% of depressed patients were in psychiatric specialty care. (Paxil CR Economic Study Results. Distributed by GlaxoSmithKline, p. 7, Spring, 2004.) In today's medicine, and even more so in our medical future, either PCPs will treat mood disorders or the majority of mood disorders will go untreated. Current data strongly support the fact that every subsequent mood episode may have a cumulative effect, worsening prognosis. The five-year risk of relapse for a bipolar patient into either mania or depression is 73%. Of all those patients who relapse, two thirds experience multiple relapses.

### SUMMARY OF THE INVENTION

Novel combinations of pharmaceutical compositions are provided herein to treat mood spectrum disorder. In addition, methods are also provided for the treatment of mood spectrum disorders. In one embodiment, a composition is provided comprising bupropion and a tricyclic antidepressant formulated into a single dosage formulation. In a further embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline .

In another embodiment, a composition is provided comprising bupropion and a tetracyclic antidepressant formulated into a single dosage formulation. In a further embodiment, the tetracyclic antidepressant is selected from the group consisting of amoxapine and maprotiline.

In yet a further embodiment, a composition is provided comprising lithium and an antipsychotic drug formulated into a single dosage formulation. In another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

In an embodiment, a composition is provided comprising lithium and a monoamine oxidase inhibitor formulated into a single dosage formulation. In a further embodiment, the monoamine oxidase inhibitor is selegiline.

In another embodiment, a composition is provided comprising lithium, bupropion and modafinil in a single dosage formulation.

In yet another embodiment, a composition is provided comprising lithium, bupropion and lamotrigene in a single dosage formulation.

In a further embodiment, a composition is provided comprising bupropion and lamotrigene in a single dose formulation.

In another embodiment, a composition is provided comprising bupropion and an antiseizure drug in a single dosage formulation. In a further embodiment, the antiseizure drug is selected from the group consisting of oxcarbazepine, lamotrigene, topiramate, divalproex sodium, levetiracetam, gabapentin, tiagabine, and zonisamide.

In an embodiment, a method is provided for treating a patient for a mood spectrum disorder comprising administering to the patient an effective amount of bupropion and a tricyclic antidepressant formulated into a single dosage formulation. In a further embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet another embodiment, the method further comprises diagnosing the patient by using a mood disorder screen.

In another embodiment, a method is provided for treating a patient for a mood spectrum disorder comprising administering to the patient an effective amount of bupropion and a tetracyclic antidepressant formulated into a single dosage formulation. In a further embodiment, the tetracyclic antidepressant is selected from the group consisting of amoxapine and maprotiline. In yet another embodiment, the method further comprises diagnosing the patient by using a mood disorder screen.

In an embodiment, a method is provided for treating a patient for a mood spectrum disorder comprising administering to the patient an effective amount of lithium and an antipsychotic drug formulated into a single dosage formulation. In a further embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet another embodiment, the method further comprises diagnosing the patient by using a mood disorder screen.

In yet another embodiment, a method is provided for treating a patient for a mood spectrum disorder comprising administering to the patient an effective amount of lithium and an monoamine oxidase inhibitor formulated into a single dosage formulation. In a further embodiment, the monoamine oxidase inhibitor is selegiline. In yet another embodiment, the method further comprises diagnosing the patient by using a mood disorder screen.

In another embodiment, a method is provided for treating a patient for a mood spectrum disorder comprising administering to the patient an effective amount of lithium, bupropion, and modafinil formulated into a single dosage formulation. In yet another embodiment, the method further comprises diagnosing the patient by using a mood disorder screen.

In an embodiment, a method is provided for treating a patient for a mood spectrum disorder comprising administering to the patient an effective amount of lithium, bupropion, and lamotrigine formulated into a single dosage formulation. In another embodiment, the method further comprises diagnosing the patient by using a mood disorder screen.

In an embodiment, a method is provided for treating a patient for a mood spectrum disorder comprising administering to the patient an effective amount of bupropion and lamotrigine formulated into a single dosage formulation. In another embodiment, the method further comprises diagnosing the patient by using a mood disorder screen.

In another embodiment, a method is provided for treating a patient for a mood spectrum disorder comprising administering to the patient an effective amount of bupropion and an antiseizure drug formulated into a single dosage formulation. In a further embodiment, the antiseizure drug is divalproex sodium. In yet a further embodiment, the method further comprises diagnosing the patient by using a mood disorder screen.

In an embodiment, a method is provided for treating a patient for Bipolar I disorder, depressed type by administering an effective amount of a composition comprising one of the following combinations: (a) bupropion and a tricyclic antidepressant; (b) bupropion and a tetracyclic antidepressant; (c) lithium and an antipsychotic drug; (d) lithium and a monoamine oxidase inhibitor; (e) lithium, bupropion, and modafinil; (f) lithium, bupropion, and lamotrigine; (g) bupropion and lamotrigene; (h) lithium, a selective serotonin reuptake inhibitor and an antiseizure drug; (i) lithium, a selective serotonin reuptake inhibitor and an atypical neuroleptic; (j) lithium, a selective serotonin reuptake inhibitor and an antipsychotic drug; (k) lithium, a selective serotonin reuptake inhibitor and a tricyclic antidepressant; (l) lithium, a selective serotonin reuptake inhibitor and bupropion hydrochloride; (m) lithium, a selective serotonin reuptake inhibitor, a tricyclic antidepressant, and an atypical neuroleptic; (n) lithium, a selective serotonin reuptake inhibitor, a tricyclic antidepressant, an atypical neuroleptic, and an antiseizure drug; (o) lithium and bupropion hydrochloride; (p) lithium and mirtazapine; (q) bupropion hydrochloride and an atypical neuroleptic; and (r) lithium and a tricyclic antidepressant. In a further embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet another embodiment, the tetracyclic antidepressant is selected from the group consisting of amoxapine and maprotiline. In yet a further embodiment, the antipsychotic is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In another embodiment, the monoamine oxidase inhibitor is selected from the group consisting of phenelzine sulfate, tranylcypromine, and selegiline. In a further embodiment, the selective serotonin reuptake inhibitor is selected from the group consisting of citalopram, escitalopram, paroxetine, sertraline, fluoxetine, and duloxetine. In yet another embodiment, antiseizure drug is selected from the group consisting of oxcarbazepine, lamotrigene, topiramate, divalproex sodium, levetiracetam, gabapentin, tiagabine, and zonisamide. In a further embodiment, an atypical neuroleptic is selected from the group consisting of aripiprazole, quetiapine fumarate, risperidone, ziprasidone, and olanzapine.

In another embodiment, a method is provided for treating a patient for Bipolar I disorder, manic type by administering an effective amount of a composition comprising one of the following combinations: (a) lithium, a selected serotonin reuptake inhibitor, and an atypical neuroleptic; (b) lithium, a selected serotonin reuptake inhibitor, and an antipsychotic drug; (c) lithium and an antipsychotic drug; (d) lithium, bupropion, and an atypical neuroleptic; (e) lithium, bupropion and an antiseizure drug; (f) lithium, bupropion, and an antipsychotic drug; (g) lithium, bupropion, an antiseizure drug, and an antipsychotic drug; (h) lithium and a monoamine oxidase inhibitor; (i) lithium, bupropion, and lamotrigine; (j) bupropion and lamotrigine; (k) lithium and bupropion; (l) lithium and mirtazapine; and (m) bupropion and an antiseizure drug. In a further embodiment, the selective serotonin reuptake inhibitor is selected from the group consisting of citalopram, escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine. In yet another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perpheniazine, and molindone hydrochloride. In a further embodiment, the monoamine oxidase inhibitor is selected from the group consisting of phenelzine sulfate, tranylcypromine, and selegiline. In yet a further embodiment, the antiseizure drug is selected from the group consisting of oxcarbazepine, lamotrigene, topiramate, divalproex sodium, levetiracetam, gabapentin, tiagabine, and zonisamide.

In an embodiment, a method is provided for treating a patient for Bipolar I disorder, mixed type, by administering an effective amount of a composition comprising one of the following combinations: (a) bupropion and a tricyclic antidepressant; (b) bupropion and a tetracyclic antidepressant; (c) lithium and an antipsychotic drug; (d) lithium and monoamine oxidase inhibitor; (e) lithium, bupropion, and modafinil; (f) lithium, bupropion, and lamotrigine; (g) bupropion and lamotrigine; (h) lithium and bupropion; (i) lithium and mirtazapine; and (j) bupropion and an antiseizure drug. In another embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet a further embodiment, the tetracyclic antidepressant is selected from the group consisting of amoxapine and maprotiline. In yet another embodiment, the antipsychotic is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In another embodiment, the monoamine oxidase inhibitor is selected from the group consisting of phenelzine sulfate, tranylcypromine, and selegiline. In a further embodiment, antiseizure drug is selected from the group consisting of oxcarbazepine, lamotrigene, topiramate, divalproex sodium, levetiracetam, gabapentin, tiagabine, and zonisamide.

In another embodiment, a method is provided for treating a patient for Bipolar II by administering an effective amount of a composition comprising one of the following combinations: (a) lithium, a selective serotonin reuptake inhibitor, and an atypical neuroleptic; (b) lithium, a selective serotonin reuptake inhibitor, and an antipsychotic drug; (c) lithium, a selective serotonin reuptake inhibitor, and an antiseizure drug; (d) lithium and an antipsychotic drug; (e) lithium and monoamine oxidase inhibitor; and (f) lithium, bupropion, and modafinil. In a further embodiment, the selective serotonin reuptake inhibitor is selected from the group consisting of citalopram, escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine. In another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet another embodiment, the antiseizure drug is selected from the group consisting of oxcarbazepine, lamotrigene, topiramate, divalproex sodium, levetiracetam, gabapentin, tiagabine, and zonisamide. In a further embodiment, the monoamine oxidase inhibitor is selected from the group consisting of phenelzine sulfate, tranylcypromine, and selegiline.

In an embodiment, a method is provided for treating a patient for Borderline Personality Disorder by administering an effective amount of a composition comprising one of the following combinations: (a) lithium, a selective serotonin reuptake inhibitor, and an atypical neuroleptic; (b) lithium, a selective serotonin reuptake inhibitor, and an antipsychotic drug; (c) lithium, a selective serotonin reuptake inhibitor and an antiseizure drug; (d) bupropion and a tricyclic antidepressant; (e) bupropion and a tetracyclic antidepressant; (f) lithium and an antipsychotic drug; (g) lithium and a monoamine oxidase inhibitor; (h) lithium, bupropion, and modafinil; (i) lithium, bupropion, and lamotrigine; 0) bupropion and lamotrigine; (k) lithium and bupropion; and (l) lithium and mirtazapine. In a further embodiment, the selective serotonin reuptake inhibitor is selected from the group consisting of citalopram, escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine. In another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet another embodiment, the antiseizure drug is selected from the group consisting of oxcarbazepine, lamotrigene, topiramate, divalproex sodium, levetiracetam, gabapentin, tiagabine, and zonisamide. In another embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet another embodiment, the tetracyclic antidepressant is selected from the group consisting of amoxapine and maprotiline. In yet a further embodiment, the monoamine oxidase inhibitor is selected from the group consisting of phenelzine sulfate, tranylcypromine, and selegiline.

In another embodiment, a method is provided for treating a patient for Cyclothymia by administering an effective amount of a composition comprising one of the following combinations: (a) lithium, a selective serotonin reuptake inhibitor, and an atypical neuroleptic; (b) lithium, a selective serotonin reuptake inhibitor, and an antipsychotic drug; (c) lithium, a selective serotonin reuptake inhibitor and an antiseizure drug; (d) bupropion and a tricyclic antidepressant; (e) bupropion and a tetracyclic antidepressant; (f) lithium and an antipsychotic drug; (g) lithium and a monoamine oxidase inhibitor; (h) lithium, bupropion, and modafinil; (i) lithium, bupropion, and lamotrigine; (j) bupropion and lamotrigine; (k) lithium and bupropion; and (l) lithium and mirtazapine. In a further embodiment, the selective serotonin reuptake inhibitor is selected from the group consisting of citalopram, escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine. In another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet another embodiment, the antiseizure drug is selected from the group consisting of oxcarbazepine, lamotrigene, topiramate, divalproex sodium, levetiracetam, gabapentin, tiagabine, and zonisamide. In a further embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet a further embodiment, the tetracyclic antidepressant is selected from the group consisting of amoxapine and maprotiline. In another embodiment, the monoamine oxidase inhibitor is selected from the group consisting of phenelzine sulfate, tranylcypromine, and selegiline.

In an embodiment, a method is provided for treating a patient for Depressive Personality Disorder by administering an effective amount of a composition comprising one of the following combinations: (a) lithium, a selective serotonin reuptake inhibitor, and an antiseizure drug; (b) lithium, a selective serotonin reuptake inhibitor, and bupropion (c) bupropion and a tricyclic antidepressant; (d) bupropion and a tetracyclic antidepressant; (e) lithium and an antipsychotic drug; (f) lithium and a monoamine oxidase inhibitor; (g) lithium, bupropion, and modafinil; (h) lithium, bupropion, and lamotrigine; (i) bupropion and lamotrigine; (j) lithium and bupropion; and (k) lithium and mirtazapine. In another embodiment, the selective serotonin reuptake inhibitor is selected from the group consisting of citalopram, escitalopram, paroxetine, sertraline, fluoxetine, and duloxetine. In yet another embodiment, the antiseizure drug is selected from the group consisting of oxcarbazepine, lamotrigene, topiramate, divalproex sodium, levetiracetam, gabapentin, tiagabine, and zonisamide. In a further embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet a further embodiment, the tetracyclic antidepressant is selected from the group consisting of amoxapine and maprotiline. In another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet a further embodiment, the monoamine oxidase inhibitor is selected from the group consisting of phenelzine sulfate, tranylcypromine, and selegiline.

In another embodiment, a method is provided for treating a patient for Dysthymia by administering an effective amount of a composition comprising one of the following combinations: (a) lithium, a selective serotonin reuptake inhibitor, and an antiseizure drug; (b) lithium, a selective serotonin reuptake inhibitor, and an atypical neuroleptic; (c) lithium, a selective serotonin reuptake inhibitor, and an antipsychotic drug (d) lithium and an antipsychotic drug; (e) lithium and bupropion; (f) lithium, an antiseizure drug, and a tricyclic antidepressant; (g) lithium and mirtazapine; (h) lithium and a tricyclic antidepressant; (i) lithium and a monoamine oxidase inhibitor; (j) bupropion and a tricyclic antidepressant; (k) bupropion and a tetracyclic antidepressant; (l) lithium, bupropion, and modafinil; (m) lithium, bupropion, and lamotrigine; and (n) bupropion and lamotrigine. In a further embodiment, the selective serotonin reuptake inhibitor is selected from the group consisting of citalopram, escitalopram, paroxetine, sertraline, fluoxetine, and duloxetine. In yet another embodiment, the antiseizure drug is selected from the group consisting of oxcarbazepine, lamotrigene, topiramate, divalproex sodium, levetiracetam, gabapentin, tiagabine, and zonisamide. In yet a further embodiment, the atypical neuroleptic is selected from the group consisting of aripiprazole, quetiapine fumarate, risperidone, ziprasidone, and olanzapine. In an embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In another embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In a further embodiment, the tetracyclic antidepressant is selected from the group consisting of amoxapine and maprotiline. In yet a further embodiment, the monoamine oxidase inhibitor is selected from the group consisting of phenelzine sulfate, tranylcypromine, and selegiline.

In an embodiment, a method is provided for treating a patient for Eating Disorder by administering an effective amount of a composition comprising one of the following combinations: (a) lithium, a selective serotonin reuptake inhibitor, and an atypical neuroleptic; (b) lithium, a selective serotonin reuptake inhibitor, an atypical neuroleptic, and an antiseizure drug; (c) lithium and an antipsychotic drug; and (d) lithium and a tricyclic antidepressant. In another embodiment, the selective serotonin reuptake inhibitor is selected from the group consisting of citalopram, escitalopram, paroxetine, sertraline, fluoxetine, and duloxetine. In a further embodiment, the atypical neuroleptic is selected from the group consisting of aripiprazole, quetiapine fumarate, risperidone, ziprasidone, and olanzapine. In yet another embodiment, the antiseizure drug is selected from the group consisting of oxcarbazepine, lamotrigene, topiramate, divalproex sodium, levetiracetam, gabapentin, tiagabine, and zonisamide. In yet a further embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In an embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

In an embodiment, a method is provided for treating a patient for General Anxiety Disorder by administering an effective amount of a composition comprising one of the following combinations: (a) lithium, a selective serotonin reuptake inhibitor, and an atypical neuroleptic; and (b) lithium, a selective serotonin reuptake inhibitor, and an antipsychotic drug. In another embodiment, the selective serotonin reuptake inhibitor is selected from the group consisting of citalopram, escitalopram, paroxetine, sertraline, fluoxetine, and duloxetine. In a further embodiment, the atypical neuroleptic is selected from the group consisting of aripiprazole, quetiapine fumarate, risperidone, ziprasidone, and olanzapine. In yet a further embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

In an embodiment, a method is provided for treating a patient for Major Depressive Disorder by administering an effective amount of a composition comprising one of the following combinations: (a) lithium and a tricyclic antidepressant; (b) lithium and a monoamine oxidase inhibitor; (c) lithium, a tricyclic antidepressant and an antiseizure drug; (d) lithium and bupropion; (e) lithium and mirtazapine; (f) lithium, a selective serotonin reuptake inhibitor, and a tricyclic antidepressant; (g) lithium, a selective serotonin reuptake inhibitor, a tricyclic antidepressant, and an atypical neuroleptic; (h) lithium, a selective serotonin reuptake inhibitor, a tricyclic antidepressant, and an antiseizure drug; (i) lithium, a selective serotonin reuptake inhibitor, a tricyclic antidepressant, an atypical neuroleptic and an antiseizure drug; and (j) lithium, bupropion and modafinil. In another embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In a further embodiment, the monoamine oxidase inhibitor is selected from the group consisting of phenelzine sulfate, tranylcypromine, and selegiline. In yet another embodiment, the antiseizure drug is selected from the group consisting of oxcarbazepine, lamotrigene, topiramate, divalproex sodium, levetiracetam, gabapentin, tiagabine, and zonisamide. In yet a further embodiment, the selective serotonin reuptake inhibitor is selected from the group consisting of citalopram, escitalopram, paroxetine, sertraline, fluoxetine, and duloxetine. In an embodiment, the atypical neuroleptic is selected from the group consisting of aripiprazole, quetiapine fumarate, risperidone, ziprasidone, and olanzapine.

In another embodiment, a method is provided for treating a patient for Panic Disorder by administering an effective amount of a composition comprising lithium, a selective serotonin reuptake inhibitor, and an atypical neuroleptic. In a further embodiment, the selective serotonin reuptake inhibitor is selected from the group consisting of citalopram, escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine.

In another embodiment, a method is provided for treating a patient for Posttraumatic Stress Disorder by administering an effective amount of a composition comprising lithium, a selective serotonin reuptake inhibitor, and an atypical neuroleptic. In a further embodiment, the selective serotonin reuptake inhibitor is selected from the group consisting of citalopram, escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine.

In an embodiment, a method is provided for treating a patient for Pre-Menstrual Dysphoric Disorder by administering an effective amount of a composition comprising one of the following combinations: (a) lithium, a selective serotonin reuptake inhibitor, and an antiseizure drug; (b) lithium, a selective serotonin reuptake inhibitor, and an atypical neuroleptic; and (c) lithium and bupropion. In another embodiment, the selective serotonin reuptake inhibitor is selected from the group consisting of citalopram, escitalopram, paroxetine, sertraline, fluoxetine, and duloxetine. In a further embodiment, wherein the antiseizure drug is selected from the group consisting of oxcarbazepine, lamotrigene, topiramate, divalproex sodium, levetiracetam, gabapentin, tiagabine, and zonisamide. In yet another embodiment, the atypical neuroleptic is selected from the group consisting of aripiprazole, quetiapine fumarate, risperidone, ziprasidone, and olanzapine.

In another embodiment, a method is provided for treating a patient for Schizoaffective Disorder, depressed type, by administering an effective amount of a composition comprising one of the following combinations: (a) lithium and an antipsychotic drug; (b) lithium, a selective serotonin reuptake inhibitor, and an atypical neuroleptic; (c) lithium, a selective serotonin reuptake inhibitor, and an antipsychotic drug; (d) lithium, a selective serotonin reuptake inhibitor, an antipsychotic drug, and an antiseizure drug; (e) lithium, a selective serotonin reuptake inhibitor, an atypical neuroleptic, and an antiseizure drug; (f) lithium, a selective serotonin reuptake inhibitor, and bupropion; (g) lithium, a selective serotonin reuptake inhibitor, bupropion and an atypical neuroleptic; and (h) lithium, a selective serotonin reuptake inhibitor, bupropion and an antipsychotic drug. In an embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In another embodiment, the atypical neuroleptic is selected from the group consisting of aripiprazole, quetiapine fumarate, risperidone, ziprasidone, and olanzapine. In yet another embodiment, wherein the selective serotonin reuptake inhibitor is selected from the group consisting of citalopram, escitalopram, paroxetine, sertraline, fluoxetine, and duloxetine. In yet a further embodiment, the antiseizure drug is selected from the group consisting of oxcarbazepine, lamotrigene, topiramate, divalproex sodium, levetiracetam, gabapentin, tiagabine, and zonisamide.

In another embodiment, a method is provided for treating a patient for Schizoaffective Disorder, manic type, by administering an effective amount of a composition comprising one of the following combinations: (a) lithium and an antipsychotic drug; (b) lithium, an atypical neuroleptic, and an antiseizure drug; (c) lithium, an antipsychotic drug and an antiseizure drug; (d) lithium, bupropion and an atypical neuroleptic; (e) lithium bupropion and an antipsychotic drug; (f) lithium, bupropion, an atypical neuroleptic and an antiseizure drug; (g) lithium, bupropion, an antipsychotic drug and an antiseizure drug; (h) lithium, a selective serotonin reuptake inhibitor, and an atypical neuroleptic; (i) lithium, a selective serotonin reuptake inhibitor, and an antipsychotic drug; (j) lithium, a selective serotonin reuptake inhibitor, and an antiseizure drug; (k) lithium, a selective serotonin reuptake inhibitor, an atypical neuroleptic and an antipsychotic drug; and (l) bupropion and an antiseizure drug. In a further embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet a further embodiment, the atypical neuroleptic is selected from the group consisting of aripiprazole, quetiapine fumarate, risperidone, ziprasidone, and olanzapine. In another embodiment, the antiseizure drug is selected from the group consisting of oxcarbazepine, lamotrigene, topiramate, divalproex sodium, levetiracetam, gabapentin, tiagabine, and zonisamide. In yet another embodiment, the selective serotonin reuptake inhibitor is selected from the group consisting of citalopram, escitalopram, paroxetine, sertraline, fluoxetine, and duloxetine. In a further embodiment, the antiseizure drug is selected from the group consisting of oxcarbazepine, lamotrigene, topiramate, divalproex sodium, levetiracetam, gabapentin, tiagabine, and zonisamide.

In an embodiment, a method is provided for treating a patient for Schizoaffective Disorder, mixed type, by administering an effective amount of a composition comprising one of the following combinations: (a) bupropion and an antiseizure drug; (b) lithium, and an antipsychotic drug; (c) lithium, an atypical neuroleptic, and an antiseizure drug; (d) lithium, an antipsychotic drug, and an antiseizure drug; (e) lithium, bupropion, and an atypical neuroleptic; (f) lithium, bupropion, and an antipsychotic drug; (g) lithium, bupropion, an atypical neuroleptic, and an antiseizure drug; (h) lithium, bupropion, an antipsychotic drug, and an antiseizure drug; (i) lithium, a selective serotonin reuptake inhibitor, and an atypical neuroleptic; (j) lithium, a selective serotonin reuptake inhibitor, and an antipsychotic drug; (k) lithium, a selective serotonin reuptake inhibitor, an atypical neuroleptic, and an antiseizure drug; and (l) lithium, a selective serotonin reuptake inhibitor, an antipsychotic drug, and antiseizure drug. In another embodiment, the antiseizure drug is selected from the group consisting of oxcarbazepine, lamotrigene, topiramate, divalproex sodium, levetiracetam, gabapentin, tiagabine, and zonisamide. In yet another embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In a further embodiment, the atypical neuroleptic is selected from the group consisting of aripiprazole, quetiapine fumarate, risperidone, ziprasidone, and olanzapine. In yet a further embodiment, the selective serotonin reuptake inhibitor is selected from the group consisting of citalopram, escitalopram, paroxetine, sertraline, fluoxetine, and duloxetine.

In another embodiment, a method is provided for treating a patient for Seasonal Affective Disorder by administering an effective amount of a composition comprising one of the following combinations: (a) lithium, a selective serotonin reuptake inhibitor, and an atypical neuroleptic; (b) lithium, a selective serotonin reuptake inhibitor, and an antiseizure drug; (c) lithium and bupropion; (d) lithium, bupropion and an atypical neuroleptic; and (e) lithium and an tricyclic antidepressant. In a further embodiment, the selective serotonin reuptake inhibitor is selected from the group consisting of citalopram. escitalopram, paroxetine, sertraline, fluoxetine, and duloxetine. In yet a further embodiment, the atypical neuroleptic is selected from the group consisting of aripiprazole, quetiapine fumarate, risperidone, ziprasidone, and olanzapine. In an embodiment, the antiseizure drug is selected from the group consisting of oxcarbazepine, lamotrigene, topiramate, divalproex sodium, levetiracetam, gabapentin, tiagabine, and zonisamide. In another embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In a further embodiment, the selective serotonin reuptake inhibitor is selected from the group consisting of citalopram, escitalopram, paroxetine, sertraline, fluoxetine, and duloxetine.

In an embodiment, a method is provided for treating a patient for Social Anxiety Disorder by administering an effective amount of a composition comprising one of the following combinations: (a) lithium, a selective serotonin reuptake inhibitor, and an atypical neuroleptic; and (b) lithium, a selective serotonin reuptake inhibitor, and an antipsychotic drug. In an another embodiment, the selective serotonin reuptake inhibitor is selected from the group consisting of citalopram, escitalopram, paroxetine, sertraline, fluoxetine, and duloxetine. In a further embodiment, the atypical neuroleptic is selected from the group consisting of aripiprazole, quetiapine fumarate, risperidone, ziprasidone, and olanzapine. In yet a further embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

In another embodiment, a method is provided for treating a patient for Substance Use Disorder by administering an effective amount of a composition comprising one of the following combinations: (a) lithium and bupropion; (b) lithium, bupropion, and an atypical neuroleptic; (c) lithium, bupropion and an antipsychotic drug; (d) lithium, bupropion, and an antiseizure drug; (e) lithium, a selective serotonin reuptake inhibitor, and an antipsychotic drug; (f) lithium, a selective serotonin reuptake inhibitor, and an antiseizure drug; (g) lithium, a selective serotonin reuptake inhibitor, and an atypical neuroleptic; (h) lithium, a selective serotonin reuptake inhibitor, an antiseizure drug, and an atypical neuroleptic; (i) lithium, a selective serotonin reuptake inhibitor, and a tricyclic antidepressant; (j) lithium and a monoamine oxidase inhibitor; (k) bupropion and a tricyclic antidepressant; (l) bupropion and a tetracyclic antidepressant; (m) lithium and an antipsychotic drug; (n) lithium, bupropion, and modafinil,; (o) lithium, bupropion, and lamotrigine; (p) bupropion and lamotrigine; and (q) lithium and mirtazapine. In a further embodiment, the atypical neuroleptic is selected from the group consisting of aripiprazole, quetiapine fumarate, risperidone, ziprasidone, and olanzapine. In yet a further embodiment, the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride. In yet another embodiment, the antiseizure drug is selected from the group consisting of oxcarbazepine, lamotrigene, topiramate, divalproex sodium, levetiracetam, gabapentin, tiagabine, and zonisamide. In an embodiment, the selective serotonin reuptake inhibitor is selected from the group consisting of citalopram, escitalopram, paroxetine, sertraline, fluoxetine, and duloxetine. In another embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In yet another embodiment, the tetracyclic antidepressant is selected from the group consisting of amoxapine and maprotiline. In a further embodiment, the monoamine oxidase inhibitor is selected from the group consisting of phenelzine sulfate, tranylcypromine, and selegiline.

In an embodiment, a method is provided for treating a patient for Treatment Resistant Depression by administering an effective amount of a composition comprising one of the following combinations: (a) lithium, a selective serotonin reuptake inhibitor, and an atypical neuroleptic; (b) lithium, a selective serotonin reuptake inhibitor, and an antiseizure drug; (c) lithium, a selective serotonin reuptake inhibitor, and an tricyclic antidepressant; (d) lithium and bupropion; (e) lithium, bupropion and an atypical neuroleptic; (f) lithium, bupropion, and an antiseizure drug; (g) lithium and a monoamine oxidase inhibitor; (h) lithium and a tricyclic antidepressant; (i) lithium, a tricyclic antidepressant, and an atypical neuroleptic; (j) lithium, a tricyclic antidepressant, an atypical neuroleptic, and an antiseizure drug; (k) lithium, a selective serotonin reuptake inhibitor and venlafaxine; (l) lithium, a selective serotonin reuptake inhibitor, and bupropion; (m) lithium, a selective serotonin reuptake inhibitor, venlafaxine and an antiseizure drug; (n) lithium, a selective serotonin reuptake inhibitor, bupropion and an antiseizure drug; (o) bupropion and a tricyclic antidepressant; (p) bupropion and a tetracyclic antidepressant; (q) lithium and antipsychotic drug; (r) lithium, bupropion, and modafinil; (s) lithium, bupropion, and lamotrigine; (t) bupropion and lamotrigine; and (u) lithium and mirtazapine. In another embodiment, the selective serotonin reuptake inhibitor is selected from the group consisting of citalopram, escitalopram, paroxetine, sertraline, fluoxetine, and duloxetine. In a further embodiment, the atypical neuroleptic is selected from the group consisting of aripiprazole, quetiapine fumarate, risperidone, ziprasidone, and olanzapine. In yet a further embodiment, the antiseizure drug is selected from the group consisting of oxcarbazepine, lamotrigene, topiramate, divalproex sodium, levetiracetam, gabapentin, tiagabine, and zonisamide. In yet a further embodiment, the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline. In another embodiment, the monoamine oxidase inhibitor is selected from the group consisting of phenelzine sulfate, tranylcypromine, and selegiline. In an embodiment, the tetracyclic antidepressant is selected from the group consisting of amoxapine and maprotiline.

### DETAILED DESCRIPTION OF THE INVENTION

### I. Introduction

There are a great many possible uses of the invention to treat highly refractory, often undiagnosed, serious psychiatric illnesses. The invention's compositions and methods would enable such treatments to be undertaken by PCP's utilizing the invention's mental health diagnostic and cost-containing drug compositions. The methods and compositions described herein can be initiated immediately on the very day a PCP makes the diagnosis, thus insuring that mood disordered patients are appropriately diagnosed and that treatment can be initiated immediately. This prevents the possibility of patients failing to follow through with the PCPs' referrals to psychiatrists.

Additionally, in particular embodiments, the invention would be an excellent drug in the very difficult to treat Borderline Personality Disorder. This disorder often includes cyclothymia, rages, impulsivity, self-mutilative behavior, chronic separation anxiety and chronic low self-esteem. All these symptoms would be affected in a positive manner by the synergistic drug combinations contained in the invention.

Other embodiments allow the majority of a heretofore undiagnosed or under-diagnosed, misdiagnosed, under-treated or untreated mentally ill population to be properly identified as suffering from mood disorder. Even if a patient refuses his PCP's recommendation for treatment, that patient's medical record will always contain the Mood Disorder Screening Test results which can be critical in determining appropriate future treatment. The invention focuses on the use of PCPs rather than specialists by giving PCPs a drug technology that is simple enough to initiate the treatment of mood disorder.

Thus, early diagnosis and preventative treatment will impact the treatment mood disorder while the cost of such treatment will be dramatically reduced.

### II. Mood Spectrum Disorders

The DSM-IV describes mood disorder as a set of discrete mood disorders treated with discrete treatment regimens. See DSM-IV-TR, 4^{th} ed., p. 345-428 (2000). Mood disorder, however, is a continuum. At any one time, a given patient may present with what appears to be Major Depressive Disorder (“MDD”), yet at another time, that patient may present with dysthymia, followed by a third presentation that might be pre-menstrual dysphoric disorder (“PMDD”), bipolar II disorder, or seasonal affective disorder. Thus, unlike the DSM-IV, the invention teaches that mood disorder is a continuum. This continuity explains why complex co-morbidities are very common in mood disorders. Examples of such co-morbidities include depression, anxiety, major depressive disorder (“MDD”), obsessive compulsive disorder (“OCD”), general anxiety disorder (“GAD”), panic disorder (“PD”), PMDD, substance use disorder (“SUD”), social anxiety disorder (“SAD”), treatment resistant depression, post traumatic stress disorder (“PTSD”), bipolar disorder, borderline personality disorder (“BPD”), depressive personality disorder (“DPD”), and dysthymia. Thus, particular embodiments treat mood disorder as the disregulation of any affect, including, sadness, anger, joy, anxiety, fear, guilt, and shame.

Thus, the same patient may present at different times with varying admixtures of mood disregulation, and may then be successfully treated with the compositions described herein. The interconnection of affective disorders can clearly be seen in the relationship between dysthymia and MDD. More than 60% of dysthymics experience MDD while 40% of patients with MDD suffer from “double” depressions that include dysthymia.

Although many patients appear to need only antidepressant treatment, many of these patients will do better if treated with the compositions described herein wherein a combination of lithium and antidepressant is provided. Further, patients who present with one particular manifestation of mood disorder may best be treated for long-term prevention of the comorbid forms of mood disorder. MDD, recurrent depression, may be best viewed as a disorder of mood regulation that may be better treated with a combination of mood stabilizer and antidepressant. The use of the methods will help the 50 percent of MDD patients who currently are under-treated, whether or not these patients are actually suffering from Bipolar I or II Disorder or suffer from “double depression.” “Bipolar I disorder is expressed as a dimensional illness featuring the full range [spectrum] of affective symptom severity and polarity.” Judd et al., Arc. Gen. Psychiatry, 59:530 (2002). This same study cites that 60% of patients with MDD also suffered from subsyndromal depression during their “remission.” The DSM-IV conceptualization of discrete mood episodes is not the natural history of psychiatric mood disorders. Judd, el al., Arc. Gen. Psychiatry, 55:694-700 (1998). Thus, mood disorders that include bipolar I, depressed, manic, and mixed types; MDD, antidepressant-induced mania (“AIM”), hypomania, cyclothymia, SUD's, impulse control disorder, DPD, subsyndromal dysthymia and bipolar II, all appear as part of mood spectrum disorder illness that requires lifetime maintenance. DSM-IV criteria are outdated and further complicate the diagnosis and treatment of mood disorders. The invention's methods and compositions will enable PCPs to move forward in the treatment of tens of millions of undiagnosed or misdiagnosed patients who suffer from mood disorder.

Further, particular embodiments underscore the primacy of relapse prevention. Whereby, the need for more frequent utilization of a mood stabilizing drug, beyond just the treatment of a discrete mood disorder, such as bipolar illness, becomes critical. Use of the invention's methods would be effective in preventing relapse, while achieving recovery and remission. More frequent use of lithium, through the use of the compositions described herein, will provide patients with both a “ceiling” and a “floor” to contain the intensity of all of their moods, regardless of the level of current life stressors. Therefore, in a particular embodiment, for example, wherein lithium augmentation of citalopram is used in the treatment of MDD, dysthymia and other mood disorders, patients will have better acute and chronic outcomes, with fewer and less severe recurrences along with significant decrease in suicide rates.

### A. Dysthymia

Dysthymia is a disorder with similar, but longer-lasting and milder symptoms than clinical depression. By the standard psychiatric definition, this disorder lasts for at least two years, but is less disabling than major depression; for example, victims are usually able to go on working and do not need to be hospitalized. The incidence of dysthymia in the general population is about 5%. Dysthymia occurs twice as often in women as it does in men and it is also more common among the poor and the unmarried. The symptoms usually appear in adolescence or young adulthood, but in some cases symptoms do not emerge until middle age. Nevertheless, the large incidence, unrelenting chronicity, frequent undiagnosis and high lifetime suicide rates, makes the treatment of dysthymia a critical mental health priority.

Dysthymia is co-morbid in the so-called “double depression” that complicates forty percent of all patients diagnosed with MDD. Dysthymia is not “minor.” Studies have shown dysthymia to be more damaging to both intrapersonal and interpersonal patient functioning than MDD. Greater than 60% of patients found in psychiatric clinics suffer from dysthymia. The lifetime incidence of completed suicide in dysthymics exceeds that in either MDD or bipolar illness and is second only to the rate of suicide in psychotic depression. Dysthymia has proven to be an extremely difficult illness to treat. A number of studies, however, point to the effectiveness of SSRIs in the treatment of dysthymia. Nevertheless, the 50% success rate of SSRIs in the treatment of dysthymia could be enhanced with lithium augmentation. Kaplan and Sadock, “Synopsis of Psychiatry,” 7^{th} Ed., Williams and Wilkins p. 997.

In a one-year follow-up study of dysthymia quoted in the current edition of Sadock and Kaplan's Synopsis of Psychiatry (8^{th} Ed., p. 577 (1998)), only 15% of dysthymic patients were in remission. This data strongly suggests the need for more effective treatment agents like the invention's combination drugs. The data also suggests the need for long-term maintenance treatment. Anxiety disorders, as well as SUD and axis II diagnoses, including borderline and narcissistic personality disorders, often are co-morbid with dysthymia and should respond well to particular compositions described herein.

Particular embodiments allow diagnosis and scoring of a simple questionnaire that will permit PCPs to successfully identify and treat most cases of dysthymia. A particular embodiment of the Dysthymia Screen appears in Example 2. The patients would then be treated with the compositions described herein including, but not limited to: Li/SSRI; Li/SSRI/Antiseizure; Li/SSRI/Atypical Neuroleptic; Li/SSRI/Antipsychotic; Li/SSRI/Antiseizure; Li/Antiseizure; Li/Atypical Neuroleptic; Li/Antipsychotic; Li/Bupropion; Li/Antiseizure/Tricyclic Antidepressant; Li/Mirtazapine; Li/Tricyclic Antidepressant; Li/MAOI; Bupropion/Tricyclic Antidepressant; Bupropion/Tetracyclic Antidepressant; Li/Bupropion/Modafinil; Li/Bupropion/Lamotrigine; and Bupropion/Lamotrigine.

### B. Bipolar Disorder

1. Bipolar I Disorder

Bipolar I disorder, also known as manic-depressive illness, is a brain disorder that causes unusual shifts in a person's mood, energy and ability to function. About 1% of the population age 18 and older has bipolar disorder. Regier et al., Archives of General Psychiatry, 50:85-94 (1993). Bipolar disorder typically develops in late adolescence or early adulthood, but symptoms may appear in childhood or later in life. Bipolar disorder is a long-term illness that must be carefully managed throughout a person's life.

Bipolar disorder is marked by dramatic mood swings ranging from being overly “high,” euphoric and/or irritable to sad and hopeless, and then back again, often with periods of normal mood in between. Severe changes in energy and behavior go along with the changes in mood. The periods of high and lows are called episodes of mania and depression.

Manic episodes can include increased energy, activity and restlessness; feeling “high” or euphoric; being extremely irritable; racing thoughts and talking very fast, jumping from one idea to another; distractibility and inability to concentrate; needing little sleep; unrealistic beliefs in one's abilities and powers; poor judgment; spending sprees; a lasting period of behavior that is different from usual; increased sex drive; abuse of drugs, particularly cocaine, alcohol and sleeping medications; provocative, intrusive or aggressive behavior; poor judgment and denial that anything is wrong.

Depressive episodes include lasting sad, anxious or empty mood; feelings of hopelessness or pessimism; feelings of guilt, worthlessness, or helplessness; loss of interest or pleasure in activities once enjoyed; decreased energy; difficulty concentrating, remembering or being able to make decisions; restlessness or irritability; sleeping too much or inability to sleep; change in appetite and/or unintended weight loss or gain; chronic pain or persistent bodily symptoms not caused by physical illness or injury; and thoughts of death or suicide.

Bipolar I disorders are divided between depressed, manic, and mixed types with approximate incidences of 30%, 60% and 10%, respectively. Bipolar I disorder is the classic form of the illness, which involves recurrent episodes of mania and depression. DSM-IV-TR, 4^{th} ed., p. 382 (2000). Mixed type is characterized by a patient who meets the criteria of bipolar I, depressed type, but also has significant periods of major depressive disorder, or, meets the criteria of bipolar I, manic type, but also has significant depressive mood episodes. Without treatment, the natural course of bipolar disorder tends to worsen. DSM-IV-TR, 4^{th} ed., p. 427 (2000). Over time a person may suffer more frequent and more severe manic and depressive episodes than those experienced when the illness first appeared. Goodwin and Jamison, Manic-Depressive Illness, New York, Oxford University Press, 1990.

When four or more manic/depressive episodes occur within a twelve month period, the individual is said to have rapid-cycling bipolar disorder. Rapid cycling tends to develop later in the course of the illness and is more common among women than among men.

The acute phase of bipolar I is characterized by a mood episode of significant depression or mania. The maintenance phase follows response to treatment and is characterized by a 50% diminution of symptoms; remission occurs when the patient returns to baseline or has a 100% lessening of symptoms.

Particular embodiments allow diagnosis, scoring of a simple questionnaire that will permit PCPs to successfully identify, and treating most cases of bipolar disorder. A particular embodiment of the Bipolar Screen appears in Example 1.

The patients with Bipolar I Disorder, depressed type, in both acute and maintenance treatment phases, would then be treated with the compositions described herein including, but not limited to: Li/SSRI; Li/SSRI/Antiseizure; Li/SSRI/Atypical Neuroleptic; Li/SSRI/Antipsychotic; Li/SSRI/Tricyclic Antidepressant; Li/SSRI/Bupropion; Li/S SRI/Tricyclic Antidepressant/Atypical Neuroleptic; Li/SSRI/Tricyclic Antidepressant/Atypical Neuroleptic/Antiseizure; Li/Bupropion; Li/Mirtazapine; Bupropion/Atypical Neuroleptic; Li/Antiseizure; Li/Atypical Neuroleptic, and Li/Tricyclic Antidepressant; Bupropion/Tricyclic Antidepressant; Bupropion/Tetracyclic Antidepressant; Li/Antipsychotic; Li/MAOI; Li/Bupropion/Modafinil; Li/Bupropion/Lamotrigine; Bupropion/Lamotrigine; and Li/Mirtazapine.

The patients with Bipolar I Disorder, manic type, in both acute and maintenance treatment phases, would then be treated with the compositions described herein including, but not limited to: Li/SSRI; Li/SSRI/Atypical Neuroleptic; Li/Antiseizure; Li/SSRI/Antipsychotic; Li/Atypical Neuroleptic; Li/Antipsychotic; Li/Bupropion/Atypical Neuroleptic; Li/Bupropion/Antiseizure; Li/Bupropion/Antipsychotic; and Li/Bupropion/Antiseizure/Antipsychotic; Li/MAOI; Li/Bupropion/Lamotrigine; Bupropion/Lamotrigine; Li/Bupropion; Li/Mirtazapine; and Bupropion/Antiseizure drug.

The patients with Bipolar I Disorder, mixed type, in both acute and maintenance treatment phases, would then be treated with the compositions described herein including, but not limited to: Bupropion/Tricyclic Antidepressant; Bupropion/Tetracyclic Antidepressant; Li/Antipsychotic; Li/MAOI; Li/Bupropion/Modafinil; Li/Bupropion/Lamotrigine; Bupropion/Lamotrigine; Li/Bupropion; Li/Mirtazapine; and Bupropion/Antiseizure drug.

2. Bipolar II Disorder

Bipolar II is characterized by the presence or history of one or more major depressive episodes and the presence or history of at least one hypomanic episode that alternates with recurrent depressive episodes. The presence of manic or mixed episode precludes the diagnosis of Bipolar TI Disorder. The mood symptoms from the major depressive episodes or the hypomanic episode cannot be better accounted for by schizoaffective disorder and are not superimposed on schizophrenia, schizophreniform disorder, delusional disorder, or psychotic disorder not otherwise specified. The symptoms typically cause clinically significant distress or impairment in social, occupational, or other important areas of functioning.

The patients with Bipolar II Disorder, would then be treated with the compositions described herein including, but not limited to: Li/SSRI; Li/SSRI/Atypical Neuroleptic; Li/SSRI/Antipsychotic; Li/SSRI/Antiseizure; Li/Atypical Neuroleptic; Li/Antipsychotic; Li/Antiseizure; Li/MAOI; and Li/Bupropion/Modafinil.

### C. Treatment Resistant Depression

According to Sadock and Kaplan, treatment-resistant depression is evident when an “antidepressant has been used for four weeks at maximal dosage without therapeutic effect and if plasma levels are adequate.” Kaplan and Sadock's, Synopsis of Psychiatry 7th Ed, Williams and Wilkins, p. 997 (1994). Up to 46% patients with depression do not respond to their initial antidepressant treatment or only responded partially. Residual symptoms are also associated with a greater risk of relapse. Treatment resistant depression is therefore common and a difficult clinical challenge. Rapaport, “The Patient with Treatment Resistant Depression” Teleconference, Apr. 17, 2004, University of Florida.

Treatment resistant depression also includes many cases of “chronic depression.” MDD has been traditionally thought of as an episodic, remitting illness. MDD, however, often has a chronic course with protracted episodes or incomplete remission between episodes. At any given time, at least 3% of the U.S. population suffers from chronic depression. Keller et al., New England Journal of Medicine, 342:1462-1463 (2000). Chronic forms of MDD are associated with more marked impairments in psychosocial functioning and work performance, increased health care utilization, and more frequent suicide attempts and hospitalizations than acute depression. Keller et al., New England Journal of Medicine, 342:1462-1463 (2000). Chronically depressed individuals frequently have onset early in life and are often life-long sufferers. Chronic forms of depression account for an inordinate proportion of the enormous burden of illness associated with depression. Keller et al., New England Journal of Medicine, 342:1462-1463 (2000). The acute phase of treatment resistant depression is characterized by a mood episode of significant depression. The maintenance phase of treatment resistant depression follows response to treatment and is characterized by a 50% diminution of symptoms, while remission occurs when the patient returns to baseline or has a 100% lessening of symptoms.

According to particular embodiments, supplementation of antidepressants with lithium (900-1200 mg/day, serum level between 0.6 and 1.2 mEq per L) for at least 7 to 14 days can be used to treat treatment-resistant depression. This supplementation converts a significant number of treatment-resistant depressive patients into responders. The mechanism of action is not known, and it is not necessary for use of the invention, but lithium may potentiate the serotonergic neuronal system. Kaplan and Sadock's, Synopsis of Psychiatry 7th Ed, Williams and Wilkins, p. 997 (1994). Baumann reports that up to 30% of all depressed patients fail to respond to any given SSRI antidepressant. While lithium has been shown to reduce suicide in depressed patients, it seems to be a poor antidepressant when given alone. Baumann, “Pharmacopsychiatry”, 2001, 34:119-127. Kallner, et al., Pharmacopsychiat. 33:8-13 (2000). A double blind placebo controlled study of citalopram with and without lithium in treatment resistant depression noted that 45 of 69 patients responded to lithium augmentation often within just a few days of the initiation of lithium treatment. Baumann, et al., J. Clin. Psychopharm. 16 (4):307-414 (2001).

Particular embodiments described herein allows diagnosis and scoring of a simple questionnaire that will permit PCPs to successfully identify and treat most cases of treatment resistant depression. The patients in both acute and maintenance treatment phases would then be treated with the compositions described herein including, but not limited to: Li/SSRI; Li/SSRI/Atypical Neuroleptic; Li/SSRI/Antiseizure; Li/SSRI/Tricyclic Antidepressant; Li/Bupropion; Li/Bupropion/Antipsychotic; Li/Bupropion/Atypical Neuroleptic; Li/Bupropion/Antiseizure; Li/MAOI; Li/Tricyclic Antidepressant; Li/Tricyclic Antidepressant/Atypical Neuroleptic; Li/Tricyclic Antidepressant/Atypical Neuroleptic/Antiseizure; Li/S SRI/Venlafaxine; Li/SSRI/Bupropion; Li/SSRI/Venlafaxine/Antiseizure; Li/SSRI/Bupropion/Antiseizure; Bupropion/Tricyclic Antidepressant; Bupropion/Tetracyclic Antidepressant; Li/Antipsychotic; Li/Bupropion/Modafinil; Li/Bupropion/Lamotrigine; Bupropion/Lamotrigine; and Li/Mirtazapine.

### D. Major Depressive Disorder

Major Depressive Disorder is characterized by one or more major depressive episodes without a history of manic, mixed or hypomanic episodes. If manic, mixed or hypomanic episodes develop in the course of MDD, then the diagnosis is changed to bipolar disorder. See DSM-IV-TR, 4^{th} ed., p. 369-376 (2000).

MDD is associated with a high mortality, wherein up to 15% of sufferers die by suicide. MDD may be preceded by dysthymia. It is estimated that each year approximately 10% of individuals with dysthymia will go on to have a first major depressive episode. Other disorders are often comorbid with MDD such as, substance related disorders, PD, OCD, anorexia nervosa, bulimia nervosa and BPD. See DSM-IV-TR, 4^{th} ed., p.369-376 (2000).

MDD is the most common psychiatric disorder in the U.S. with a lifetime prevalence of 16.2%, which corresponds to approximately 32.6-35.1 million U.S. adults. Kessler, et al., Arch Gen. Psychiatry 51:8-19 (1994). MDD may begin at any age, with an average onset in the mid-20's. At least 60% of individuals with MDD, single episode, can be expected to have a second episode. Individuals who have had two episodes have a 70% chance of having a third, and those with a third have a 90% chance of having a fourth. About 5-10% of those with MDD subsequently develop bipolar I disorder. See DSM-IV-TR, 4^{th} ed., p.369-376 (2000).

The diagnostic criteria for 296.2×MDD, Single Episode according to DSM-IV is the following:
A. Presence of a single major depressive episode.

B. The major depressive episode is not better accounted for by Schizoaffective Disorder and is not superimposed on Schizophrenia, Schizophreniform disorder, Delusional Disorder, or Psychotic Disorder Not Otherwise Specified.
C. There has never been a Manic Episode, a Mixed Episode or a Hypomanic Episode. Note: This exclusion does not apply if all of the manic-like, mixed-like, or hypomanic-like episodes are substance or treatment induced or are due to the direct physiological effects of a general medical condition.

If the full criteria are met for a Major Depressive Episode, specify if its current clinical status and/or features: (1) mild, moderate, severe without psychotic features/serve with psychotic features; (2) chronic; (3) with catatonic features; (4) with melancholic features; (5) with atypical features; or (6) with postpartum onset.

If the full criteria are not met for Major Depressive Episode, specify the current clinical status of the Major Depressive Disorder or features of the most recent episode: (1) in partial remission, in full remission; (2) chronic; (3) with catatonic features; (4) with melancholic features; (5) with atypical features; or (6) with postpartum onset.

The diagnostic criteria for 296.3×Major Depressive Disorder, Recurrent according to DSM-IV is as follows:
A. Presence of two or more Major Depressive Episodes. Note: To be considered separate episodes, there must be an interval of at least 2 consecutive months in which criteria are not met for a Major Depressive Episode.
B. The Major Depressive Episodes are not better accounted for by Schizoaffective Disorder and are not superimposed on Schizophrenia, Schizophreniform Disorder, Delusional Disorder, or Psychotic Disorder Not Otherwise Specified.
C. There has never been a Manic Episode, a Mixed Episode, or Hypomanic Episode. Note: This exclusion does not apply if all of the manic-like, mixed-like, or hypomanic-like episodes are substance or treatment induced or are due to the direct physiological effects of a general medical condition.

If the full criteria are met for a Major Depressive Episode, specify if its current clinical status and/or features: (1) mild, moderate, severe without psychotic features/serve with psychotic features; (2) chronic; (3) with catatonic features; (4) with melancholic features; (5) with atypical features; or (6) with postpartum onset.

If the full criteria are not currently met for Major Depressive Episode, specify the current clinical status of the Major Depressive Disorder or features of the most recent episode: (1) in partial remission, in full remission; (2) chronic; (3) with catatonic features; (4) with melancholic features; (5) with atypical features; or (6) with postpartum onset.

Specify: (1) longitudinal course specifiers (with and without interepisode recovery); (2) with seasonal pattern.

Particular embodiments allow diagnosis and scoring of a simple questionnaire that will permit PCPs to successfully identify and treat most cases of MDD. The patients would then be treated with the compositions described herein including, but not limited to: Li/SSRI; Li/Tricyclic Antidepressant; Li/MAOI; Li/Antiseizure; Li/Tricyclic Antidepressant/Antiseizure; Li/Bupropion; Li/Mirtazapine; Li/S SRI/Tricyclic Antidepressant; Li/SSRI/Tricyclic Antidepressant/Atypical Neuroleptic; Li/S SRI/Tricyclic Antidepressant/Antiseizure; Li/SSRI/Tricyclic Antidepressant/Atypical Neuroleptic/Antiseizure; and Li/Bupropion/Modafinil.

### E. General Anxiety Disorder

According to the DSM-IV, Generalized Anxiety Disorder is characterized by excessive anxiety and worry occurring more days than not for a period of at least 6 months about a number of events or activities. In addition to difficulty in controlling the worry, there are three additional symptoms, which include restlessness, being easily fatigued, difficulty concentrating, irritability, muscle tension, and disturbed sleep. The intensity, duration, or frequency of the anxiety and worry is far out of proportion to the actual likelihood or impact of the fear event. Adults with the disorder often worry about everyday, routine life circumstances such as possible job responsibilities, finances, the health of family members, misfortune of their children, or minor matters. During the course of the disorder, the focus of worry may shift from one concern to another. See DSM-IV-TR, 4^{th} ed., pp. 472-476 (2000).

GAD very frequently co-occurs with Mood Disorders, Anxiety Disorders, or Substance-Related Disorders. The lifetime prevalence rate for GAD is 5%. The criteria for diagnosis for 300.02 Generalized Anxiety Disorder according to the DSM-IV is as follows:
A. Excessive anxiety and worry (apprehensive expectation), occurring more days than not for at least 6 months, about a number of events or activities (such as work or school performance).
B. The person finds it difficult to control the worry.
C. The anxiety and worry are associated with three (or more) of the following six symptoms (with at least some symptoms present for more days than not for the past 6 months).
   1) restlessness or feeling keyed up or on edge
   2) being easily fatigued
   3) difficulty concentrating or mind going blank
   4) irritability
   5) muscle tension
   6) sleep disturbance (difficulty falling or staying asleep, or restless unsatisfying sleep)
D. The focus of the anxiety and worry is not confined to features of an Axis disorder (such as PD, Social Phobia, OCD, Separation Anxiety Disorder, Anorexia Nervosa, Somatization Disorder, Hypochondriasis, or Posttraumatic Stress Disorder).
E. The anxiety, worry, or physical symptoms cause clinically significant distress or impairment in social, occupational, or other important areas of functioning.
F. The disturbance is not due to the direct physiological effects of a substance or general medical condition and does not occur exclusively during a Mood Disorder, a Psychotic Disorder, or a Pervasive Developmental Disorder.

Particular embodiments allow diagnosis and scoring of a simple questionnaire that will permit PCPs to successfully identify and treat most cases of GAD. The patients would then be treated with the compositions described herein including, but not limited to: Li/SSRI; Li/SSRI/Atypical Neuroleptic; and Li/SSRI/Antipsychotic.

### F. Substance Use Disorders

According to the DSM-IV, Substance Use Disorders include Substance Dependence and Substance Abuse. See DSM-IV-TR, 4^{th} ed., pp. 192-199 (2000). Substance Dependence disorder features a cluster of cognitive, behavioral and physiological symptoms indicating continued use of the substance despite significant substance-related problems. There is a pattern of repeated self-administration that can result in tolerance, withdrawal, and compulsive drug-taking behavior. The symptoms of Substance Dependence disorder are similar across various categories of substances (cravings), however, not all symptoms apply to all substances (such as, withdrawal symptoms for hallucinogen dependence).

The incidence of mood disorder in patients diagnosed with SUD approaches 65%. PCPs should assume underlying psychiatric illness whenever they encounter or suspect SUD.

The criteria for Substance Dependence Disorder according to DSM-IV is as follows:

A maladaptive pattern of substance use, leading to clinically significant impairment or distress, as manifested by three (or more) of the following, occurring at any time in the same 12-month period:
(1) tolerance, as defined by either of the following:
   a) a need for markedly increased amounts of the substance to achieve intoxication or desired effect
   b) markedly diminished effect with continued use of the same amount of the substance
(2) withdrawal, as manifested by either of the following:
   a) the characteristic withdrawal syndrome for the substance (See DSM-IV-TR, pp. 201 and 202 for Withdrawal criteria)
   b) the same (or closely related) substance is taken to relieve or avoid withdrawal symptoms
(3) the substance is often taken in larger amounts or over a longer period than was intended
(4) there is a persistent desire or unsuccessful efforts to cut down or control substance use
(5) a great deal of time is spend in activities necessary to obtain the substance, use the substance, or recover from its effects
(6) important social, occupational, or recreational activities are given up or reduced because of substance use
(7) the substance use is continued despite knowledge of having a persistent or recurrent physical or psychological problem that is likely to have been caused or exacerbated by the substance

Specify if:

With Physiological Dependence: evidence of tolerance or withdrawal (either item 1 or 2 present)

Without Physiological Dependence: no evidence of tolerance or withdrawal (neither item 1 nor 2 is present)

Course specifiers: (1) early full remission; (2) early partial remission; (3) sustained full remission; (4) sustained partial remission; (5) on agonist therapy; (6) in a controlled environment. See DSM-IV-TR, 4^{th} ed., pp. 192-199 (2000).

The second Substance Use Disorder is Substance Abuse Disorder, which features a maladaptive pattern of substance use manifested by recurrent and significant adverse consequences related to the repeated use of substances. The criteria for Substance Abuse Disorder as characterized by DSM-IV as follows:
A. A maladaptive pattern of substance use leading to clinically significant impairment or distress, as manifested by one (or more) of the following, occurring within a 12-month period:
   1) recurrent substance use resulting in a failure to fulfill major role obligations at work, school, or home (such as, repeated absences or poor work performance related to substance use; substance-related absences, suspensions, or expulsions from school; neglect of children or household)
   2) recurrent substance use in situations in which it is physically hazardous (such as driving an automobile or operating a machine when impaired by substance use)
   3) recurrent substance-related legal problems (such as arrests for substance-related disorderly conduct)
   4) continued substance use despite having persistent or recurrent social or interpersonal problems caused or exacerbated by the effects of the substance (such as arguments with spouse about consequences of intoxication, physical fights)
B. The symptoms have never met the criteria for Substance Dependence for this class of substance.

Particular embodiments described herein allows diagnosis and scoring of a simple questionnaire that will permit PCPs to successfully identify and treat most cases of SUD. The patients would then be treated with the compositions described herein including, but not limited to: Li/Bupropion; Li/Bupropion/Atypical Neuroleptic; Li/Bupropion/Antipsychotic; Li/Bupropion/Antiseizure; Li/SSRI; Li/SSRI/Antipsychotic; Li/SSRI/Antiseizure; Li/SSRI/Atypical Neuroleptic; Li/SSRI/Antiseizure/Atypical Neuroleptic; Li/SSRI/Tricyclic Antidepressant; Li/MAOI; Li/Atypical Neuroleptic; Bupropion/Tricyclic Antidepressant; Bupropion/Tetracyclic Antidepressant; Li/Antipsychotic; Li/Bupropion/Modafinil; Li/Bupropion/Lamotrigine; Bupropion/Lamotrigine; Li/Mirtazapine.

### G. Pre-Menstrual Dysphoric Disorder

The DSM-IV describes PMDD as featuring markedly depressed mood, marked anxiety, marked affective lability, and decreased interest in activities. These symptoms would regularly occur during the last week of the luteal phase in most menstrual cycles during the past year, remit within a few days of the onset of menses and always be absent in the week following menses. The pattern of symptoms must have occurred most months for the previous 12 months. The most typical pattern appears to be that of dysfunction during the week prior to menses that ends mid-menses. See DSM-IV-TR, 4^{th} ed., pp. 771-774 (2000).

Research criteria for PMDD according to the DSM-TV (See DSM-IV-TR, 4^{th} ed., pp. 771-774 (2000)) include the following:
A. In most menstrual cycles during the past year, five (or more) of the following symptoms were present for most of the time during the last week of the luteal phase, began to remit within a few days after the onset of the follicular phase, and were absent in the week post menses, with at least one of the symptoms being either (1), (2), (3), or (4):
   1) markedly depressed mood, feelings of hopelessness, or self-deprecating thoughts
   2) marked anxiety, tension, feelings of being “keyed up” or “on edge”
   3) marked affective lability (such as feeling suddenly sad or tearful or increased sensitivity to rejection)
   4) persistent and marked anger or irritability or increased interpersonal conflicts
   5) decreased interest in usual activities (such as work, school, friends, hobbies)
   6) subjective sense of difficulty concentrating
   7) lethargy, easy fatigability, or marked lack of energy
   8) marked change in appetite, overeating, or specific food cravings
   9) hypersomnia or insomnia
   10) a subjective sense of being overwhelmed or out of control
   11) other physical symptoms, such as breast tenderness or swelling, headaches, joint or muscle pain, a sensation of “bloating,” weight gain
B. The disturbance markedly interferes with work or school or with usual social activities and relationships with others (such as, avoidance of social activities, decreased productivity and efficiency at work or school).
C. The disturbance is not merely an exacerbation of the symptoms of another disorder, such as MDD, PD, Dysthymia, or Personality Disorder (although it may be superimposed on any of these disorders).
D. Criteria A, B and C must be confirmed by prospective daily ratings during at least two consecutive symptomatic cycles. (The diagnosis may be made provisionally prior to this confirmation.)

Particular embodiments allow diagnosis and scoring of a simple questionnaire that will permit PCPs to successfully identify and treat most cases of PMDD. The patients would then be treated with the compositions described herein including, but not limited to: Li/SSRI; Li/SSRI/Antiseizure; Li/SSRI/Atypical Neuroleptic; Li/Antiseizure; Li/Atypical Neuroleptic; and Li/Bupropion.

### H. Panic Disorder

According to the DSM-IV, Panic Disorder is a type of Anxiety Disorder which is featured by the presence of recurrent, unexpected panic attacks followed by at least 1 month of persistent concern about having another panic attack, worry about the possible implications or consequences of the panic attack, or significant behavioral change related to the attack. Panic Attacks are not due to physiological effects of a substance or general medical condition.

Panic “attacks” manifest as discrete episodes lasting 20 minutes to a few hours. During the panic attack, patients often experience shortness of breath, chest pains, fear of heart attack, sweating, shaking, fears of dying, fear of losing control and going crazy. (Kaplan and Sadock, 7^{th} Ed. p. 585).

The frequency and severity of panic attacks vary widely. In addition to worry about panic attacks, many individuals also report constant or intermittent feelings of anxiety that are not focused on any specific situation or event. Reported rates for comorbid Major Depressive Disorder vary widely, ranging from 10% to 65% in individuals with Panic Disorder. In about a third of individuals with both disorders, the depression precedes the Panic Disorder. In the remaining two thirds, depression occurs coincident with or following the onset of the Panic Disorder. Prevalence of Panic Disorder has been reported as high as 3.5% of the population, however, most studies have found rates between 1-2%. Prevalence rates are higher, approximately 10%, for those referred for mental consultation. See DSM-IV-TR, 4^{th} ed., pp. 433-440 (2000).

The diagnostic criteria for 300.01 Panic Disorder without Agoraphobia is as follows
A. Both (1) and (2):
   (1) recurrent unexpected Panic Attacks (See DSM-IV-TR, 4^{th} ed., p. 432 (2000) for criteria for Panic Attack)
   (2) at least one of the attacks has been followed by 1 month (or more) of one (or more) of the following:
      a) persistent concern about having additional attacks
      b) worry about the implication of the attack or its consequences ( such as, losing control, having a heart attack, “going crazy”)
      c) a significant change in behavior related to the attacks
B. Absence of Agoraphobia (See DSM-IV-TR, 4^{th} ed., p. 433 (2000) for criteria for Agoraphobia.)
C. The Panic Attacks are not due to the direct physiological effects of a substance (such as, drug abuse, a medication) or a general medical condition (such as, hyperthyroidism)
D. The Panic Attacks are not better accounted for by another mental disorder, such as Social Phobia, Specific Phobia, Post-Traumatic Stress Disorder, or Separation Anxiety Disorder

Particular embodiments allow diagnosis and scoring of a simple questionnaire that will permit PCPs to successfully identify and treat most cases of PD. The patients would then be treated with the compositions described herein including, but not limited to, Li/SSRI and Lithium/SSRI/Atypical Neuroleptic.

### I. Posttraumatic Stress Disorder

The essential features of PTSD is the development of characteristic symptoms following exposure to an extreme traumatic stressor involving direct personal experience of an event that involves actual or threatened death or serious injury, or other threat to one's physical integrity; or witnessing an event that involves death, injury, or a threat to the physical integrity of another person; or learning about unexpected or violent death, serious harm, or threat of death or injury experienced by a family member or other close associate. See DSM-IV-TR, 4^{th} ed., pp. 463-468 (2000). The person's response to the event must involve intense fear, helplessness, or horror. Symptoms resulting from the exposure include persistent re-experiencing of the traumatic event, persistent avoidance of stimuli associated with the trauma and numbing of general responsiveness, and persistent symptoms of increased arousal. The symptoms must be present for more than 1 month and the disturbance must cause clinically significant distress or impairment in social, occupational, or other important areas of functioning. See DSM-IV-TR, 4^{th} ed., pp. 463-468 (2000).

Symptoms of PTSD usually begin within the first three months after the trauma, although a delay of months or even years is not uncommon. Duration of the symptoms varies, with complete recovery occurring within 3 months in approximately half of the cases, with many others having persisting symptoms for longer than 12 months after the trauma. In some cases, the course is characterized by waxing and waning of symptoms. See DSM-IV-TR, 4^{th} ed., pp. 463-468 (2000).

The diagnostic criteria for 309.81 PTSD according to DSM-IV is as follows:
A. The person has been exposed to a traumatic event in which both of the following were present:
   (1) the person experienced, witnessed, or was confronted with an event or events that involved actual or threatened death or serious injury, or a threat to the physical integrity of self or others
   (2) the person's response involved intense fear, helplessness, or horror.
B. The traumatic even is persistently re-experienced in one (or more) of the following ways:
   (1) recurrent and intrusive distressing recollections of the event, including images, thoughts ore perceptions.
   (2) recurrent distressing dreams of the event
   (3) acting or feeling as if the traumatic event were recurring (includes a sense of reliving the experience, illusions, hallucinations, and dissociative flashback episodes, including those that occur on awakening or when intoxicated)
   (4) intense psychological distress at exposure to internal or external cues that symbolize or resemble an aspect of the traumatic event
   (5) physiological reactivity on exposure to internal or external cues that symbolize or resemble an aspect of the traumatic event
C. Persistent avoidance of stimuli associated with the trauma and numbing of general responsiveness (not present before the trauma), as indicated by three (or more) of the following:
   (1) efforts to avoid thoughts, feelings, or conversations associated with the trauma
   (2) efforts to avoid activities, places, or people that arouse recollections of the trauma
   (3) inability to recall an important aspect of the trauma
   (4) markedly diminished interest or participation in significant activities
   (5) feeling of detachment or estrangement from others
   (6) restricted range of affect (such as being unable to have loving feelings)
   (7) sense of foreshortened future (such as not expecting to have a career, marriage, children, or a normal life span)
D. Persistent symptoms of increased arousal (not present before the trauma), as indicated by two (or more) of the following:
   (1) difficulty falling or staying asleep
   (2) irritability or outbursts of anger
   (3) difficulty concentrating
   (4) hypervigilance
   (5) exaggerated startle response
E. Duration of the disturbance (symptoms in Criteria B, C, and D) is more than 1 month.
F. The disturbance causes clinically significant distress or impairment in social, occupational, or other important areas of functioning.

Specify if:

Acute: if duration of symptoms is less than 3 months

Chronic: if duration of symptoms is 3 months or more

Specify if:

With Delayed Onset: if onset of symptoms is at least 6 months after the stressor

Particular embodiments allow diagnosis and scoring of a simple questionnaire that will permit PCPs to successfully identify and treat most cases of PTSD. The patients would then be treated with the compositions described herein including, but not limited to: Li/SSRI; Li/SSRI/Atypical Neuroleptic; and Li/Antiseizure.

### J. Social Anxiety Disorder or Social Phobia

Social Phobia is characterized by marked and persistent fear of social or performance situations in which embarrassment may occur. Exposure to a social or performance situation almost invariably provokes an immediate anxiety response. This response may take the form of a situationally bound or situationally predisposed Panic Attack. Sufferers recognize that their fear is excessive or unreasonable. Most often, the social or performance situation is avoided, although sometimes it is endured with dread. The diagnosis is appropriate only if the avoidance, fear, or anxious anticipation of encountering the social or performance situation interferes significantly with the person's daily routine, occupational functioning, or social life, or if the person is markedly distressed abut having the phobia. See DSM-IV-TR, 4^{th} ed., pp. 450-456 (2000).

Social Phobia may be associated with other Anxiety Disorders, Mood Disorders, Substance-Related Disorders, and Bulimia Nervosa and usually precedes these disorders. Epidemiological and community-based studies have reported a lifetime prevalence of Social Phobia ranging from 3-13%. Onset is usually in the mid-teens, although some report onset in early childhood. See DSM-IV-TR, 4^{th} ed., pp. 450-456 (2000).

According to the DSM-IV, diagnostic criteria for 300.23 Social Phobia include:
A. A marked and persistent fear of one or more social or performance situations in which the person is exposed to unfamiliar people or to possible scrutiny by others. The individual fears that he or she will act in a way (or show anxiety symptoms) that will be humiliating or embarrassing.
B. Exposure to the feared social situation almost invariably provokes anxiety, which may take the form of a situationally bound or situationally predisposed Panic Attack.
C. The person recognizes that the fear is excessive.
D. The feared social or performance situations are avoided or else are endured with intense anxiety or distress.
E. The avoidance, anxious anticipation, or distress in the feared social or performance situation(s) interferes significantly with the person's normal routine, occupational (academic) functioning, or social activities or relationships, or there is marked distress about having the phobia.
F. In individuals under age 18 years, the duration is at least 6 months.
G. The fear or avoidance is not due to the direct physiological effects of a substance (such as, a drug of abuse, a medication) or a general medical condition and is not better accounted for by another mental disorder (such as, Panic Disorder with or without Agoraphobia, Separation Anxiety Disorder, Body Dysmorphic Disorder, a Pervasive Developmental Disorder, or Schizoid Personality Disorder).
H. If a general medical condition or another mental disorder is present, the fear in Criterion A is unrelated to it, (such as, the fear is not Stuttering, trembling in Parkinson's disease, or exhibiting abnormal eating behavior in Anorexia Nervosa or Bulimia Nervosa)

Specify if:

Generalized: if the fears include most social situations (also consider the additional diagnosis of Avoidant Personality Disorder).

Particular embodiments allow diagnosis and scoring of a simple questionnaire that will permit PCPs to successfully identify and treat most cases of SAD. The patients would then be treated with the compositions described herein including, but not limited to, Li/SSRI; Li/SSRI/Atypical Neuroleptic; and Li/SSRI/Antipsychotic.

### K Borderline Personality Disorder

Borderline personality disorder usually presents with a pervasive pattern of instability of self-image, interpersonal relationships and mood. BPD can begin in early adulthood. BPD is marked by identity disturbance which can manifest as uncertainty about life issues, self-image, sexual orientation, long-term goals, career choice, types of friends or lovers to have, or which values to adopt. See DSM-IV-TR, 4^{th} ed., p. 706-710 (2000).

Individuals with BPD make frantic efforts to avoid real or imagined abandonment, needing to have other people with them. In addition, BPD sufferers may also exhibit a pattern of unstable and intense relationships, wherein they may idealize potential caregivers or lovers early in the relationship and then switch quickly from idealizing to devaluing them. BPD patients also may show marked or persistently unstable self-image or sense of self. Their self-image dramatically shifts, characterized by shifting goals, values, and vocational aspirations. Individuals with this disorder also display impulsivity in at least two areas that are potentially self damaging, such as, gambling, spending money irresponsibly, binge eating, abuse substances, engage in unsafe sex, or drive recklessly. See DSM-IV-TR, 4^{th} ed., p. 706-710 (2000).

Individuals with BPD also display recurrent suicidal behavior, gestures or threats or self-mutilating behavior. Completed suicide occurs in 8-10% in BPD individuals with self-mutilative acts and suicide attempts being very common. Individuals with this disorder also may display a marked reactivity of mood, such as intensive episodic dysphoria, irritability, or anxiety usually lasting a few hours and only rarely more than a few days. These episodes may reflect the individual's extreme reactivity to interpersonal stresses. Individuals with BPD may also be troubled by chronic feelings of emptiness or be easily bored. Intense anger or difficulty controlling anger is often displayed as extreme sarcasm, enduring bitterness or verbal outbursts by individuals with the disorder. During periods of extreme stress, transient paranoid ideation or dissociative symptoms may occur, but they tend to be transient lasting minutes or hours. See DSM-IV-TR, 4^{th} ed., p. 706-710 (2000).

According to the DSM-IV-TR, borderline personality disorder (301.83) is diagnosed according to the following 9 criteria if a patient presents with 5 or more of the criteria. See DSM-IV-TR, 4^{th} ed., p. 706-710 (2000).
1. frantic efforts to avoid real or imagined abandonment. Note: Do not include suicidal or self-mutilating behavior covered in Criterion 5.
2. a pattern of unstable and intense interpersonal relationships characterized by alternating between extremes of idealization and devaluation
3. identity disturbance: markedly and persistently unstable self-image or sense of self
4. impulsivity in at least two areas that are potentially self-damaging (such as, spending, sex, substance abuse, reckless driving, binge eating). Note: Do not include suicidal or self-mutilating behavior covered in Criterion 5.
5. recurrent suicidal behavior, gestures, or threats, or self-mutilating behavior
6. affective instability due to a marked reactivity of mood ( such as, intense episodic dysphoria, irritability, or anxiety usually lasting a few hours and only rarely more than a few days)
7. chronic feelings of emptiness
8. inappropriate, intense anger or difficulty controlling anger (such as, frequent displays of temper, constant anger, recurrent physical fights)
9. transient, stress-related paranoid ideation or severe dissociative symptoms

Particular embodiments allow diagnosis and scoring of a simple questionnaire that will permit PCPs to successfully identify and treat most cases of BPD. The patients would then be treated with the compositions described herein including, but not limited to: Li/SSRI; Li/SSRI/Atypical Neuroleptic; Li/SSRI/Antipsychotic; Li/SSRI/Antiseizure; Li/Atypical Neuroleptic; Bupropion/Tricyclic Antidepressant; Bupropion/Tetracyclic Antidepressant; Li/Antipsychotic; Li/MAOI; Li/Bupropion/Modafinil; Li/Bupropion/Lamotrigine; Bupropion/Lamotrigine; Li/Bupropion; Li/Mirtazapine.

### L. Schizoaffective Disorder

The essential feature of Schizoaffective Disorder is an uninterrupted period of illness during which, at some time, there is a Major Depressive, Manic, or Mixed Episode concurrent with symptoms that meet with Criterion A for Schizophrenia (See p. 312 of DSM-IV). In addition, during the same period of illness, there have been delusions or hallucinations for at least 2 weeks in the absence of prominent mood symptoms. Finally, the mood symptoms are present for a substantial portion of the total duration of the illness. The essential features must occur within a single uninterrupted period of illness. “Period of illness” refers to a time period during which the individual continues to display active or residual symptoms of psychotic illness. See DSM-IV-TR, 4^{th} ed., p. 319-323 (2000).

Two subtypes of Schizoaffective Disorder may be noted based on the mood component. One subtype is Bipolar Type, this type applies if a Manic Episode, a Mixed Episode or Major Depressive Episode (See DSM-IV-TR, pp. 349-364) is part of the presentation. The other subtype is Depressive Type, this type applies if only Major Depressive Episodes are part of the presentation. See DSM-IV-TR, 4^{th} ed., p. 319-323 (2000).

The diagnostic criteria for 295.70 Schizoaffective Disorder is as follows:
A. An uninterrupted period of illness during which, at some time, there is either a Major Depressive Episode, a Manic Episode, or a Mixed Episode concurrent with symptoms that meet Criterion A for Schizophrenia. Note: The Major Depressive Episode must include Criterion Al: depressed mood.
B. During the same period of illness, there have been delusions or hallucinations for at least 2 weeks in the absence of prominent mood symptoms.
C. Symptoms that meet criteria for a mood episode are present for a substantial portion of the total duration of the active and residual periods of the illness.
D. The disturbance is not due to the direct physiological effects of a substance (such as, a drug of abuse, a medication) or a general medical condition.

Specify type:

Bipolar Type: if the disturbance includes a Manic or Mixed Episode (or a Manic or Mixed Episode and Major Depressive Episodes)

Depressive Type: if the disturbance only includes Major Depressive Episodes

Particular embodiments allow diagnosis and scoring of a simple questionnaire that will permit PCPs to successfully identify and treat most cases of Schizoaffective Disorder.

The patients with Schizoaffective Disorder, depressed type, in the acute and maintenance phases of treatment, would then be treated with the compositions described herein including, but not limited to: Li/Atypical Neuroleptic; Li/Antipsychotic; Li/S SRI/Atypical Neuroleptic; Li/S SRI/Antipsychotic; Li/S SRI/Antipsychotic/Antiseizure; Li/SSRI/Atypical Neuroleptic/Antiseizure; Li/SSRI/Bupropion; Li/SSRI/Bupropion/Atypical Neuroleptic; and Li/SSRI/Bupropion/Antipsychotic.

The patients with Schizoaffective Disorder, manic type, in the acute and maintenance phases of treatment, would then be treated with the compositions described herein including, but not limited to: Li/Atypical Neuroleptic; Li/Antipsychotic; Li/Atypical/Antiseizure; Li/Antipsychotic/Antiseizure; Li/Bupropion/Atypical Neuroleptic; Li/Bupropion/Antipsychotic; Li/Bupropion/Atypical Neuroleptic/Antiseizure; Li/Bupropion/Antipsychotic/Antiseizure; Li/SSRI/Atypical Neuroleptic; Li/SSRI/Antipsychotic; Li/SSRI/Antiseizure; Li/SSRI/Atypical Neuroleptic/Antipsychotic; and Bupropion/Antiseizure.

The patients with Schizoaffective Disorder, mixed type, in the acute and maintenance phases of treatment, would then be treated with the compositions described herein including, but not limited to: Bupropion/Antiseizure drug.

### M. Seasonal Affective Disorder

According the DSM-IV-TR, Seasonal Pattern is a specifier applied to the pattern of Major Depressive Episodes in Bipolar I Disorder, Bipolar II Disorder, or Major Depressive Disorder, Recurrent. The essential feature is the onset and remission of Major Depressive Episodes at characteristic times of year. Most cases have episodes that begin in the fall or winter which remit in spring, however, summer episodes do occur. This pattern of onset and remission must have occurred during the last 2 years, without any nonseasonal episodes occurring during this period. In addition, the number of seasonal depressive episodes must outnumber the any non-seasonal depressive episodes over the lifetime of the individual. See DSM-IV-TR, 4^{th} ed., pp. 425-427 (2000).

The criteria from the DSM-IV-TR for Seasonal Pattern Specifier is as follows:

Specify if:

With Seasonal Pattern (can be applied to the pattern of Major Depressive Episodes in Bipolar I Disorder, Bipolar II Disorder, or Major Depressive Disorder, Recurrent)
A. There has been a regular temporal relationship between the onset of Major Depressive Episodes in Bipolar I or Bipolar II Disorder or Major Depressive Disorder, Recurrent, and a particular time of the year (such as, regular appearance of the Major Depressive Episode in the fall or winter). Note: Do not include cases in which there is an obvious effect of seasonal related psychosocial stressors (such as, regularly being unemployed every winter).
B. Full remissions (or a change from depression to mania or hypomania) also occur at a characteristic time of the year (such as, depression disappears in the spring).
C. In the last 2 years, two Major Depressive Episodes have occurred that demonstrate the temporal seasonal relationships defined in Criteria A and B, and no nonseasonal Major Depressive Episodes have occurred during that same period.
D. Seasonal Major Depressive Episodes (as described above) substantially outnumber the nonseasonal Major Depressive Episodes that may have occurred over the individual's lifetime.

Particular embodiments allow diagnosis and scoring of a simple questionnaire that will permit PCPs to successfully identify and treat most cases of seasonal affective disorder. The patients would then be treated with the compositions described herein including, but not limited to: Li/SSRI; Li/SSRI/Atypical Neuroleptic; Li/SSRI/Antiseizure; Li/Bupropion; Li/Bupropion/Atypical Neuroleptic; and Li/Tricyclic Antidepressant.

### N. Eating Disorders

Eating disorders are characterized by severe disturbances in eating behavior. Two eating disorders include Anorexia and Bulimia. Anorexia Nervosa is characterized by a refusal to maintain a minimally normal body weight. Bulimia Nervosa is characterized by repeated binge eating followed by inappropriate compensatory behaviors such as self-induced vomiting, misuse of laxatives, diuretics, other medications, fasting, or excessive exercise. See DSM-IV-TR, 4^{th} ed., p. 583 (2000).

1. Anorexia Nervosa

Anorexia Nervosa primarily occurs primarily to young females of industrialized societies where there is an abundance of food and being thin is considered attractive. Anorexia presents with the individual refusing to maintain minimally normal body weight, is intensely afraid of gaining weight, and exhibits a significant disturbance in the perception of the shape or size of his or her body. Typically, postmenarcheal females with the disorder are amenorrheic. See DSM-IV-TR, 4^{th} ed., pp. 583-589 (2000).

When seriously underweight, many individuals with Anorexia manifest depressive symptoms such as depressed mood, social withdrawal, irritability, insomnia and diminished interest in sex. Many depressive features may be secondary to the physiological sequelae of semistarvation, therefore, mood disturbance must be reassessed after partial or complete weight restoration. See DSM-IV-TR, 4^{th} ed., pp. 583-589 (2000).

Obsessive-compulsive features, both related and unrelated to food are often prominent. Most afflicted individuals are preoccupied with thoughts of food and may collect recipes or hoard food. When the patient exhibits obsessions and compulsions that are not related to food, body shape, or weight, an additional diagnosis of OCD may be warranted. See DSM-IV-TR, 4^{th} ed., p. 583-589 (2000).

The diagnostic criteria for 307.1 Anorexia Nervosa according to the DSM-IV is as follows:
A. Refusal to maintain body weight at or above a minimally normal weight for age and height (such as weight loss leading to maintenance of body weight less than 85% of that expected).
B. Intense fear of gaining weight or becoming fat, even though underweight.
C. Disturbance in the way in which one's body weight or shape is experienced, undue influence of body weight or shape on self-evaluation, or denial of the seriousness of the current low body weight.
D. In postmenarcheal females, amenorrhea, i.e., the absence of at least three consecutive menstrual cycles (A woman is considered to have amenorrhea if her periods occur only following hormone, e.g. estrogen administration.)

Specify type:

Restricting Type: during the current episode of Anorexia Nervosa, the person has not regularly engaged in binge-eating or purging behavior (such as, self-induced vomiting or the misuse of laxatives, diuretics, or enemas).

Binge-Eating/Purging Type: during the current episode of Anorexia Nervosa, the person has regularly engaged in binge-eating or purging behavior (such as, self-induced vomiting or the misuse of laxatives, diuretics, or enemas).

2. Bulimia Nervosa

The key features of Bulimia Nervosa are binge eating and inappropriate compensatory methods to prevent weight gain. The self-evaluation of individuals with Bulimia Nervosa is excessively influenced by body shape and weight. A binge is defined as eating in a discrete period of time an amount of food that is definitely larger than most individuals would eat under similar circumstances. A “discrete period of time” refers to a limited period, usually less than 2 hours. A single episode of binge eating need not be restricted to one setting. An individual, for example, could begin a binge in a restaurant and then continue it on returning home. Continual snacking on small amounts of food throughout the day would not be considered a binge. See DSM-IV-TR, 4^{th} ed., p. 589-594 (2000).

Individuals with Bulimia have an increased frequency of depressive symptoms or Mood Disorders. The mood disturbance typically begins at the same time as or following the development of Bulimia Nervosa, and the individuals often ascribe their mood disturbance to the Bulimia. In some individuals, however, the mood disturbance precedes the development. There may also be an increase in the frequency of anxiety symptoms or Anxiety Disorders. See DSM-IV-TR, 4^{th} ed., p. 589-594 (2000).

The diagnostic criteria for 307.51 Bulimia Nervosa according to the DSM-IV is as follows:
A. Recurrent episodes of binge eating. An episode of binge eating is characterized by both of the following:
   (1) eating, in a discrete period of time, an amount of food that is definitely larger than most people would eat during a similar period of time and under similar circumstances
   (2) a sense of lack of control over eating during the episode (such as, feeling that one cannot stop eating or control what or how much one is eating)
B. Recurrent inappropriate compensatory behavior in order to prevent weight gain, such as self-induced vomiting; misuse of laxatives, diuretics, enemas, or other medications; fasting; or excessive exercise.
C. The binge eating and inappropriate compensatory behaviors both occur, on average, at least twice a week for 3 months.
D. Self-evaluation is unduly influenced by body shape and weight.

E. The disturbance does not occur exclusively during episodes of Anorexia Nervosa.

Specify type:

Purging Type: during the current episode of Bulimia Nervosa, the person has regularly engaged in self-induced vomiting or the misuse of laxatives, diuretics, or enemas

Nonpurging Type: during the current episode of Bulimia Nervosa, the person has used other inappropriate compensatory behaviors, such as fasting or excessive exercise, but has not regularly engaged in self-induced vomiting or the misuse of laxatives, diuretics, or enemas.

Particular embodiments allow diagnosis and scoring of a simple questionnaire that will permit PCPs to successfully identify and treat most cases of eating disorders. The patients would then be treated with the compositions described herein including, but not limited to: Li/SSRI; Li/SSRI/Atypical Neuroleptic; Li/SSRI/Atypical Neuroleptic/Antiseizure; Li/Atypical Neuroleptic; Li/Antipsychotic; Li/Tricyclic Antidepressant; and Li/Antiseizure.

### O. Depressive Personality Disorder

Depressive Personality Disorder is characterized by a pervasive pattern of depressive cognition and behavior that begins by early adulthood. The behaviors and cognitions include a persistent and pervasive feeling of dejection, gloominess, cheerlessness, joylessness, and unhappiness. Individuals with DPD tend to have low self-esteem and are particularly focused on feelings of inadequacy. They also may be critical and judgmental toward others. See DSM-IV-TR, 4^{th} ed., p. 788 (2000).

Individuals with Depressive Personality Disorder may be predisposed to developing Dysthymic Disorder and possibly Major Depressive Disorder. Additionally, preliminary evidence suggests that depressive personality disorder may have an increased prevalence in family members of probands with Major Depressive Disorder.

Diagnostic criteria for DPD disorder according to the DSM-IV (See DSM-IV-TR, 4^{th} ed., pp. 788-789 (2000)) include the following:
A. A pervasive pattern of depressive cognitions and behaviors beginning by early adulthood and present in a variety of contexts, as indicated by five (or more of the following:
   1) usual mood is dominated by dejection, gloominess, cheerlessness, joylessness, unhappiness
   2) self-concept centers around beliefs of inadequacy, worthlessness, and low self-esteem
   3) is critical, blaming, and derogatory toward self
   4) is brooding and given to worry
   5) is negativistic, critical, and judgmental toward others
   6) is pessimistic
   7) is prone to feeling guilty or remorseful
B. Does not occur exclusively during Major Depressive Episodes and is not better accounted for by Dysthymic Disorder.

Particular embodiments described herein allows diagnosis and scoring of a simple questionnaire that will permit PCPs to successfully identify and treat most cases of depressive personality disorder. The patients would then be treated with the compositions described herein including, but not limited to: Li/SSRI; Li/SSRI/Antiseizure; Li/SSRI/Bupropion; Li/SSRI/Bupropion/Antiseizure; Bupropion/Tricyclic Antidepressant; Bupropion/Tetracyclic Antidepressant; Li/Antipsychotic; Li/MAOI; Li/Bupropion/Modafinil; Li/Bupropion/Lamotrigine; Bupropion/Lamotrigine; Li/Bupropion; and Li/Mirtazapine.

### P. Cyclothymic Disorder

According to the DSM-IV-TR, Cyclothymic disorder is characterized by a chronic, fluctuating mood disturbance involving periods of hypomanic symptoms and numerous periods of depressive symptoms. The hypomanic symptoms, however. are insufficient in terms of number, severity, pervasiveness, or duration to meet with the full criteria for a Manic Episode. The depressive symptoms are similarly insufficient, in that the number, severity, pervasiveness or duration, are also insufficient to meet with the full criteria for a Major Depressive Episode. The diagnosis of Cyclothymic Disorder is made only if the initial 2-year period of cyclothymic symptoms is free of Major Depressive, Manic and Mixed Episodes with any symptom-free periods not lasting longer than 2 months. After the initial 2 years of Cyclothymic Disorder, Manic or Mixed Episodes may be superimposed on the Cyclothymic Disorder, in which case both Cyclothymic and Bipolar I Disorders are diagnosed. Alternatively, if Major Depressive Episodes are superimposed on the Cyclothymic Disorder after the initial 2 years, then Cyclothymic Disorder is diagnosed with Biopolar II Disorder. See DSM-IV-TR, 4^{th} ed., p. 398 (2000).

Cyclothymic Disorder (“CD”) often begins early in life and is sometimes considered to reflect a temperamental predisposition to other Mood Disorders. CD is apparently common in men and women with a lifetime prevalence from 0.4% to 1%. CD usually begins in adolescence or early adult life. Onset of CD late in life may suggest Mood Disorder Due to a General Medical Condition such as multiple sclerosis. See DSM-IV-TR, 4^{th} ed., p. 398-399 (2000).

Diagnostic criteria for CD disorder according to the DSM-IV (>See DSM-IV-TR, 4th ed., pp. 400(2000)) include the following:
A. For at least 2 years, the presence of numerous periods with hypomanic symptoms and numerous periods with depressive symptoms that do not meet criteria for a Major Depressive Episode. In children and adolescents, the duration must be at least a year.
B. During the above 2-year period (1year for children and adolescents), the person has not been without the symptoms in Criterion A for more than 2 months at a time.
C. No Major Depressive Episode, Manic Episode, or Mixed Episode has been present during the first 2 years of the disturbance. After the initial 2 years (1 year in children and adolescents) of Cyclothymic Disorder, there may be superimposed Manic or Mixed Episodes (in which case both Bipolar I Disorder and Cyclothymic Disorder may be diagnosed) or Major Depressive Episodes (in which case both Bipolar II Disorder and Cyclothymic Disorder may be diagnosed).
D. The symptoms in Criterion A are not better accounted for by Schizoaffective Disorder and are not superimposed on Schizophrenia, Schizophreniform Disorder, Delusional Disorder, or Psychotic Disorder Not Otherwise Specified.
E. The symptoms are not due to the direct physiological effects of substance (for example, a drug of abuse, a medication) or a general medical condition ( such as hyperthyroidism).
F. The symptoms cause clinically significant distress or impairment in social, occupational, or other important areas of functioning.

Particular embodiments described herein allows diagnosis and scoring of a simple questionnaire that will permit PCPs to successfully identify and treat most cases of CD. The patients would then be treated with the compositions described herein including, but not limited to: Li/SSRI; Li/SSRI/Atypical Neuroleptic; Li/SSRI/Antipsychotic; Li/SSRI/Antiseizure; Li/Atypical Neuroleptic, Bupropion/Tricyclic Antidepressant; Bupropion/Tetracyclic Antidepressant; Li/Antipsychotic; Li/MAOI; Li/Bupropion/Modafinil; Li/Bupropion/Lamotrigine; Bupropion/Lamotrigine; Li/Bupropion; and Li/Mirtazapine.

### III. Pharmaceutical Compounds of The Invention: Lithium, SSRIs, Antiseizure Drugs, Atypical Neuroleptics, Antipsychotics, Tricyclic Antidepressants, MAOI's, And Others

### A. Lithium

Lithium is a monovalent cation which belongs to the group of alkali metals together with sodium, potassium and other elements with which it shares some of its properties. Unlike other antimanic agents, it does not possess general sedative properties. Lithium enhances the uptake of norepinephrine and serotonin into the synaptosomes, thus reducing their action. It reduces release of norepinephrine from synaptic vesicles and inhibits production of cyclic AMP.

Lithium ions are rapidly absorbed from the gastrointestinal tract and plasma lithium peaks are reached 2 to 4 hours after lithium administration. The distribution of lithium in the body approximates that of total body water, but its passage across the blood-brain barrier is slow and at equilibration the CSF lithium level reaches only approximately half the plasma concentration.

Approximately 95% of a single dose of lithium is eliminated in urine. The elimination half-life of lithium averages 20 to 24 hours. Half-life in geriatric patients and patients with impaired renal function is increased. With repeated administration of lithium, excretion increases during the first five to six days until a steady state is reached between ingestion and excretion.

Lithium is excreted primarily in urine with less than 1% being eliminated with the feces. Lithium is filtered by the glomeruli and 80% of the filtered lithium is reabsorbed in the tubules. The renal clearance of lithium is proportional to its plasma concentration. About 50% of a single dose of lithium is excreted in 24 hours.

Renal lithium clearance is, under ordinary circumstances, remarkably constant in the same individual, but decreases with age and falls when sodium intake is lowered. The dose necessary to maintain a given concentration of serum lithium depends on the ability of the kidney to excrete lithium. Renal lithium excretion, however, may vary greatly between individuals and lithium dosage should be adjusted individually. In clinical reports, it has been noted that serum lithium may rise an average of 0.2 to 0.4 mmol/L after intake of 300 mg and 0.3 to 0.6 mmol/L after intake of 600 mg of lithium carbonate. It has been suggested that manic patients retain larger amounts of lithium during the active manic phase, but recent studies have been unable to confirm a clear difference in excretion patterns. However, patients in a manic state seem to have an increased tolerance to lithium.

Since 1972, lithium is the most widely used therapy for bipolar disorder. The FDA has approved lithium's use for both acute mania and relapse prevention. Lithium decreases both the frequency and intensity of recurrence of mood disorder. This attribute of lithium is immensely important because the average number of recurrent episodes, for example, manic depression (bipolar mood disorder), is greater than eight per lifetime. Lithium has a fifty-year track record in psychiatry. Of all the currently available mood stabilizers, lithium is the most efficacious mood stabilizer for chronic stabilization of both acute and maintenance treatment of mood spectrum disorders. Those skilled in the art of psychiatry, however, teach away from the use of lithium citing lithium's narrow therapeutic range. Over the past ten years, lithium has been replaced by a number of anti-convulsants, such as valproic acid, topiramate and lamotrigine. Of these antiseizure medications only lamotrigine has received maintenance indications in treating both the depressed and manic phases of bipolar disorder. The newer anti-convulsant class of mood stabilizers has been portrayed as potentially having less long-term side effects, while, at the same time, as being more effective than lithium.

Treatment with these newer agents, however, can cost in excess of ten dollars a day, while treatment with lithium costs less than twenty-five cents a day. Nevertheless, many current academic research and clinical psychiatrists specializing in the treatment of mood disorders continue to value lithium as the “gold standard” of treatment. For example, a recent study comparing lamotrigine (LAMICTAL) to lithium in the maintenance treatment of bipolar patients over a period of eighteen months concludes, “Lamotrigine and lithium were superior to placebo for the prevention of mood episodes in bipolar I patients, with lamotrigine predominantly effective against depression and lithium predominantly effective against mania.” Goodwin, et al., J. Clin Psychiatry, 65:432 (2004).

Lithium is approved for the treatment of: schizo-affective disorder, depressed, manic and mixed types; bipolar I, depressed, manic, and mixed types and bipolar II. Additional “off label” uses for lithium include as an augmenting agent in combination with antidepressants in treating both acute and maintenance phases of MDD, SUD, impulse-control disorder, dysthymia, treatment resistant or atypical depressions and the cyclothymia associated with Borderline Personality Disorder.

Concentrations considered to be effective and acceptably safe are between 0.60 and 1.25 mEq per liter; the range of 0.9 to 1.1 mEq per liter is favored for treatment of acutely manic or hypomanic patients. For long-term maintenance use, 0.6 to 1.2 mEq per liter are considered adequate. These concentrations refer to serum or plasma samples obtained at 10±2 hours after the last oral dose of the day. The recommended concentration is usually attained by doses of 900 to 1500 mg of lithium carbonate per day in outpatients; the optimal dose tends to be larger in younger and heavier patients.

The extended-release form of lithium decreases acute side effects and enhances drug tolerability. Acute gastrointestinal symptoms are significantly lessened by preventing high peak blood levels. Long-term side effects can be diminished by keeping lithium blood levels at the 0.6 mM/mil level, which has been found to be the critical level needed to prevent recurrences. Long-term side effects can further be lessened by assessing bi-annual blood work and EKGs. By establishing baseline measures and carefully monitoring treatment side effects along with lithium blood levels, BUN, creatinine, electrolytes, T3, T4, TSH, individualized clinical decisions can make treatment with the invention both safe and efficacious.

Lithium has long been recognized to have unique anti-suicidal effects. With the lifetime prevalence of completed suicide among bipolar patients approaching twenty percent, lithium's anti-suicidal properties would save countless lives if used in the maintenance treatment of mood disorders. Further, the addition of lithium to an antidepressant is a powerfully synergistic technique in successfully managing treatment-resistant depression, dysthymia and bipolar disorder.

In particular embodiments, it is contemplated that lithium is combined with either SSRIs, antiseizure drugs, atypical neuroleptics, antipsychotics, MAOI's, or tricyclic antidepressants. It is also contemplated that in particular embodiments the single dose formulation of lithium with an SSRI would be combined with either an antiseizure, atypical neuroleptic, or antipsychotic drug as provided herein. In yet other embodiments, it is also contemplated that lithium is combined with bupropion, mirtazapine, or venlafaxine. It is also contemplated that in particular embodiments, lithium is combined with bupropion and lamotrigine in a single formulation. It is further contemplated that lithium may be combined with lamotrigine in a single formulation. In another embodiment, lithium is combined with bupropion and modafinil in a single formulation.

### B. Selective Serotonin Reuptake Inhibitors (SSRIs)

Selective serotonin reuptake inhibitors have been used to treat depression since the mid 1980's. SSRIs were the first class of psychiatric medications to be developed based on molecular targeting. Mourilhe and Stokes, Drug Safety, 18:57-82 (1998). SSRIs possess four major advances over older antidepressants. One, SSRIs are better tolerated by patients with fewer side effects. Two, SSRIs are more easily administered using a once daily dosage with minimal titration to reach therapeutic levels. Three, SSRIs have fewer drug interactions. And four, SSRIs are better tolerated in overdose. Mourilhe and Stokes, Drug Safety, 18:57-82 (1998).

The benign tolerability profile of SSRIs makes SSRIs particularly suitable as the second component to compositions provided herein which are intended for chronic maintenance indications. Therefore, patients in need of long term maintenance medication would benefit from the compositions of the invention.

1. Citalopram

Citalopram, (±)-1-[3-(dimethylamino)propyl]-1-(4-fluorophenyl)-1,3-dihydro-5-isobenzofuran carbonitrile, hydrobromide is disclosed in U.S. Pat. No. 4,136,193 as a serotonin reuptake inhibitor. Citalopram is commercially available as CELEXA (Forest Labs). Its pharmacology was disclosed by Christensen et al., Eur. J. Pharmacol. 41, 153 (1977), and reports of its clinical effectiveness in depression may be found in Dufour et al., Int. Clin. Psychopharmacol. 2:225 (1987), and Timmerman et al., Int. Clin. Psychopharmacol. 2:239-53 (1987).

Citalopram is a selective serotonin reuptake inhibitor (“SSRI”) that is very well tolerated and highly effective. It has relatively few side effects and virtually no drug-drug interactions. Citalopram does not interact with other antidepressants, blood pressure medications, benzodiazepines, antibiotics, prednisone, caffeine, estrogen, acetaminophen, ibuprofen, naproxen, diabetic medication and blood thinners. Citalopram is rarely associated with CNS sedation or agitation. Citalopram has no or very low affinity for 5-HT_{1A}, 5-HT_{2A}, dopamine D₁, D₂, α₁-, αa₂-, and β-adrenergic, histamine H₁, gamma aminobutyric acid, muscarinic cholinergic, and benzodiazepine receptors. There are no vital sign changes in blood pressure or EKG associated with citalopram. Citalopram has an FDA indication for treatment of MDD. It has a rapid onset of action and diminishes both anxiety and depression within one to two weeks.

“Off label” clinical experience has shown citalopram to be particularly useful for the treatment of: generalized anxiety disorder (“GAD”), panic disorder (“PD”), social anxiety disorder (“SAD”), phobia, post-traumatic stress disorder (“PTSD”), dysthymia, obsessive-compulsive disorder (“OCD”), borderline personality disorder (“BPD”), eating disorders, seasonal affective disorder, pre-menstrual dysphoric disorder (“PMDD”) and substance use disorder (“SUD”). For example, with regard to SUDs, among women with alcohol dependence, 60.7% had an anxiety disorder, while 48.5% had depression. Further, among adults with anxiety disorders, 23.7% had a substance use disorder while 32% of all depressives had SUDs.

Single and multiple dose pharmacokinetics of citalopram are linear and dose-proportional in a dose range of 10-60 mg/day. Dose ranges for citalopram can range from 10-360 mg/day. Citalopram is hepatically biotransformed with an average half-life of about 35 hours. Steady state plasma concentrations can be reached within a week with daily dosing. Peak blood levels of citalopram follow four hours after a single 40 mg oral dose. The half life of citalopram is increased 23-50% in elderly patients.

Particular embodiments include citalopram as the second component in combination with a mood stabilizer, such as lithium, in a single formulation. In other embodiments citalopram is administered in combination with lithium and a third component. The third component may be, but not limited to antiseizure drugs, atypical neuroleptics, and antipsychotic drugs as provided herein. These embodiments would be useful for treatment of patients with mood disorder. For example, the combination of lithium and citalopram in a single dosage formulation can be used to treat a patient suffering from mood disorder. A mood disorder includes, but is not limited to, dysthymia, bipolar disorder and treatment resistant depression.

In the instant example, wherein the drug combination is lithium with citalopram, the utilization of two generic drugs can decrease the average daily cost of medication by at least 75 percent. Such dramatic cost savings are particularly significant given that many patients need to take their medications throughout their lives on a chronic maintenance basis.

2. Escitalopram

Escitalopram, S-(+)-1-[3-(dimethylamino)propyl]-1-(p-fluorophenyl)-5-phthalancarbonitrile oxalate, is disclosed in U.S. Pat. No. RE34,712 as the pure S-enantiomer of citalopram. Escitalopram is commercially available as LEXAPRO (Forest Labs).

Generally, 40 mg of citalopram is equivalent to IO mg of escitalopram in the average adult patient. Escitalopram is a highly selective serotonin reuptake inhibitor with minimal effects on norepinephrine and dopamine neuronal reuptake. Escitalopram is at least 100 fold more potent than its R-enantiomer as a serotonin reuptake inhibitor. Escitalopram has no or very low affinity for serotonergic (5-HT₁₋₇) or other receptors including α-adrenergic, β-adrenergic, dopamine (D-₁₋₅), histamine (H₁₋₃), muscarinic (M₁₋₅), and benzodiazepine receptors.

The single- and multiple-dose pharmacokinetics of escitalopram are linear and dose-proportional in a range of 10-30 mg/day. Escitalopram is hepatically biotransformed and has an average half-life of about 27-32 hours. This half life is increased by approximately 50% in elderly patients. Steady state plasma concentrations can be achieved within a week with daily dosing. Following a 20 mg dose, peak blood levels occur at about 5 hours. The binding of escitalopram to human plasma proteins is approximately 56%.

Escitalopram is indicated for MDD and Generalized Anxiety Disorder (GAD) and contraindicated for use with Monoamine Oxidase Inhibitors (“MAOI's”). With respect to particular embodiments of the present invention, it is contemplated that escitalopram would be combined with lithium in a single dosage formulation for treatment of mood disorder. Examples of mood disorder treated by this particular combination include, but is not limited to dysthymia, bipolar depression and treatment resistant depression.

Particular embodiments described herein include escitalopram as the second component in combination with a mood stabilizer, such as lithium, in a single formulation. In other embodiments, escitalopram is administered in combination with lithium and a third component. The third component may be, but not limited to antiseizure drugs, atypical neuroleptics, and antipsychotic drugs as provided herein.

3. Paroxetine

Paroxetine is the hydrochloride salt of a phenylpiperidine compound identified chemically as (Immediate-Release Tablets and Oral Suspension): (−)-trans-4R-(4′-fluorophenyl)-3S-[(3′,4′-methylenedioxyphenloxy) methyl]piperidine hydrochloride hemihydrate and (Controlled-Release Tablets): (−)-(3S,4R)-4-[(p-fluorophenyl)-3-[(3,4-methylenedioxy) phenoxy]methyl]piperidine hydrochloride hemihydrate. Paroxetine is described in U.S. Pat. Nos. 3,912,743 and 4,007,196. Reports of the drug's activity are in Lassen, Eur. J. Pharmacol. 47:351 (1978); Hassan et al., Brit. J. Clin. Pharmacol. 19:705 (1985); Laursen et al., Acta Psychiat. Scand. 71:249 (1985); and Battegay et al., Neuropsychobiology 13:31 (1985). Paroxetine is available commercially as a generic drug (Alphapharm; TorPharm; Sandoz), but also under the brand name PAXIL (Glaxo Smith Kline).

Paroxetine is an SSRI with indications for use for MDD, OCD, panic disorder, SAD, GAD, posttraumatic stress disorder (“PTSD”). Paroxetine is contraindicated for use with MAOI's. Paroxetine has only very weak effects on norepinephrine and dopamine neuronal reuptake. Paroxetine has little affinity for muscarinic, α₁-adrenergic, α₂-adrenergic, β-adrenergic, dopamine (D₂), 5-HT₁, 5-HT₂, and histamine (H₁) receptors. Paroxetine is completely absorbed after oral dosing with steady state concentrations achieved in approximately 10 days. Dosages typically range from 20-40 mg daily for the elderly and 20-50 mg daily for the nonelderly. Dosage ranges for paroxetine, can however, range from 10 mg/day to a maximum of 100 mg/day.

Paroxetine is extensively metabolized after oral administration. Approximately 64% of a 30 mg oral dose is renally excreted with 2% as the parent compound and 62% as metabolites over a 10 day period with the remaining 36% excreted in the feces. Initial dosages of paroxetine should be reduced in patients with renal or hepatic impairment.

Dosages of paroxetine for treatment of MDD, PTSD or GAD range from 20-50 mg/day. Dosages of paroxetine for treatment of OCD or SAD range from 20 to 60 mg/day. Dosages of paroxetine for treatment of panic disorder range from 10 to 60 mg/day. Dosages for the elderly range from 10 to not greater than 40 mg/day.

Particular embodiments described herein include paroxetine as the second component in combination with a mood stabilizer, such as lithium, in a single formulation. In other embodiments, paroxetine is administered in combination with lithium and a third component. The third component may be, but not limited to antiseizure drugs, atypical neuroleptics, and antipsychotic drugs as provided herein.

4. Sertraline

Sertraline, (1S-cis)-4-(3,4-dichlorophenyl)-1,2,3,4-tetrahydro-N-methyl-1-naphthylamine hydrochloride, is a serotonin reuptake inhibitor. It is disclosed by U.S. Pat. No. 4,536,518. Sertraline is commercially available as ZOLOFT (Pfizer).

Sertraline is an SSRI with weak effects on norepinephrine and dopamine neuronal uptake. Sertraline has no significant affinity for adrenergic (α₁, α₂, β), cholinergic, GABA, dopaminergic, histaminergic, serotonergic (5-HT_{1A}, 5-HT_{1B}, 5-HT₂), or benzodiazepine receptors. When sertraline is administered in a dose ranging from 50-200 mg for 14 days, mean peak plasma concentrations occur between 4.5 and 8.4 hours post dosing with steady state plasma levels achieved after approximately 1 week. Dosages, however, can start as low as 25 mg/day to as high as 250 mg/day. The average elimination half-life of sertraline is about 26 hours.

Sertraline is initially metabolized by N-demethylation, which causes sertraline to be less active. Sertraline is eliminated renally and fecally in approximately similar proportions. Elimination of sertraline in elderly patients is reduced approximately 40%.

Sertraline is indicated for use for MDD, OCD, PD, PTSD, PMDD and SAD and contraindicated for use with MAOI's. For MDD, OCD and PMDD, dosage is usually begins at 50 mg/day. PMDD dosages, however, can range from 50-150 mg/day. For a PD, PTSD and SAD indications, dosage ranges from 50-200 mg/day.

Particular embodiments described herein include sertraline as the second component in combination with a mood stabilizer, such as lithium, in a single formulation. In other embodiments, sertraline is administered in combination with lithium and a third component. The third component may be, but not limited to antiseizure drugs, atypical neuroleptics, and antipsychotic drugs as provided herein.

5. Fluoxetine

Fluoxetine, (±)-N-methyl-3-phenyl-[(α,α,α-trifluoro-p-tolyl)oxyl]propylamine hydrochloride, disclosed in U.S. Pat. Nos. 4,314,081 and 4,626,549. Fluoxetine pharmacology is reviewed in Beasley, et al., Psychopharmacology, 107:1-10 (1992) and Robertson, et al., J. Med. Chem. 31:1412-1417 (1988).

Fluoxetine is an SSRI, it is marketed as PROZAC and SARAFEM (Lilly). Following a single 40 mg dose, peak plasma concentrations is reached between 6 and 8 hours. Fluoxetine is a racemic mixture of R and S enantiomers, wherein both enantiomers are specific and potent, however, the S enantiomer is eliminated more slowly. The primary route of elimination is through hepatic metabolism to inactive metabolites that are excreted by the kidney.

Fluoxetine is eliminated relatively slowly, wherein the half life is 1 to 3 days following acute administration, and 4 to 6 days following chronic administration. After 30 days of dosing at 40 mg/day, plasma concentrations are in the range of 91-302 ng/ml.

Fluoxetine is indicated for use for MDD, OCD, bulimia nervosa, and panic disorder and contraindicated for use with MAOI's and thioridazine. In the average adult the dosage for treating MDD is 20 to 80 mg/day. For treatment for OCD, daily dosage of 20 to 60 mg/day is suggested. For bulimia nervosa, the indication is for 60 mg/day since lower dosages appear to have no significant difference compared with placebo. For treatment of panic disorder, the dose range is 10 to 60 mg/day.

Particular embodiments described herein include fluoxetine as the second component in combination with a mood stabilizer, such as lithium, in a single formulation. In other embodiments, fluoxetine is administered in combination with lithium and a third component. The third component may be, but not limited to antiseizure drugs, atypical neuroleptics, and antipsychotic drugs as provided herein.

6. Venlafaxine

Venlafaxine, (R/S)-1-[2-(dimethylamino)-1-(4-methoxyphenyl)ethyl] cyclohexanol hydrochloride or (±)-1-[α-[(dimethyl-amino)methyl]-p-methoxybenzyl] cyclohexanol hydrochloride, is known in the literature, and its method of synthesis and its activity as an inhibitor of serotonin and norepinephrine uptake are taught by U.S. Pat. No. 4,761,501.

Venlafaxine is an SSRI that is marketed as EFFEXOR and EFFEXOR XR (Wyeth) as tablets and as extended release capsules. Venlafaxine and its active metabolite, O-desmethylvenlafaxine (“ODV”) are potent inhibitors of neuronal serotonin and norepinephrine reuptake and weak inhibitors of dopamine reuptake. Venlafaxine and ODV have not significant affinity for muscarinic, histaminergic, or a-l adrenergic receptors in vitro.

Venlafaxine is well absorbed and extensively metabolized by the liver. With a single dose, 87% of the venlafaxine is renally excreted within 48 hours. Steady state concentrations of both venlafaxine and ODV in plasma are attained within 3 days of a multiple-dose therapy with dosages ranging from 75 to 450 mg/day. Venlafaxine has an elimination half-life of 3-7 hours and ODV has a half-life of 9-13 hours. For patients with renal or hepatic impairment it is recommended that dosages be decreased by 25-50%.

Venlafaxine is indicated for use for the treatment of MDD, generalized anxiety disorder (“GAD”), and SAD and is contraindicated for use with MAOI's. Dosages start at 75 mg/day, administered in two or three divided doses, taken with food. Dosage may be increased to a maximum of approximately 375 mg/day. Extended release venlafaxine can be administered once daily. For treatment of MDD, dosages typically start at 37.5 and can range to 375 mg/day for severely depressed patients. For GAD and SAD, dosages typically start at 75 mg/day, however, it may be desirable to start at 37.5 mg/day initially. Maximum dosages are approximately 225 mg/day for GAD and SAD.

When venlafaxine is administered at 150 mg/day with a single 600 mg oral dose of lithium to 12 healthy males, it was found that the steady-state pharmacokinetics of venlafaxine and ODV were unaffected. The steady-state pharmacokinetics of lithium was also unaffected by venlafaxine.

When venlafaxine is administered at 150 mg/day in 24 healthy subjects under steady state conditions, the clearance of a single 2 mg dose of haloperidol was found to decrease by 42% resulting in a 70% increase in the area under the plasma concentration curve. The peak plasma concentration (“Cₘₐₓ”) of haloperidol was also increased by 88% when co-administered with venlafaxine, however, the half life of haloperidol remain unchanged.

Particular embodiments described herein include venlafaxine as the second component in combination with a mood stabilizer, such as lithium, in a single formulation. In other embodiments, venlafaxine is administered in combination with lithium and a third component. The third component may be, but not limited to antiseizure drugs, atypical neuroleptics, and antipsychotic drugs as provided herein.

7. Duloxetine

Duloxetine hydrochloride, (+)-(S)-N-methyl-γ-(1-naphthyloxy)-2-thiophenepropylamine hydrochloride, is a selective serotonin and norepinephrine reuptake inhibitor (SSNRI) and is described in U.S. Pat. Nos. 5,023,269 and 4,956,388 by Robertson, et al., and the synthesis of duloxetine is discussed by Deeter, et al., Tetrahedron Letters, 31 (49), 7101-04 (1990). Duloxetine hydrochloride is commercially available as CYMBALTA (Eli Lilly) in 22.4, 33.7 or 67.3 mg enteric coated tablet dosages which is equivalent to 20, 30 and 60 mg of duloxetine, respectively.

Duloxetine has no significant affinity for dopaminergic, adrenergic, cholinergic, or histaminergic receptors in vitro. Duloxetine does not inhibit MAO. Duloxetine is extensively metabolized hepatically, however, metabolites do not appear to contribute significantly to its pharmacologic activity. It is recommended that patients with hepatic insufficiency not be administered duloxetine. Duloxetine has an elimination half-life of about 8 to 17 hours and its pharmacokinetics are dose proportional over the therapeutic range. Steady-state plasma concentrations are normally reached after 3 days of dosing.

Duloxetine is indicated for use for major depressive disorder and is contraindicated for patients with known hypersensitivity, for concomitant use with MAOIs, and for patients with uncontrolled narrow-angle glaucoma. Typical dosage of duloxetine is a total does of 40 mg/day to 60 mg/day without regard to meals.

Particular embodiments described herein include duloxetine as the second component in combination with a mood stabilizer, such as lithium, in a single formulation. In other embodiments, duloxetine is administered in combination with lithium and a third component. The third component may be, but not limited to antiseizure drugs, atypical neuroleptics, and antipsychotic drugs as provided herein.

### C. Antiseizure Drugs

1. Oxcarbazepine

Oxcarbazepine, 10,11-dihydro-10-oxo-5H-dibenzo[b,f]azepine-5-carboxamide is known and its preparation and the therapeutic use as anticonvulsive drug is described in German Auslegeschrift 20 11 087 and Patent EP 50,589. An industrially useful process for the preparation of this drug is described in European patent specification number 028 028. Commercially available dosage forms are provided for peroral administration in tablets containing 150, 300 and 600 mg of active drug. These dosage forms are commercially sold under the brand name TRILEPTAL, (Novartis).

Oral administration of oxcarbazepine is completely absorbed and extensively metabolized to its pharmacologically active metabolite 10-monohydroxy metabolite (“MHD”). The half life of oxcarbazepine is about 2 hours, and the half life of MHD is about 9 hours. Steady state plasma concentrations of MHD are reached within 2-3 days in patients given oxcarbazepine twice daily. At steady-state, the pharmacokinetics of MHD are linear and show dose proportionality over the dose range of 300-2400 mg/day. Oxcarbazepine is hepatically metabolized and renally excreted.

Oxcarbazepine is indicated for use as a monotherapy or adjunctive therapy in the treatment of partial seizures in adults and children with epilepsy. Treatment with oxcarbazepine should be initiated with a dose of 600 mg/day, given in a BID regimen. The dose may be increased by a maximum of 600 mg/day at approximately weekly intervals with the recommended daily dose of 1200 mg/day. Patients were found to not be able to tolerate a 2400 mg/day dose due to CNS effects. Patients with renal impairment should have dosages halved and increased slowly until desired clinical response.

Particular embodiments described herein include oxcarbazepine as the third component in combination with lithium and an SSRI. In other embodiments, oxcarbazepine is administered in combination with lithium and an SSRI in a single formulation. In another embodiment, oxcarbazepine is administered in combination with lithium in a single formulation.

2. Lamotrigine

Lamotrigine, 3,5-diamino-6-(2,3-dichlorophenyl)-1,2,4-triazine and is disclosed in European patents 21121 and 247829; and U.S. Pat. No. 4,602,017. Lamotrigine is typically used in the treatment of status epilepticus (“SE”) and is commercially available under the trade name LAMICTAL as an oral tablet formulation. Typically, a therapeutic dose of lamotrigine for an adult human being comprises a loading dose of, approximately 1000-1500 mg followed by a maintenance dose of, approximately 500-700 mg/day. Plasma concentrations of lamotrigine of about 24 μg/ml are generally effective in controlling SE for add-on therapy, such as when taken concomitantly with other anti-epileptic drugs (“AEDs”). Plasma concentrations of about 8-10 g1g/ml are generally effective for monotherapy.

Lamotrigine is indicated for use for Epilepsy as well as for maintenance treatment of Bipolar I Disorder to delay the time to occurrence of mood episodes (such as, depression, mania, hypomania, mixed episodes). Lamotrigine is contraindicated in patients with a demonstrated hypersensitivity. For treatment of Bipolar disorder, it was found that a dose of approximately 200 mg/day, ranging from 100 mg/day to 400 mg/day, was effective in delaying the time to occurrence of a mood episode.

Particular embodiments described herein include lamotrigine as the third component in combination with lithium and an SSRI. In other embodiments, lamotrigine is administered in combination with lithium and an SSRI in a single formulation. In another embodiment, lamotrigine is administered in combination with lithium in a single formulation. In yet another embodiment lamotrigine is administered in combination with bupropion in a single formulation. In yet a further embodiment, lamotrigine is administered with li+bupropion in a single form.

3. Topiramate

Topiramate, 2,3:4,5-Di-O-isopropylidene-β-D-fructopyranose sulfamate is known and its preparation and the therapeutic use as anticonvulsive drug is described in U.S. Pat. No. 4,513,006. Topiramate is commercially available as TOPOMAX (Ortho McNeil) in 25, 50, 100, 200, 300, and 400 mg tablets and as TOPOMAX Sprinkle in 15 and 25 mg capsules.

Absorption of topiramate is rapid, with peak plasma concentrations occurring at approximately 2 hours following a 400 mg oral dose. The mean plasma elimination half-life is 21 hours after single or multiple doses. Steady state is reached in about 4 days with normal renal function. Topiramate is not extensively metabolized and is primarily eliminated unchanged in urine. Clearance was reduced by 42% in moderately renally impaired patients and 54% in severely in renally impaired subjects compared to normal.

Topiramate is indicated for use as adjunctive therapy for adults and pediatric patients ages 2-16 with partial onset seizures, or primary generalized tonic-clonic seizures, and in patients 2 years of age and older with seizures associated with Lennox-Gastaut syndrome. Topiramate is contraindicated in patients with hypersensitivity to it. Dosages for adults is 200-400 mg/day in two divided doses as adjunctive therapy in adults with partial seizures. Dosages for adults with primary generalized tonic-clonic seizures is 400 mg/day in two divided dosages.

It is contemplated that particular embodiments include topiramate as the third component in combination with a mood stabilizer and an SSRI. In other embodiments, topiramate is administered in combination with lithium and an SSRI in a single formulation. In another embodiment topiramate is administered in combination with lithium in a single formulation.

4. Divalproex Sodium

Divalproex sodium is a stable co-ordination compound comprised of sodium valproate and valproic acid in a 1:1 molar relationship and formed during the partial neutralization of valproic acid with 0.5 equivalent of sodium hydroxide. Chemically it is sodium hydrogen bis(2-propylpentanoate). Divalproex sodium is known and its preparation and therapeutic use as an anticonvulsive drug is described in U.S. Pat. Nos. 4,988,731 and 5,212,326. Divalproex sodium is available commercially as DEPAKOTE and DEPAKOTE ER (Abbott) in 125, 250 and 500 mg dosages.

Divalproex sodium is indicated for use for migraine headaches in adults, monotherapy and adjunctive therapy in the treatment of adult patients with complex partial seizures. Divalproex sodium is also indicated for use as a sole and adjunctive therapy in the treatment of single and complex absence seizures in adult patients and adjunctively in adult patients with multiple seizure types that include absence seizures. Divalproex sodium is contraindicated for patients with hepatic disease or significant hepatic dysfunction. When valproate (500 mg BID) is co-administered with lithium carbonate (300 mg TID) to normal male volunteers, there was not effect on the steady state kinetics of lithium. Dosage ranges for divalproex can be as high as 2400 mg/day.

It is contemplated that particular embodiments include divalproex sodium as the third component in combination with lithium and an SSRI. In other embodiments, divalproex sodium is administered in combination with lithium and an SSRI in a single formulation. In another embodiment divalproex sodium is administered in combination with lithium in a single formulation. A further embodiment includes divalproex sodium as the second component in combination with bupropion.

5. Levetiracetam

Levetiracetam, (−)-(S)-α-ethyl-2-oxo-1-pyrrolidine acetamide is known and its preparation is described in U.S. Pat. Nos. 4,943,639 and 4,837,223. It is commercially available as KEPPRA (UCB Pharma) in 250, 500, and 750 mg tablets.

Levetiracetam is rapidly and almost completely absorbed after oral administrations with peak plasma concentrations occurring in about an hour. Levetiracetam has a plasma half-life in adults that is approximately 7 hours that is unaffected by either dose or repeated administration. Daily dosage ranges can be as high as 2400 mg/day. Levetiracetam is renally excreted, and is reduced in patients with renal impairment (decreased 50% with moderate renal impairment, 60% for severe renal impairment). Levetiracetam is indicated for use as an adjunctive therapy in the treatment of partial onset seizures in adults with epilepsy.

It is contemplated that particular embodiments include levetiracetam as the third component in combination with lithium and an SSRI. In other embodiments, levetiracetam is administered in combination with lithium and an SSRI in a single formulation. In another embodiment levetiracetam is administered in combination with lithium in a single formulation.

6. Gabapentin

Gabapentin, 1-(aminoethyl)cyclohexaneacetic acid is known and methods for its preparation are described in U.S. Pat. Nos. 4,894,476; 6,054,482; 4,024,175 and 4,087,544. Gabapentin is commercially available as NEURONTIN (Pfizer, New York) as an 100 mg, 300 mg, 400 mg, 600 mg, and 800 mg tablets or a 250 mg/5 ml oral solution.

Gabapentin is structurally related to the neurotransmitter GABA (γ-aminobutyric acid) but it does not modify GABA_{A} or GABA_{B} radioligand binding, it is not converted metabolically into GABA or a GABA agonist, and it is not an inhibitor of GABA uptake or degradation. At concentrations of 100 μM, gabapentin does not exhibit affinity for receptors for benzodiazepine, glutamate, N-methyl-D-aspartate (NMDA), quisqualate, kainite, strychnine-insensitive or strychnine-sensitive glycine, α1, α2, or β-adrenergic, adenosine A1 or A2, cholinergic muscarinic or nicotinic, dopamine D1 or D2, histamine H1, serotonin S1 or S2, opiate μ, δ, κ, canabinoid 1, voltage-sensitive calcium channel sites labeled with nitrendipine or diltiazem, or at voltage-sensitive sodium channel sites labeled with batrachotoxinin A 20-α-benzoate. Gabapentin also does not alter the cellular uptake of dopamine, noradrenaline, or serotonin.

Humans do not appreciably metabolize gabapentin, its effect is due to the activity of the parent compound. Gabapentin bioavailability is not dose proportional, as dose increases, bioavailability decreases. Bioavailability of gabapentin is approximately 60%, 47%, 34%, 33% and 27% following 900, 1200, 2400, 3600, and 4800 mg/day given in 3 divided doses, respectively. Gabapentin is eliminated renally as unchanged drug and the elimination rate is directly proportional to creatinine clearance. The elimination half-life is 5 to 7 hours and is unaltered by dose or following multiple dosing. Dosages need to be modified for the renally impaired. Gabapentin is given orally with or without food. Dosages range from 900 mg/day to 1800 mg/day. Gabapentin is indicated for use for postherpetic neuralgia and epilepsy.

It is contemplated that particular embodiments include gabapentin as the third component in combination with lithium and an SSRI. In other embodiments, gabapentin is administered in combination with lithium and an SSRI in a single formulation. In another embodiment gabapentin is administered in combination with lithium in a single formulation.

7. Tiagabine

Tiagabine, (−)-®-1-[4,4-Bis(3-methyl-2-thienyl)-3-butenyl]nipecotic acid hydrochloride, is known and methods for its preparation are described in U.S. Pat. Nos. 5,010,090, 5,354,760, and Danish Patent 156398. Tiagabine is commercially available as GABITRIL (Cephalon, Pennsylvania) in 2, 4, 12, 16, and 20 mg tablets.

Tiagabine appears to enhance the activity of GABA by binding recognition sites associated with the GABA uptake carrier. It is postulated that tiagabine blocks GABA uptake into presynaptic neurons, permitting more GABA to be available for receptor binding on surfaces of post-synaptic cells. Tiagabine does not significantly inhibit the uptake of dopamine, norepinephrine, serotonin, glutamate, or choline and with little or no binding to dopamine D1 and D2, muscarinic, serotonin 5HT_{1A}, 5HT₂, 5HT₃, β-1 and 2 adrenergic, α-1 and α-2 adrenergic, histamine H2 and H3, adenosine A₁ and A₂, opiate μ and κ₁, NMDA glutamate and GABA_{A} receptors, or to sodium or calcium channels. Tiagabine binds to histamine H1, serotonin 5HT_{1B}, benzodiazepine, and chloride channel receptors at concentrations greatly exceeding that is needed to inhibit the uptake of GABA.

Tiagabine has an elimination half-life of 7 to 9 hours and a half-life of 4 to 7 hours in patients receiving hepatic enzyme-inducing drugs. Absorption of tiagabine is rapid, with peak plasma concentrations occurring at approximately 45 minutes following an oral dose in fasting state. Tiagabine appears to be hepatically metabolized with 2% excreted unchanged, 25% excreted renally and 63% excreted in the feces as metabolites.

Tiagabine is indicated for use for treatment of partial seizures. Dosages for adults range from 4 mg to 56 mg/day. Doses are initiated at 4 mg/day and increased by 4-8 mg weekly until therapeutic effect is seen.

It is contemplated that particular embodiments include tiagabine as the third component in combination with lithium and an SSRI. In other embodiments, tiagabine is administered in combination with lithium and an SSRI in a single formulation. In another embodiment tiagabine is administered in combination with lithium in a single formulation.

8. Zonisamide

Zonisamide, 1,2-benzisoxazole-3-methanesulfonamide, is known and methods for its preparation are described in Japanese Pat. No. 53-77057 and Yakugaku Zasshi, 6 (7), 533-47, 1996; U.S. Pat. No. 4,172,896 and Japanese Patent. No. 53-77057 to Dainnipon. Zonisamide is commercially available as ZONEGRAN (Elan Biopharmaceuticals, California) in 25, 50, or 100 mg capsules.

Following a 200-400 mg oral dose, peak plasma concentrations in normal volunteers occur within 2-6 hours. Zonisamide binds extensively to red blood cells. The pharmacokinetics of zonisamide is dose proportional in the range of 200-400 mg, but the Cₘₐₓ and AUC increase disproportionately at 800 mg. Steady state is achieved within 14 days. The elimination half-life of zonisamide in plasma is about 63 hours. The elimination half-life of zonisamide in RBC is approximately 105 hours. Zonisamide is hepatically metabolized and primarily excreted renally.

Zonisamide is indicated for adjunctive therapy for the treatment of partial seizures in adults. Dosages range from 100 to 600 mg/day. The initial dose should be 100 mg/day, after two weeks, the dose may be increased by another 100 mg/day for another two weeks. Dosages should be titrated on a biweekly bases. Dosages should be adjusted for the hepatically and/or renally impaired.

It is contemplated that particular embodiments include zonisamide as the third component in combination with lithium and an SSRI. In other embodiments, zonisamide is administered in combination with lithium and an SSRI in a single formulation. In another embodiment zonisamide is administered in combination with lithium in a single formulation.

### D. Atypical Neuroleptics

1. Aripiprazole

Aripiprazole, 7-[4-[4-(2,3-dichlorophenyl)-1-piperazinyl]butoxy]-3,4-dihydrocarbostyril is known and methods for its preparation are described in U.S. Pat. No. 5,006,528. Aripiprazole is commercially available as ABILIFY (Otsuka) in 5, 10, 15, 20, and 30 mg tablets.

Aripiprazole exhibits high affinity for dopamine D₂ and D₃, serotonin 5-HT_{1A} and 5-HT_{2A} receptors, moderate affinity for dopamine D₄, serotonin 5-HT_{2C} and 5-HT₇, α-adrenergic and histamine H1 receptors, and moderate affinity for the serotonin reuptake site. Aripiprazole has no appreciable affinity for cholinergic muscarinic receptors. Aripiprazole functions as a partial agonist at the dopamine D₂ and the serotonin 5-HT_{1A} receptors, and as an antagonist at serotonin 5-HT_{2A} receptor.

Aripiprazole is well absorbed, with peak plasma concentrations occurring within 3 to 5 hours. No dosage adjustments appear to be necessary for hepatic and renal impairment. Co-administration with lithium (1200-1800 mg/day) for 21 days with aripiprazole (30 mg/day) did not result in clinically significant changes in the pharmacokinetics of aripiprazole or its active metabolite, dehydro-aripiprazole.

Aripiprazole is indicated for use for schizophrenia. The recommended starting dose and target dose for aripiprazole is 10 or 15 mg/day administered once a day with effective dose range to be 10-30 mg/day.

It is contemplated that particular embodiments include aripiprazole as the third component in combination with lithium and an SSRI. In other embodiments, aripiprazole is administered in combination with lithium and an SSRI in a single formulation.

2. Quetiapine Fumarate

Quetiapine fumarate, 5-[2-(4-dibenzo[b,f][1,4]thiazepin-11-yl-1-piperazinyl)ethoxy]ethanol fumarate, and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt. Quetiapine fumarate is available commercially as SEROQUEL (Astrazeneca) in 25, 100, 200, and 300 mg tablets.

Quetiapine fumarate is an antagonist for serotonin 5-HT_{1A} and 5-HT₂ receptors, dopamine D₁ and D₂ receptors, histamine H1 receptors, and adrenergic α₁ and α₂ receptors. Quetiapine fumarate has no appreciable affinity for cholinergic, muscarinic, and benzodiazepine receptors.

Elimination of quetiapine is mainly hepatic with a mean terminal half-life of about 6 hours. Steady-state concentrations are expected to be achieved within two days of dosing. Peak plasma concentrations is reached in 1.5 hours after oral administration.

Quetiapine fumarate is indicated for use for short-term treatment of acute manic episodes associated with bipolar I disorder, as either monotherapy or adjunct to lithium or divalproex. Quetiapine fumarate is also indicated for use for treatment of schizophrenia.

Coadministration of quetiapine fumarate (150 mg BID) and divalproex (500 mg BID) increased the mean maximum plasma concentration of quetiapine at steady state by 17% without affecting the extent of absorption or mean oral clearance. Coadministration of fluoxetine (60 mg once daily); haloperidol (7.5 mg BID, or risperidone (3 mg BID) with quetiapine fumarate (300 mg BID) did not alter the steady-state pharmacokinetics of quetiapine. Coadministration of quetiapine (250 mg TID) with lithium had no effect on any effect on any of the steady-state pharmacokinetic parameters of lithium.

Dosages for use as monotherapy or adjunct therapy (with lithium or divalproex) for bipolar mania, should be initiated in BID doses totaling 100 mg/day on day 1, increased to 400 mg/day on Day 4 in increments of up to 100 mg/day in BID divided doses. Further dosage adjustments up to 800 mg/day by Day 6 should be in increments no greater than 200 mg/day.

It is contemplated that particular embodiments include quetiapine fumarate as the third component in combination with lithium and an SSRI. In other embodiments, quetiapine fumarate is administered in combination with lithium and an SSRI in a single formulation. It is also contemplated that quetiapine fumarate is administered with lithium in a single formulation. It is also contemplated that quetiapine fumarate is administered with an antiseizure medication, such as, but not limited to, divalproex sodium, lamotrigine, oxcarbazepine, topiramate, or levetiracetam.

3. Risperidone

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663, which is herein incorporated by reference in its entirety. Risperidone is commercially available as RISPERDAL (Janssen) in 0.25, 0.5, 1, 2, 3, and 4 mg tablets and as a long acting intramuscular preparation commercially known as CONSTA (Janssen).

Risperidone is a selective monoaminergic antagonist with high affinity for the serotonin 5-HT₂, dopamine D₂, α₁ and α₂ adrenergic, and H₁ histaminergic receptors. Risperidone acts as an antagonist at other receptors, but with lower potency. Risperidone has low to moderate affinity for the serotonin 5-HT_{1C}, 5-HT_{1D} and 5-HT_{1A} receptors, weak affinity for the dopamine D₁ and haloperidol-sensitive sigma site, and no affinity for cholinergic muscarinic or β₁ and β₂ adrenergic receptors.

Plasma concentrations of risperidone, its major metabolite, 9-hydroxyrisperidone, and risperidone plus 9-hydroxyrisperidone are dose proportional over the dosing range of 1 to 16 mg daily. Peak plasma concentrations of risperidone occurred at about 1 hour. Peak concentrations of 9-hydroxyrisperidone occurred at about 3 hours in extensive metabolizers, and 17 hour in poor metabolizers. Steady state concentrations of risperidone are reached in 1 day in extensive metabolizers and expected to reach steady state in about 5 days in poor metabolizers. Steady state concentrations of 9-hydroxyrisperidone are reached in 5-6 days.

Risperidone is extensively metabolized in the liver by CYP 2D6. Risperidone and its metabolites are primarily eliminated renally. The half-life of risperidone was 3 hours in extensive metabolizers and 20 hours in poor metabolizers. The approximate half-life of 9-hydroxyrisperidone is about 21 hours in extensive metabolizers and 30 hours in poor metabolizers. Clearance of risperidone and its metabolites were decreased by 60% for the renally impaired. Those with hepatic impairment had a 35% increase in the mean free fraction of risperidone in plasma.

Risperidone is indicated for use for the treatment of schizophrenia and bipolar mania. The combination of risperidone with lithium or valproate (divalproex sodium) is indicated for short-term treatment of acute manic or mixed episodes associated with Bipolar I disorder. When risperidone is co-administered with fluoxetine (20 mg QD), plasma concentrations of risperidone is increased 2.5-2.8 fold, while the concentration of 9-hydroxyrisperidone was not affected. When risperidone is co-administered with lithium, the AUC and the peak plasma concentrations of lithium were unaffected. Efficacy in schizophrenia was demonstrated in a dose range of 4-16 mg/day. For treatment of Bipolar Mania the usual dose ranged from 1-6 mg/day.

It is contemplated that particular embodiments include risperidone as the third component in combination with lithium and an SSRI. In other embodiments, risperidone is administered in combination with lithium and an SSRI in a single formulation. It is also contemplated that risperidone is administered with lithium in a single formulation.

4. Ziprasidone

Ziprasidone, 5-[2-[4-(1,2-benzoisothiazol-3-yl)-1-piperazinyl]ethyl]-6-chloro-1,3-dihydro-2H-indol-2-one, is typically administered as the hydrochloride monohydrate. The compound is described in U.S. Pat. Nos. 4,831,031 and 5,312,925. Its activity in assays which demonstrate utility in the treatment of schizophrenia are described in U.S. Pat. No. 4,831,031. U.S. Pat. Nos. 4,831,031 and 5,312,925 are herein incorporated by reference in their entirety. Ziprasidone is commercially available as GEODON (Pfizer) in 20, 40, 60, and 80 mg capsules.

Ziprasidone exhibited high in vitro binding affinity for dopamine D₂ and D₃, serotonin 5-HT_{2A}, 5-HT_{2C}, 5-HT_{1D} and α-adrenergic receptors and moderate affinity for the histamine H₁ receptor. Ziprasidone functioned as an antagonist at the D₂, 5-HT_{2A}, and 5-HT_{1D} receptors, and as an agonist at the 5-HT_{1A} receptor. Ziprasidone also inhibited synaptic reuptake of serotonin and norepinephrine. No appreciable affinity was exhibited for other receptor/binding sites tested, including the cholinergic muscarinic receptor.

Ziprasidone's activity is primarily due to the parent drug. Multiple-dose pharmacokinetics of ziprasidone are dose-proportional within the clinical dose range. Ziprasidone is eliminated via hepatic metabolism with a mean terminal half-life of about 7 hours. Steady state concentrations are achieved within one to three days of dosing.

Ziprasidone is indicated for use for the treatment of schizophrenia. When ziprasidone (40 mg BID) is co-administered with lithium (450 mg BID for 7 days), the steady state level and renal clearance of lithium was not affected. Dosages typically range from 20-100 mg BID for short-term treatment of schizophrenia. Dose ranges can be adjusted on the basis of individual clinical status and can be as high as 240 mg/day.

It is contemplated that particular embodiments include ziprasidone as the third component in combination with lithium and an SSRI. In other embodiments, ziprasidone is administered in combination with lithium and an SSRI in a single formulation. It is also contemplated that ziprasidone is administered with lithium in a single formulation.

5. Olanzapine

Olanzapine, 2-methyl-4-(4-methyl-1-piperazinyl)-10H-thieno[2,3-b][1,5]benzodiazepine, is a known compound and is described in U.S. Pat. No. 5,229,382 as being useful for the treatment of schizophrenia, schizophreniform disorder, acute mania, mild anxiety states, and psychosis. U.S. Pat. No. 5,229,382 is herein incorporated by reference in its entirety. Olanzapine is commercially available as ZYPREXA and ZYPREXA ZYDIS (orally disintegrating tablets) (Lilly). Olanzapine is available in 2.5, 5, 7.5, 10, 15, and 20 mg tablets. The orally disintegrating form comes in 5, 10, 15, and 20 mg tablets.

Olanzapine is a selective monoaminergic antagonist with high affinity binding to serotonin 5-HT_{2A/2C}, dopamine D₁₋₄, muscarinic M₁₋₅, histamine H₁, and adrenergic α₁ receptors. Olanzapine binds weakly to GABAA, BZD, and β adrenergic receptors.

Olanzapine reaches peak concentrations in approximately 6 hours following an oral dose. It is eliminated by first pass metabolism, with approximately 40% of the dose metabolized before reaching system circulation. Olanzapine's half-life ranges from 21 to 54 hours. Once daily administration leads to steady-state concentrations in about 1 week.

Olanzapine is indicated for the treatment of schizophrenia as well as bipolar disorder. With regard to acute monotherapy of Bipolar Disorder, olanzapine is indicated for treatment of acute mixed or manic episodes. Olanzapine can also be used for maintenance monotherapy for bipolar disorder. Olanzapine may also be combined with lithium or valproate (divalproex sodium) for short-term treatment of acute manic episodes associated with Bipolar I Disorder.

When olanzapine is co-administered with a 60 mg single dose or 60 mg daily dose for 8 days of fluoxetine, there is a small increase in the maximum concentration of olanzapine and a small decrease in its clearance. When olanzapine is co-administered with fluvoxamine, the clearance of olanzapine decreases. There is a mean increase in olanzapine Cmax following fluvoxamine of 54% in female non smokers and 77% in male smokers with a mean increase of 52% and 108%, respectively, of the AUC. Multiple doses of olanzapine (10 mg for 8 days) did not influence the kinetics of lithium. When olanzapine is co-administered with valproate, little effect on each drug's metabolism was seen.

The dosage range of olanzapine for the treatment schizophrenia is 10-20 mg/day. The dosage range for bipolar disorder is 5-20 mg/day. Dosage range in combination therapy with lithium or valproate ranges from 5-20 mg/day.

It is contemplated that particular embodiments include olanzapine as the third component in combination with lithium and an SSRI. In other embodiments, olanzapine is administered in combination with lithium and an SSRI in a single formulation. It is also contemplated that olanzapine is administered with lithium or divalproex sodium in a single formulation.

### E. Antipsychotic Drugs

1. Haloperidol

Haloperidol, 4-[4-(p-chlorophenyl)-4-hydroxypiperidino]-4′-fluorobutyrophenone is known and described in Janssen, Int. Rev. Neurobiol., 8:221-263 (1965) and Janssen, In Psychopharmcological Agents, Vol 3 (Gordon, M. ed.) Academic Press, Inc., New York, 1974, pp. 128-158. Haloperidol is available commercially as a generic drug as well as under the brand name HALDOL (Ortho McNeil) in many different formulations including but not limited to tablets, oral concentrates, and intramuscular (“i.m.”) injection.

Haloperidol is indicated for use in the management of manifestations of acute and chronic psychotic disorders including schizophrenia and manic states. It has been considered particularly effective in the management of hyperactivity, agitation, mania, and controlling tics and vocal utterances of Tourette's Disorder.

Peak plasma levels of haloperidol occur with 2 to 6 hours of oral dosing and about 20 minutes after i.m. injection. The mean plasma half-life is approximately 15 to 25 hours. Dosages should be individualized and vary widely depending on symptoms, age, weight, and health. Dosages range from 0.5 mg to 100 mg/day.

It is contemplated that particular embodiments include haloperidol as the third component in combination with lithium and an SSRI. In other embodiments, haloperidol is administered in combination with lithium and an SSRI in a single formulation. It is also contemplated that haloperidol is administered with lithium in a single formulation.

2. Fluphenazine

Fluphenazine, 4-[3-[2-trifluoromethyl)phenothiazin-10-yl]propyl]-1-piperazineethanol is known and its pharmacology is described by High, el al., Toxicol. Appl. Pharmacol., 2:540-52 (1960). Fluphenazine is commercially available as a hydrochloride, enanthate, and a decanoate under the brand names PROLIXIN (Apothecon), PERMITIL (Schering), and MODECATE (various suppliers).

Fluphenazine is indicated for use in the management of manifestations of psychotic disorders, such as schizophrenia. It is contraindicated in patients with suspected or established subcortical brain damage, in patients receiving large doses of hypnotics and in comatose or severely depressed states. Dosages range from 2.5 mg to 100 mg per day.

It is contemplated that particular embodiments include fluphenazine as the third component in combination with lithium and an SSRI. In other embodiments, fluphenazine is administered in combination with lithium and an SSRI in a single formulation. It is also contemplated that fluphenazine is administered with lithium in a single formulation.

3. Chlorpromazine Hydrochloride

Chlorpromazine hydrochloride's chemical name is 10-(3-dimethylaminopropyl)-2-chlorphenothiazine. It is commercially available as THORAZINE (GlaxoSmithKline) in tablets (10, 25, 50, 100, 200 mg), capsules (30, 75, 150 mg), ampoules (1, 2mL), multi-dose vials (10 mL), syrup (10 mg/5 mL), and suppositories (25, 100 mg).

Chlorpromazine hydrochloride is indicated for treatment of schizophrenia, to control nausea and vomiting, for relief of restlessness and apprehension before surgery, for acute intermittent porphyria, as an adjunct in the treatment of tetanus, to control the manifestations of the manic type of manic-depressive illness, for relief of intractable hiccups, for the treatment of severe behavioral problems in some children, and for the short-term treatment of some hyperactive children. Chlorpromazine hydrochloride is contraindicated for use in patients with known hypersensitivity to phenothiazines, nor should it be used in comatose states or in the presence of large amounts of central nervous system depressants (alcohol, barbiturates, narcotics, etc.).

Dosages must be adjusted to the individual and the severity of the condition, increasing it until symptoms are controlled. In continued therapy, dosages should be reduced to the lowest effective maintenance level, after symptoms have been controlled for a reasonable period. The 100 mg and 200 mg tablets are for use in severe neuropsychiatric conditions. For treatment of psychotic disorders, daily dosage of 200 mg is not unusual. Some patients may require higher doses (for example, but not limited to, 800 mg daily is not uncommon in discharged mental patients).

Chlorpromazine hydrochloride is readily absorbed from the gastrointestinal tract; however its bioavailability is variable due to considerable first pass metabolism by the liver. The terminal half-life of chlorpromazine is variable at approximately 30 hours.

It is contemplated that particular embodiments include chlorpromazine hydrochloride as the second component in combination with lithium. It is further contemplated that chlorpromazine hydrochloride is administered with lithium in a single formulation.

4. Thioridazine Hydrochloride

Thioridazine hydrochloride's chemical name is 2-methylmercapto-10-[2-(N-methyl-2-piperidyl) ethyl]phenothiazine. Thioridazine hydrochloride is commercially available as MELLARIL (Novartis) in the form of tablets (10, 15, 25, 50, 100, 150, and 200 mg) and oral solution (30, 100 mg/mL). MELLARIL-S, or thioridazine, is available as an oral suspension (5, 20 mg/mL).

Thioridazine hydrochloride is indicated for the management of schizophrenic patients who fail to respond adequately to treatment with other antipsychotic drugs because of insufficient effectiveness or the inability to achieve an effective dose due to intolerable adverse effects from those drugs. Thioridazine hydrochloride use should be avoided in combination with other drugs that are known to prolong the QTc interval and in patients with congenital long QT syndrome or a history of cardiac arrhythmias. It is also contraindicated for use in patients with reduced cytochrome P450 2D6 isozyme activity, with drugs that inhibit this isozyme (non-limiting examples include, fluoxetine and paroxetine), and with certain other drugs (non-limiting examples include, fluvoxamine, propranolol, and pindolol) that appear to appreciably inhibit the metabolism of thioridazine. Thioridazine hydrochloride is also contraindicated for severe central nervous system depression or comatose states from any cause including drug induced central nervous system depression.

The total daily dosage for adult schizophrenic patients ranges from 200-800 mg, divided into two to four doses. Thioridazine is rapidly and completely absorbed from the gastrointestinal tract. Maximum plasma concentrations are reached 2 to 4 hours after ingestion. Thioridazine is metabolized in the liver. Excretion is mainly via the feces (50%), but also via the kidney (less than 4% as unchanged drug, about 30% as metabolites). Plasma elimination half-life is approximately 10 hours.

It is contemplated that particular embodiments include thioridazine hydrochloride as the second component in combination with lithium in a single formulation.

5. Trifluoperazine Hydrochloride

Trifluoperazine hydrochloride's chemical name is 10-[3-(4-methylpiperazin-1-yl)propyl]2-(trifluoromethyl)-10H-phenothiazine. It is commercially available as STELAZINE (GlaxoSmithKline) in 1, 2, 5, 10 mg tablets, 10 mL multi-dose vials, and concentrate forms. Trifluoperazine hydrochloride is indicated for management of schizophrenia and is effective for the short-term treatment of generalized non-psychotic anxiety. It is contraindicated for known hypersensitivity to phenothiazines, comatose or greatly depressed states due to central nervous system depressants and, in cases of existing blood dyscrasias, bone marrow depression and pre-existing liver damage.

Usual dosage for treating non-psychotic anxiety is 1 or 2 mg twice daily. Most patients requiring treatment for psychotic disorders will show optimum response on 15 mg or 20 mg orally daily, although a few may require 40 mg a day or more.

It is contemplated that particular embodiments include trifluoperazine hydrochloride as the second component in combination with lithium. It is further contemplated that trifluoperazine hydrochloride is administered with lithium in a single formulation.

6. Thiothixene Hydrochloride

Thiothixene hydrochloride is the cis isomer of N,N-dimethyl-9-[3-(4-methyl-1-piperazinyl)-propylidene]thioxanthene-2-sulfonamide. It is available in commercial form as NAVANE (Pfizer) in 1, 2, 5, 10, and 20 mg capsule form.

Thiothixene hydrochloride is indicated in the management of schizophrenia. It is contraindicated in patients with circulatory collapse, comatose states, central nervous system depression due to any cause, and blood dyscrasias. Thiothixene hydrochloride is also contraindicated in patients with shown hypersensitivity to the drug.

The usual optimal dose is 20 to 30 mg daily. If indicated, an increase to 60 mg/day total daily dose is often effective.

It is contemplated that particular embodiments include thiothixene hydrochloride as the second component in combination with lithium. It is further contemplated that thiothixene hydrochloride is administered with lithium in a single formulation.

7. Loxapine Succinate

Loxapine succinate's chemical name is 2-Chloro-11-(4-methyl-1-piperazinyl)dibenz[b,f][1,4]oxazepine. It is commercially available as LOXITANE (Watson) in 5, 10, 25, or 50 mg capsules for oral administration.

Loxapine succinate is administered orally, usually in divided doses 2 to 4 times a day. Initial dosage of 10 mg twice daily is recommended, although in severely disturbed patients initial dosage up to a total of 50 mg daily may be desirable. The usual therapeutic range is 60 to 100 mg daily, while daily dosages higher than 250 mg are not recommended. Many patients have been maintained satisfactorily at dosages in the range of 20 to 60 mg daily.

Absorption following orally administered loxapine succinate is virtually complete. It is metabolized extensively and is excreted mainly in the first 24 hours. Metabolites are excreted in the urine in the form of conjugates and in the feces unconjugated.

It is contemplated that particular embodiments include loxapine succinate as the second component in combination with lithium. It is further contemplated that loxapine succinate is administered with lithium in a single formulation.

8. Perphenazine

Perphenazine's chemical name is 4-[3-(2-chlorophenothiazin-10-yl)propyl]-1-piper-azineethanol. It is commercially available as TRILAFON (Shering) as 2, 4, 8, and 16 mg tablet and 5 mg injection forms.

Perphenazine is indicated for use in the treatment of schizophrenia and for the control of severe nausea and vomiting in adults. It is contraindicated in comatose or greatly obtunded patients, in patients receiving large doses of central nervous system depressants, and in the presence of existing blood dyscrasias, bone marrow, depression, or liver damage. Perphenazine is also contraindicated in patients who have shown hypersensitivity to perphenazine and in patients with suspected or established subcortical brain damage.

Prolonged administration of doses exceeding 24 mg daily should be reserved for hospitalized patients or patients under continued observation for early detection and management of adverse reactions. Following oral administration of perphenazine tablets, mean peak plasma perphenazine concentrations were observed between 1 to 3 hours. The plasma elimination half-life of perphenazine was independent of dose and ranged between 9 and 12 hours. Perphenazine is metabolized hepatically.

It is contemplated that particular embodiments include perphenazine as the second component in combination with lithium. It is further contemplated that perphenazine is administered with lithium in a single formulation.

9. Molindone Hydrochloride

Molindone hydrochloride's chemical name is 3-ethyl-6,7-dihydro-2-methyl-5-(morpholinomethyl) indol-4 (5H)-one hydrochloride. It is commercially available as MOBAN (Endo) in 5, 10, 25, 50, and 100 mg tablets.

Molindone hydrochloride is indicated for the management of schizophrenia. It is contraindicated in severe central nervous system depression (alcohol, barbiturates, narcotics, etc.) or comatose states, and in patients with known hypersensitivity to the drug.

Maintenance dosages range from 5 mg to 15 mg three or four times a day for mild cases to 225 mg/day in severe instances. Human metabolic studies show molindone hydrochloride to be rapidly absorbed and metabolized when given orally. Unmetabolized drug reached a peak blood level at 1.5 hours.

It is contemplated that particular embodiments include molindone hydrochloride as the second component in combination with lithium. It is further contemplated that molindone hydrochloride is administered with lithium in a single formulation.

### F. Tricyclic Antidepressants

1. Desipramine

Desipramine's chemical name is 5H-Dibenz(b,f)azepine-5-propanamine, 10,11-dihydro-N-methyl-, monohydrochloride. Desipramine is commercially available under the brand names NORPRAMIN and PERTOFRANE in multiple dosages from many different suppliers.

Desipramine is indicated for the treatment of depressive illness, including the depressed phase of manic depressive disorder, involutional melancholia and psychotic depression. It may also be indicated in the management of depression of nonpsychotic degree such as in the selected cases of depressive neurosis. Desipramine is contraindicated for use with MAOIs. Although the dose for each patient may be individualized, the usual adult dose of desipramine is 100 to 200 mg per day, but may be as high as 300 mg/day.

It is contemplated that particular embodiments include desipramine as the third component in combination with lithium and an SSRI. In other embodiments, desipramine is administered in combination with lithium and an SSRI in a single formulation. It is also contemplated that desipramine is administered with lithium in a single formulation. In other embodiments, desipramine is administered in combination with bupropion in a single formulation.

2. Nortriptyline

Nortriptyline's chemical name is 1-Propanamine,3-(10,11-dihydro-5H-dibenzo (a,d)cyclohepten-5-ylidene)-N-methyl-, hydrochloride. Nortriptyline is a tricyclic antidepressant of the dibenzocyclohepten type and an active metabolite of amitriptyline. Nortriptyline is commercially available as AVENTYL and PAMELOR from various suppliers in multiple dosages.

Nortriptyline is indicated for use for depression and contraindicated for use with MAOIs. Dosages range from 25-250 mg/day. Peak plasma concentrations are obtained within 7 to 8.5 hours after oral administration. The plasma half-life of nortriptyline is between 16 and 90 hours. Nortriptyline is hepatically metabolized and excreted renally.

It is contemplated that particular embodiments include nortriptyline as the third component in combination with lithium and an SSRI. In other embodiments, nortriptyline is administered in combination with lithium and an SSRI in a single formulation. It is also contemplated that nortriptyline is administered with lithium in a single formulation. In other embodiments, nortriptyline is administered in combination with bupropion in a single formulation.

3. Amitriptyline Hydrochloride

Amitriptyline hydrochloride's chemical name is 3-(10,1-dihydro-5H-dibenzo[a,d]cycloheptene-5-ylidene)-N,N-dimethyl-1-propanamine hydrochloride. Amitriptyline is commercially available as ELAVIL (Zeneca) and ENDEP (Roche) in various dosages and forms.

Amitriptyline hydrochloride is indicated for relief of symptoms of depression; in particular, endogenous depression is more likely to be alleviated than other depressive states. It is contraindicated for use for people with hypersensitivity to it, and for use with MAOIs and CISAPRIDE (Propulsid). The usual adult outpatient dosage ranges from 50 mg to 150 mg/day, but hospitalized patients may require dosages as high as 300 mg/day. Lower dosages are recommended for adolescent and elderly patients; 10 mg three times a day with 20 mg at bedtime may be satisfactory.

Because of the wide variation in absorption and distribution of tricyclic antidepressants in body fluid, it is difficult to directly correlate plasma levels and therapeutic effect. Adjustments in dosage should be made according to the patient's clinical response, not on the basis of plasma levels, The half-life of amitriptyline is 24 hours. Amitriptyline hydrochloride is rapidly absorbed and metabolized.

It is contemplated that particular embodiments include amitriptyline hydrochloride as the second component in combination with bupropion in a single formulation.

4. Clomipramine Hydrochloride

Clomipramine hydrochloride's chemical name is 3-chloro-5-[3-(dimethylamino)propyl]-10,11-dihydro-5H-dibenz[b,f]azepine monohydrochloride. It is commercially available as ANAFRANIL (CibaGeneva and Novartis). Clomipramine hydrochloride is available in 25, 50, and 75 mg capsules.

Clomipramine hydrochloride is indicated for the treatment of obsessions and compulsions in patients with Obsessive-Compulsive Disorder (OCD). It is contraindicated in patients with a history of hypersensitivity to clomipramine or other tricyclic antidepressants. Clomipramine hydrochloride should not be given in combination, or within 14 days before or after treatment, with an MAOI. Clomipramine hydrochloride is also contraindicated during the acute recovery period after a myocardial infarction.

After a single 50 mg oral dose, maximum plasma concentrations occur within 2 to 6 hours. After a 150 mg dose, the half-life of clomipramine ranges from 19 hours to 37 hours. Metabolites are excreted in urine and feces, following biliary elimination.

It is contemplated that particular embodiments include clomipramine hydrochloride as the second component in combination with bupropion in a single formulation.

5. Doxepin Hydrochloride

Doxepin hydrochloride's chemical name is 1-Propanamine, 3-dibenz[b,e]oxepin-11(6H)ylidene-N,N-dimethyl-, hydrochloride. It is commercially available as SINEQUAN (Roerig) or ADAPIN (Fisons).

Doxepin hydrochloride is indicated for treatment of psychoneurotic patients with depression and/or anxiety, depression and/or anxiety associated with alcoholism or organic disease, or psychotic depressive disorders with associated anxiety including involutional depression and manic-depressive disorders. It is contraindicated for use in individuals who have shown hypersensitivity to the drug, and in patients with glaucoma or a tendency to urinary retention. MAOIs should be discontinued at least two weeks prior to the imitation of therapy with doxepin.

The usual optimum dose range is 75 to 150 mg/day; however, in more severely ill patients, higher doses may be required with subsequent gradual increase to 300 mg or more per day. Patients with very mild symptomatology or emotional symptoms accompanying organic disease may be controlled with on doses as low as 25 to 50 mg/day.

It is contemplated that particular embodiments include doxepin as the second component in combination with bupropion. It is further contemplated that doxepin is administered with bupropion in a single formulation.

6. Imipramine Pamoate

Imipramine pamoate's chemical name is 5-[3(dimethylamino)propyl]-10,11-dihydro-5H-dibenz[b,f]azepine 4,4′-methylenebis-(3-hydroxy-2-naphthoate). It is commercially available as TOFRANIL (Novartis) and JANIMINE (Abbott Laboratories).

Imipramine pamoate is indicated for use in the relief of symptoms of depression. Endogenous depression is more likely to be alleviated than other depressive states. It is contraindicated for use with MAOIs and during the acute recovery period alter a myocardial infarction. Patients with a known hypersensitivity to this compound should not be given the drug.

The recommended dosage for outpatients is 75 to 200 mg/day. Hospitalized patients may receive dosages up to 250 to 300 mg/day. In adolescent and geriatric patients, it is generally unnecessary to exceed 100 mg/day.

It is contemplated that particular embodiments include imipramine pamoate as the second component in combination with bupropion. It is further contemplated that imipramine pamoate is administered with bupropion in a single formulation.

7. Trimipramine maleate

Trimipramine maleate's chemical name is 5-(3-dimethylamino-2-methylpropyl)-10,11-dihydro-5H-dibenz (b,f) azepine acid maleate (racemic form). It is commercially available as SURMONTIL (Odyssey) in 25, 50, and 100 mg capsules.

Trimipramine maleate is indicated for the relief of symptoms of depression.

Endogenous depression is more likely to be alleviated than other depressive states. It is contraindicated in cases of known hypersensitivity to the drug and during the recovery period after a myocardial infarction. Trimipramine maleate is also contraindicated for use with MAOIs.

For adults, the usual dosage is 75 mg/day in divided doses, increased to 150 mg/day, but dosages over 200 mg/day are not recommended. In hospitalized patients, the dose may be increased to the maximum recommended dose of 250 to 300 mg/day.

It is contemplated that particular embodiments include trimipramine maleate as the second component in combination with bupropion. It is further contemplated that trimipramine maleate is administered with bupropion in a single formulation.

8. Protriptyline

Protriptyline's chemical name is N-methyl-5H-dibenzo[a,d]-cycloheptene-5-propanamine hydrochloride and it is commercially available as VIVACTIL (Merck). It is supplied as 5 mg and 10 mg film coated tablets.

Protriptyline has been found in some studies to have a more rapid onset of action than imipramine or amitriptyline. The initial clinical effect may occur within one week. Sedative and tranquilizing properties are lacking. Protriptyline is well absorbed from the gastrointestinal tract and is rapidly sequestered in tissues. Relatively low plasma levels are found after administration. Studies show significant plasma levels within 2 hours, peaking at 8 to 12 hours, then declining gradually. Urinary excretion studies showed significant amounts of radioactivity in 2 hours.

Protriptyline is indicated for all types of mental depression. It is also suitable for apathetic, withdrawn, depressed patients. It is contraindicated for use with MAOIs and in patients with known hypersensitivity to protriptyline. Protriptyline is also contraindicated during the acute recovery phase following myocardial infarction, and in the presence of acute congestive heart failure. Because it has anticholinergic activity, protriptyline is contraindicated in glaucoma and when there is predisposition to urinary retention.

The typical adult dosage is 15 to 40 mg a day divided into 3 or 4 doses. If necessary, dosage may be increased to 60 mg a day.

It is contemplated that particular embodiments include protriptyline as the second component in combination with bupropion in a single formulation.

### G. Tetracyclic Antidepressants 1. Amoxapine

Amoxapine is designated chemically as 2-Chloro-11-(1-piperazinyl)dibenz-[b,f][1,4]oxazepine. It is commercially available as ASENDIN. Amoxapine is supplied for oral administration as 25, 50, 100 and 150 mg tablets.

Amoxapine is indicated for the relief of symptoms of depression in patients with neurotic or reactive depressive disorders as well as endogenous and psychotic depressions. It is indicated for depression accompanied by anxiety or agitation. Amoxapine is contraindicated for use with MAOIs and in patients who have shown prior hypersensitivity to dibenzoxazepine compounds.

Amoxapine is absorbed rapidly and reaches peak blood levels approximately 90 minutes after ingestion. It is almost completely metabolized. The main route of excretion is the kidney. Amoxapine serum concentration declines with a half-life of eight hours. Usual effective dosage is 200 to 300 mg daily.

It is contemplated that particular embodiments include amoxapine as the second component in combination with bupropion in a single formulation.

2. Maprotiline

Maprotiline's chemical name is N-Methyl-9,10-ethanoanthracene-9(10H)-propanamine. It is commercially available as LUDIOMIL (Novartis). Maprotiline is supplied in 10, 25, 50, 75 mg tablets.

Maprotiline is indicated for treatment of endogenous depressive illness, including the depressed phase of manic-depressive illness (bipolar depression), psychotic depression (unipolar depression), and involutional melancholia. It might also be useful in selected patients suffering severe depressive neurosis. Maprotiline is contraindicated for use with a MAO inhibitor and in patients with a history of hypersensitivity to the drug, and in those with existing severe hepatic or renal damage or a history of severe blood dyscrasias. It is also contraindicated during the acute recovery phase following myocardial infarction in the presence of acute congestive heart failure.

In adults, the recommended initial dosage is 75 mg daily in 2 or 3 divided doses. The maximum recommended dose in outpatients is 150 mg daily, although doses up to 200 mg may be required in some patients. Severely depressed hospitalized patients may require a higher initial dose of 100 mg daily in 2 or 3 divided doses, and some may require up to 225 mg in divided doses.

Maprotiline is completely absorbed after oral administration. The half-life of unchanged maprotiline is relatively long and ranges from 27.4 to 57.6 hours. Maprotiline is metabolized by N-demethylation, deamination, aliphatic and aromatic hydroxylations and by formation of aromatic methoxy derivatives. It is excreted primarily in the urine.

It is contemplated that particular embodiments include maprotiline as the second component in combination with bupropion. It is further contemplated that maprotiline is administered with bupropion in a single formulation.

### H. Monoamine Oxidase Inhibitors (MAOI)

1. Phenelzine Sulfate

Phenelzine sulfate, (2-phenethyl)hydrazine, is a potent inhibitor of monoamine oxidase. Phenelzine sulfate is commercially available as NARDIL (Parke-Davis) in tablet form.

Following a single 30 mg dose of phenelzine sulfate, a mean peak plasma concentration was reached at 43 minutes. Phenelzine sulfate is extensively metabolized, primarily by oxidation via monoamine oxidase. The mean elimination half-life of as single 30 mg dose is 11.6 hours.

Phenelzine sulfate is indicated for use in depressed patients clinically characterized as “atypical,” “nonendogenous,” or “neurotic.” These patients often have mixed anxiety and depression and phobic or hypochondrial features. Phenelzine sulfate is contraindicated in those who are hypersensitive or have pheochromocytoma, congestive heart failure, a history of liver disease or abnormal liver function tests. Dosages typically range from 15-90 mg/day.

It is contemplated that embodiments include phenelzine sulfate combined with lithium in a single formulation for the treatment of mood spectrum disorder.

2. Tranylcypromine Sulfate

Tranylcypromine sulfate, (±)-trans-2-phenylcyclopropylamine sulfate, is a non-hydrazine monoamine oxidase inhibitor. It increases the concentration of epinephrine, norepinephrine, and serotonin in storage sites throughout the nervous system. Tranylcypromine sulfate is commercially available as PARNATE (Glaxo Smith Kline) in 10 mg tablets.

Tranylcypromine sulfate is indicated for use for treatment of Major Depressive Disorder without Melancholia. Tranylcypromine is contraindicated for use with other MAOI's or dibenzazepine derivatives; sympathomimetics such as amphetamines central nervous system depressants such as narcotics and alcohol, anti-hypertensive, diuretic, antihistaminic, sedative or anesthetic drugs; bupropion hydrochloride, buspirone hydrochloride, and dextromethorphan. Typical dosages range from 30-60 mg/day.

It is contemplated that embodiments include tranylcypromine sulfate combined with lithium in a single formulation for the treatment of mood spectrum disorder.

3. Selegiline

Selegiline's chemical name is (−)-(N)-Methyl-N-[(IR)-1-methyl-2-phenylethyl]prop-2-yn-1-amine. It is commercially available as EMSAM (Bristol-Myers Squibb).

Selegiline is indicated for the treatment of major depressive disorder. It is contraindicated in patients with known hypersensitivity to selegiline or to any component of the transdermal system. It is also contraindicated with selective serotonin reuptake inhibitors (SSRIs, for example, but not limited to, fluoxetine, sertraline, and paroxetine), dual serotonin and norepinephrine reuptake inhibitors (SNRIs, non-limiting examples include, venlafaxine and duloxetine), tricyclic antidepressants (TCAs, for example, but not limited to, imipramine and amitriptyline), bupropion hydrochloride; meperidine and analgesic agents such as tramadol, methadone and propoxyphene; the antitussive agent dextromethorphan; St. John's wort; mirtazapine; and cyclobenzaprine. The selegiline transdermal system should not be used with oral selegiline or other MAO inhibitors (MAOIs non-limiting examples include, isocarboxazid, phenelzine, and tranylcypromine). Carbamazepine and oxcarbazepine are contraindicated in patients taking selegiline. Selegiline is contraindicated for use with sympathomimetic amines, including amphetamines as well as cold products and weight-reducing preparations that contain vasoconstrictors (for example, but not limited to, pseudoephedrine, phenylephrine, phenylpropanolamine, and ephedrine).

The recommended starting dose and target dose for the selegiline transdermal system is 6 mg/24 hours. Steady-state selegiline plasma concentrations were achieved within five days of daily dosing.

It is contemplated that particular embodiments include selegiline as the second component in combination with lithium. It is further contemplated that selegiline is administered with lithium in a single formulation. It is also contemplated that selegiline and lithium are formulated into a single dose formulation in the form of a transdermal patch.

### Atypical Antidepressants

1. Nefazodone

The chemical name for nefazodone is 2-[3-[4-(3-chlorophenyl)-1-piperazinyl]propyl]-5-ethyl-2,4-dihydro-4-(2-phenoxyethyl)-3H-,1,2,4-triazo-3-one monohydrochloride. It is commercially available as SERZONE (Bristol-Myers Squibb). SERZONE is supplied as 50, 100, 150, 200, or 250 mg tablets.

Nefazodone is indicated for the treatment of major depressive disorder. Coadministration of terfenadine, astemizole, cisapride, pimozide, or carbamazepine with nefazodone is contraindicated. Nefazodone is also contraindicated in patients who were withdrawn from nefazodone because of evidence of liver injury and in patients who have demonstrated hypersensitivity to nefazodone, its inactive ingredients, or other phenylpiperazine antidepressants.

The recommended starting dose for nefazodone is 200 mg/day, administered in two divided doses. In the controlled clinical trials, the effective dose range was generally 300 to 600 mg/day. Peak plasma concentrations occur at about one hour and the half-life of nefazodone is 2-4 hours.

It is contemplated that particular embodiments include nefazodone as the second component in combination with lithium in a single formulation.

2. Trazodone

Trazodone's chemical name is 2-[3-[4-(3-chlorophenyl)-1-piperazinyl]propyl]-1,2,4-triazolo[4,3-a]pyridin-3(2H)-one. It is commercially available as DESYREL (Bristol-Myers Squibb), which is supplied for oral administration in 50, 100, 150, and 300 mg tablets.

Trazodone is indicated for the treatment of depression. The efficacy of trazodone has been demonstrated in both inpatient and outpatient settings and for depressed patients with and without prominent anxiety. It is contraindicated in patients hypersensitive to trazodone.

An initial dose of 150 mg/day in divided doses is suggested. The maximum dose for outpatients usually should not exceed 400 mg/day in divided doses. More severely depressed patients may be given up to 600 mg/day in divided doses. Peak plasma levels occur approximately one hour after dosing when trazodone is taken on an empty stomach or two hours after dosing when taken with food. It is contemplated that particular embodiments include trazodone as the second component in combination with lithium in a single formulation

### J. Others: Bupropion, Mirtazapine, Modafinil

1. Bupropion Hydrochloride

Bupropion hydrochloride, (±)-1-(3-chlorophenyl)-2-[(1,1-dimethylethyl)amino]-1-propanone hydrochloride is known and described in U.S. Pat. Nos. 3,819,706 and 3,885,046. Bupropion hydrochloride is commercially available as ZYBAN, WELLBUTRIN, WELLBUTRIN SR, and WELLBUTRIN XL (Glaxo Smith Kline) in multiple dosages.

Bupropion is a relatively weak inhibitor of the neuronal uptake of norepinephrine, serotonin, and dopamine and does not inhibit monoamine oxidase. Bupropion is a racemic mixture and follows biphasic pharmacokinetics. Peak plasma concentrations are reached within 3 hours. Bupropion is extensively metabolized with a mean elimination half-life of approximately 21 hours. Steady state plasma concentrations of bupropion and metabolites are reached within 5-8 days.

Bupropion hydrochloride is indicated for use as an aid to smoking cessation treatment and treatment of major depressive disorder. Bupropion is contraindicated in patients with seizure disorder, patients on any other bupropion containing medications, patients with a current or prior diagnosis of bulimia or anorexia nervosa, patients undergoing abrupt discontinuation of alcohol or sedatives (including benzodiazepines), patients on MAOIs, and those with allergies to bupropion.

Dosages for bupropion hydrochloride range from 150-450 mg/day. For patients with hepatic impairment, dosages should not exceed 150 mg/day. For patients with renal impairment, bupropion should be used with caution.

It is contemplated that particular embodiments include bupropion hydrochloride as the third component in combination with lithium and an SSRI. In other embodiments, bupropion hydrochloride is administered in combination with lithium and an SSRI in a single formulation. It is also contemplated that bupropion hydrochloride is administered with lithium in a single formulation. In further embodiments, it is contemplated that bupropion is administered in combination with a tricyclic antidepressant, tetracyclic antidepressant, or an antiseizure drug in a single formulation. In other embodiments, bupropion hydrochloride is administered in combination with lamotrigine and lithium in a single formulation. It is further contemplated that bupropion hydrochloride is administered in combination with lamotrigine in a single formulation. It is also contemplated that bupropion hydrochloride is administered in combination with lithium and modafinil in a single formulation.

2. Mirtazapine

Mirtazapine, 1,2,3,4,10,14b-hexahydro-2-mehtylpryazino [2.1-a] pyrido [2,3-c] benzazepine is a racemic tetracyclic compound that belongs to the piperaminoazepine group of compounds. Mirtazapine is commercially available as REMERON (Organon) in 15, 30, and 45 mg tablets.

Mirtazapine acts as an antagonist at central presynaptic a2 adrenergic inhibitory autoreceptors and heteroreceptors. Mirtazapine is a potent antagonist of serotonin 5-HT₂ and 5-HT₃ receptors and has no significant affinity for 5-HT_{1A} and 5-HT_{1B} receptors. Mirtazapine is also a potent antagonist of histamine H1 receptors. Mirtazapine is a moderate antagonist at peripheral al adrenergic and muscarinic receptors.

Mirtazapine is rapidly absorbed following oral administration and has a half-life of approximately 20-40 hours. Peak plasma concentrations are reached within 2 hours following an oral dose. Mirtazapine is extensively metabolized by demethylation and hydroxylation followed by glucuronide conjugation. The (−) enantiomer has an elimination half-life that is approximately twice as long at the (+) enantiomer and therefore achieves plasma levels that are about 3× as high as that of the (+) enantiomer. Mirtazapine clearance is decreased in patients with moderate to severe renal impairment. Mirtazapine is indicated for treatment of major depressive disorder. Typical dosages range from 15-45 mg/day.

It is contemplated that embodiments include mirtazapine combined with lithium in a single formulation for the treatment of mood spectrum disorder.

3. Modafinil

Modafinil is a wakefulness-promoting agent for oral administration. The chemical name for modafinil is 2-[(diphenylmethyl)sulfinyl]acetamide. It is commercially available as PROVIGIL (Cephalon) in 100 or 200 mg tablets.

Modafinil is indicated to improve wakefulness in patients with excessive sleepiness associated with narcolepsy, obstructive sleep apnea/hypopnea syndrome, and shift work sleep disorder. It is contraindicated in patients with known hypersensitivity to modafinil. The recommended dose of modafinil is 200 mg given once a day.

Absorption of modafinil tablets is rapid, with peak plasma concentrations occurring at 2-4 hours. The major route of elimination (−90%) is metabolism, primarily by the liver, with subsequent renal elimination of the metabolites. It is contemplated that particular embodiments include modafinil in combination with bupropion and lithium. It is further contemplated that modafinil is administered with bupropion and lithium in a single formulation.

### IV. Combinations

The current invention describes a mood stabilizer, SSRIs, antiseizure drugs, atypical neuroleptics, tricyclic antidepressants, heterocyclic antidepressants and others, all of which could be combined in thousands of possible drug doses, order of administration to the patient and timing of dosage changes. The present invention represents a simplified, yet clinically effective treatment that reduces medication choices to a manageable clinical regimen. Traditionally, psychiatry has not favored fixed dosed drug combinations because of the inherent inflexibility of dosing and the simultaneous administration of two drugs. Three drug combinations are even more complex to manage. There are many problems with the simultaneous initiation of treatment with three drugs. Specifically, it would be difficult to separate out both beneficial effects and side effects. Fixed dosing and simultaneous administration make both the achievement of therapeutic efficacy and the elimination of side effects more challenging. Psychiatrists are highly critical of any fixed dose treatment, even one utilizing progressive dosage increases and careful symptom monitoring to titrate both target and maintenance dosing. Embodiments described herein, however, permit individualized flexibility in each patient's specific drug combination. Screening for mood disorders by PCPs will not only lead to treatment of a heretofore untreated population, but also will increase referrals to psychiatrists.

A. Lithium+SSRI

Lithium alone is a poor antidepressant. Lithium, however, is a “gold standard” mood stabilizer and anti-manic agent. Citalopram, an SSRI, is a very effective antidepressant. In embodiments provided herein, lithium is combined with an SSRI in a single formulation. In particular embodiments, lithium and citalopram, for example, are combined into a single formulation wherein the drugs work in tandem to treat acute and chronic mood disorder. The embodiment that is the combination of lithium with citalopram is hereinafter referred to as SERATROL.

Traditionally, Bipolar I, manic type, could initially be managed with lithium alone, or perhaps with the assistance of an atypical neuroleptic like quetiapine fumarate. Long-term maintenance of all bipolar sub-types, however, may include an antidepressant, such as an SSRI, as manic patients will not remain medication compliant without the maintenance addition of a powerful antidepressant.

Lithium and SSRIs are highly synergistic drugs in the treatment of mood disorder. Lithium augments the SSRIs antidepressant effect while counteracting any possible hypomanic side effects that the SSRI might theoretically induce (antidepressants do not stabilize mood and may even act as a mood destabilizer), particularly in undiagnosed bipolar depression sufferers. An example might be a depressed patient who is given an antidepressant and who then cycles into mania.

The SSRIs disclosed herein, make lithium a much better antidepressant and confers powerful treatment efficacy for mood spectrum disorders such as, but not limited to, MDD, OCD, GAD, SAD, PD, PMDD, SUD, BPD, bipolar disorder, phobias, substance use disorders as described above. Studies have shown that lithium increases the serotonergic effects of citalopram. Carli, et al., Neurochem Res. 22 (4):427-35 (1997).

Embodiments of the present invention include lithium with an SSRI. Particular embodiments include lithium (including extended release formulations) in combination with citalopram, escitalopram, paroxetine, sertraline, fluoxetine, or venlafaxine. In yet further embodiments, in an individualized dosage formulation, lithium may range from 150 mg to 1500 mg per dose per day. In yet further embodiments, Table 1 provides examples of ranges of dosages of SSRIs that can be combined with lithium in a single formulation for the treatment of mood spectrum disorders as described herein. Table 2 provides a particular example of the various formulations of lithium and citalopram (SERATROL).

TABLE 1
Composition of Lithium with an SSRI, Examples of SSRI Dosages
to be Combined with 150-1500 mg/day Lithium.

| | Lithium: Daily Dosage X150-1500 mg/day | |
|---|---|---|
| | to provide plasma levels of 0.6-1.2 | |
| | mEQ/L Li to be combined with | |
| SSRI | the dosage of SSRI listed below | |
| Paroxetine | 10-100 | mg/day |
| Paroxetine CR | 12.5-37.5 | mg/day |
| Sertraline | 25-250 | mg/day |
| Fluoxetine | 5-80 | mg/day |
| Escitalopram | 5-30 | mg/day |
| Citalopram | 10-360 | mg/day |
| Venlafaxine | 37.5-300 | mg/day |
| Venlafaxine XR | 37.5-300 | mg/day |
| Duloxetine (SSRI) | 30-60 | mg/day |

TABLE 2
Matrix of Lithium Dosages and Citalopram Dosages to Make
Exemplary Formulations of SERATROL

| | 150 mg | 300 mg | 450 mg | 600 mg | 750 mg | 900 mg | 1050 mg | 1200 mg |
|---|---|---|---|---|---|---|---|---|
| | Li | Li | Li | Li | Li | Li | Li | Li |
| 10 mg | 150/10 | 300/10 | 450/10 | 600/10 | 750/10 | 900/10 | 1050/10 | 1200/10 |
| Citalop. | | | | | | | | |
| 20 mg | 150/20 | 300/20 | 450/20 | 600/20 | 750/20 | 900/20 | 1050/20 | 1200/20 |
| Citalop. | | | | | | | | |
| 30 mg | 150/30 | 300/30 | 450/30 | 600/30 | 750/30 | 900/30 | 1050/30 | 1200/30 |
| Citalop. | | | | | | | | |
| 40 mg | 150/40 | 300/40 | 450/40 | 600/40 | 750/40 | 900/40 | 1050/40 | 1200/40 |
| Citalop. | | | | | | | | |
| 50 mg | 150/50 | 300/50 | 450/50 | 600/50 | 750/50 | 900/50 | 1050/50 | 1200/50 |
| Citalop. | | | | | | | | |
| 60 mg | 150/60 | 300/60 | 450/60 | 600/60 | 750/60 | 900/60 | 1050/60 | 1200/60 |
| Citalop. | | | | | | | | |
| 70 mg | 150/70 | 300/70 | 450/70 | 600/70 | 750/70 | 900/70 | 1050/70 | 1200/70 |
| Citalop. | | | | | | | | |
| 80 mg | 150/80 | 300/80 | 450/80 | 600/80 | 750/80 | 900/80 | 1050/80 | 1200/80 |
| Citalop. | | | | | | | | |

In yet further embodiments, for example, lithium (dose range 150-1500 mg with 150 mg intervals, as in Table 2) can be combined with escitalopram doses of 5, 10, 20 and 30 mg. This matrix (Table 3) is provided as an example of a further embodiment of lithium and an SSRI in a single formulation.

TABLE 3
Matrix of Lithium Dosages and Escitalopram Dosages to Make
Exemplary Formulations

| | 5 mg | | | |
|---|---|---|---|---|
| | Escital. | 10 mg Escital. | 20 mg Escital. | 30 mg Escital. |
| 150 mg Li | 150/5 | 150/10 | 150/20 | 150/30 |
| 300 mg Li | 300/5 | 300/10 | 300/20 | 300/30 |
| 450 mg Li | 450/5 | 450/10 | 450/20 | 450/30 |
| 600 mg Li | 600/5 | 600/10 | 600/20 | 600/30 |
| 750 mg Li | 750/5 | 750/10 | 750/20 | 750/30 |
| 900 mg Li | 900/5 | 900/10 | 900/20 | 900/30 |
| 1050 mg Li | 1050/5 | 1050/10 | 1050/20 | 1050/30 |
| 1200 mg Li | 1200/5 | 1200/10 | 1200/20 | 1200/30 |
| 1350 mg Li | 1350/5 | 1350/10 | 1350/20 | 1350/30 |
| 1500 mg Li | 1500/5 | 1500/10 | 1500/20 | 1500/30 |

Other embodiments of lithium combined with an SSRI in a single formulation include the already recited dosages of lithium combined with 10-80 mg of paroxetine. For example, but not limited to, the 10 doses of lithium can be combined with the following paroxetine dosages: 10, 20, 30, 40, 50, 60, 70, and 80 mg. Alternatively, paroxetine continued release (“CR”) can also be used at dosages of 12.5, 25 and 37.5 mg. Similarly, further embodiments include lithium combined with sertraline in a single formulation. For example, but not limited to, the 10 doses of lithium can be combined with the following sertraline doses: 25, 50, 75, 100, 125, 150, 175, 200, 225, and 250 mg. Lithium may also be combined fluoxetine in another embodiment using a range of 10 to 80 mg of fluoxetine. Exemplary dosages of fluoxetine to combine with lithium are: 10, 20, 30, 40, 50, 60, 70, and 80 mg. In yet another embodiment, varied doses of lithium can be combined with venlafaxine or venlafaxine XR in the following non-limiting exemplary dosages of 37.5, 75, 150, and 225 mg.

### B. Lithium+SSRI+Antiseizure

In addition to the combination of lithium and an SSRI, a third component can also be added for the treatment of mood spectrum disorder. In particular embodiments, the third component is an antiseizure medication. In yet further embodiments, the antiseizure medication can be combined with the lithium and SSRI into a single formulation or, alternatively, the antiseizure medication can be co-administered with a single formulation of lithium with an SSRI. In another embodiment, lithium can be combined with an antiseizure drug without an SSRI.

For example, in a particular embodiment, SERATROL can be co-administered with lamotrigine in the treatment of bipolar illness. In another embodiment, lithium, citalopram and lamotrigine can be combined into a single formulation. Alternatively, lithium may be combined with lamotrigine without citalopram.

Table 4 provides non-limiting examples of antiseizure medications that can be combined with lithium and an SSRI either as a co-administered drug or in combination with lithium and the SSRI. The antiseizure medications may also be combined with lithium without an SSRI.

TABLE 4
Examples of Antiseizure Medications and Dosage Ranges

| Antiseizure Medication | Dosage Ranges |
|---|---|
| Oxcarbazepine | 300-1500 mg/day |
| Lamotrigine | 25-400 mg/day |
| Topiramate | 25-400 mg/day |
| Divalproex | 250-2400 mg/day |
| Levetiracetam | 250-2400 mg/day |
| Gabapentin | 100-2400 mg/day |
| Tiagabine | 2-16 mg/day |
| Zonisamide | 100-600 mg/day |

In embodiments described herein, any of the disclosed or described combinations of lithium with an SSRI can be combined with an antiseizure medication. In particular embodiments, any of the disclosed or described combinations of lithium with an SSRI can be combined with oxcarbazepine in the following non-limiting dosages: 300, 600, 900, 1200, and 1500 mg. These non-limiting dosages may be combined in either a single formulation with lithium and an SSRI or co-administered with the combination of lithium and an SSRI. Alternatively, these dosages may be combined with lithium alone in a single formulation. Other examples of ranges and similar combinations to that described for oxcarbazepine, providing further embodiments, include: (1) lamotrigine in the following dosages: 25, 50, 75, 100, 150, 200, 250, 300, 350, and 400 mg; (2) topiramate in the following dosages: 25, 50, 75, 100, 150, 200, 250, 300, 350, and 400 mg; (3) divalproex sodium in the following dosages: 250, 500, 750, 1000, 1250, 1500, and 1750 mg; (4) levetiracetam in the following dosages: 250, 500, 750, 1000, 1250, 1500, 1750, 2000, 2250, and 2500 mg; (5) gabapentin in the following dosages: 100, 400, 800,1200, 1600, and 2400 mg (6) tiagabine in the following dosages: 2, 4, 8, 12, and 16 mg; and (7) zonisamide in the following dosages: 100, 200, 400, and 600 mg.

Table 5 provides an example of particular dosages of SERATROL combined with or co-administered with exemplary dosages of oxcarbazepine. In further embodiments, other dosages of SERATROL may be combined with or co-administered with dosages of other antiseizure medications as disclosed herein.

TABLE 5
Example of Dosages of Oxcarbazepine Combined or Co-
Administered with Some Exemplary Dosages of SERATROL

| | 300 mg | 600 mg | 900 mg | 1200 mg | |
|---|---|---|---|---|---|
| | Oxc. | Oxc. | Oxc. | Oxc. | 1500 Oxc. |
| 150/10 | 150/10/300 | 150/10/600 | 150/10/900 | 150/10/1200 | 150/10/1500 |
| SERATROL | | | | | |
| 300/20 | 300/20/300 | 300/20/600 | 300/20/900 | 300/20/1200 | 300/20/1500 |
| SERATROL | | | | | |
| 450/30 | 450/30/300 | 450/30/600 | 450/30/900 | 450/30/1200 | 450/30/1500 |
| SERATROL | | | | | |
| 600/40 | 600/40/300 | 600/40/600 | 600/40/900 | 600/40/1200 | 600/40/1500 |
| SERATROL | | | | | |
| 750/50 | 750/50/300 | 750/50/600 | 750/40/900 | 750/40/1200 | 750/40/1500 |
| SERATROL | | | | | |

In another embodiment, antiseizure medications may be combined in a single formulation with lithium. Table 6 provides non-limiting examples of particular embodiments combining lithium with oxcarbazepine. Other embodiments include, but are not limited to, combining lithium with lamotrigine, topiramate, divalproex sodium or levetiracetam in the dosages provided herein as exemplified with oxcarbazepine.

TABLE 6
Exemplary Dosages of Oxcarbazepine Combined with Exemplary
Dosages of Lithium

| | 300 mg | 600 mg | 900 mg | 1200 mg | |
|---|---|---|---|---|---|
| | Oxc. | Oxc. | Oxc. | Oxc. | 1500 Oxc. |
| 150 | 150/300 | 150/600 | 150/900 | 150/1200 | 150/1500 |
| mg Li | | | | | |
| 300 | 300/300 | 300/600 | 300/900 | 300/1200 | 300/1500 |
| mg Li | | | | | |
| 450 | 450/300 | 450/600 | 450/900 | 450/1200 | 450/1500 |
| mg Li | | | | | |
| 600 | 600/300 | 600/600 | 600/900 | 600/1200 | 600/1500 |
| mg Li | | | | | |
| 750 | 750/300 | 750/600 | 750/900 | 750/1200 | 750/1500 |
| mg Li | | | | | |
| 900 | 900/300 | 900/600 | 900/900 | 900/1200 | 900/1500 |
| mg Li | | | | | |
| 1050 | 1050/300 | 1050/600 | 1050/900 | 1050/1200 | 1050/1500 |
| mg Li | | | | | |
| 1200 | 1200/300 | 1200/600 | 1200/900 | 1200/1200 | 1200/1500 |
| mg Li | | | | | |

### C. Lithium+SSRI +Atypical Neuroleptic

Another embodiment described herein includes atypical neuroleptics as the third component, wherein the first component is lithium and the second component is an SSRI for the treatment of mood spectrum disorder. In yet further embodiments, the atypical neuroleptic can be combined with the lithium and an SSRI into a single formulation or, alternatively, the atypical neuroleptic can be co-administered with a single formulation of lithium with an SSRI. In another embodiment, lithium can be combined with an atypical neuroleptic without an SSRI.

Particular embodiments, which include all three components, lithium, an SSRI and an atypical neuroleptic, are effective for treating Bipolar I Disorder, manic and mixed types, Schizoaffective disorder, mood spectrum disorder, as well as for treating aggressive manics who were previously being treated with just a lithium/atypical neuroleptic combination. For example, in a particular embodiment wherein the combination is lithium, citalopram and any atypical neuroleptic, the combination is effective for treating mood disorder. In another embodiment, wherein the atypical neuroleptic is quetiapine fumarate, which is combined with lithium and an SSRI, its use is effective for treating Bipolar and Schizoaffective disorder of all types in acute and maintenance phases. This particular embodiment is also useful in treating insomniacs with mood spectrum disorder and avoids use of addictive benzodiazepines.

Table 7 provides non-limiting examples of atypical neuroleptics that can be combined with lithium and an SSRI either as a co-administered drug or in combination with lithium and the SSRI. The atypical neuroleptics may also be combined with lithium without an SSRI.

TABLE 7
Examples of Atypical Neuroleptics and Dosage Ranges

| Atypical Neuroleptic | DoseRanges | |
|---|---|---|
| Aripiprazole | 2.5-30 | mg/day |
| Quetiapine Fumarate | 25-800 | mg/day |
| Risperidone | 0.5-6 | mg/day |
| Ziprasidone | 20-240 | mg/day |
| Olanzapine | 2.5-20 | mg/day |

Table 8 provides a non-limiting example of an atypical neuroleptic, aripiprazole, combined with some exemplary non-limiting dosages of lithium with citalopram. The atypical neuroleptic may be combined with lithium and the SSRI as a single dosage formulation or administered in conjunction with SERATROL.

TABLE 8
Exemplary Dosages of Aripiprazole Combined with Exemplary
Dosages of Lithium and Citalopram

| | 5 mg | 10 mg | 15 mg | 20 mg | 25 mg | 30 mg |
|---|---|---|---|---|---|---|
| | Ari. | Ari | Ari. | Ari. | Ari. | Ari. |
| 150/10 | 150/10/5 | 150/10/10 | 150/10/15 | 150/10/20 | 150/10/25 | 150/10/30 |
| SERATROL | | | | | | |
| 300/20 | 300/20/5 | 300/20/10 | 300/20/15 | 300/20/20 | 300/20/25 | 300/20/30 |
| SERATROL | | | | | | |
| 450/30 | 450/30/5 | 450/30/10 | 450/30/15 | 450/30/20 | 450/30/25 | 450/30/30 |
| SERATROL | | | | | | |
| 600/40 | 600/40/5 | 600/40/10 | 600/40/15 | 600/30/20 | 600/30/25 | 600/30/30 |
| SERATROL | | | | | | |
| 750/50 | 750/50/5 | 750/50/10 | 750/50/15 | 750/50/20 | 750/50/25 | 750/50/30 |
| SERATROL | | | | | | |
| 900/60 | 900/60/5 | 900/60/10 | 900/60/15 | 900/60/20 | 900/60/25 | 900/60/30 |
| SERATROL | | | | | | |

In yet further embodiments, other atypical neuroleptics as described herein, may also be combined as the third component with lithium and an SSRI or with lithium alone. Particular embodiments include quetiapine fumarate, risperidone, ziprasidone, and olanzapine. Exemplary non-limiting dosages for each atypical neuroleptic for combination are the following: (1) quetiapine fumarate: 25, 50, 75, 100, 150, 200, 250, 300, 350, 400, 450, 500, 550, 600, 650, 700, 750, and 800 mg; (2) risperidone: 0.5, 1, 2, 3, 4, 5, and 6 mg; (3) ziprasidone: 20, 40, 60, 80, 100, 120, 140, 160, 180, 200, 220, and 240 mg; and (4) olanzapine: 2.5, 5, 7.5, 10, 15, and 20 mg.

In a particular embodiment and non-limiting example, quetiapine fumarate is co-administered with lithium and an SSRI. In this particular embodiment, this combination is used to treat manic, hypomanic or agitated patients on as needed basis. The patient would already be on maintenance therapy with the lithium/SSRI combination. Quetiapine would be added to the regimen at 25 mg/day. The dosage of quetiapine can then be increased to 200 mg/day after the 2^{nd} week and up to 400 mg/day during weeks 6-8, and finally up to 600 mg/day.

In alternative embodiments, the atypical neuroleptic may be combined with lithium without the combination of an SSRI. The combination of an atypical neuroleptic with lithium is useful in treating aggressive manics, acute and maintenance indications for mood spectrum disorder or affective disorder. For example, quetiapine with lithium is an effective mood stabilizer.

Table 9 provides a non-limiting example of an atypical neuroleptic in combination with lithium without an SSRI in a single formulation. In these particular embodiments, non-limiting exemplary dosages of olanzapine are combined with non-limiting exemplary dosages of lithium. Other embodiments include, but are not limited to lithium in combination with aripiprazole, quetiapine fumarate, or risperidone in a single formulation.

TABLE 9
Exemplary Dosages of Olanzapine Combined with Exemplary
Dosages of Lithium

| | 2.5 mg | | | 10 mg | 15 mg | 20 mg |
|---|---|---|---|---|---|---|
| | Ol. | 5 mg Ol. | 7.5 mg Ol. | Ol. | Ol. | Ol. |
| 150 mg | 150/2.5 | 150/5 | 150/7.5 | 150/10 | 150/15 | 150/20 |
| Li | | | | | | |
| 300 mg | 300/2.5 | 300/5 | 300/7.5 | 300/10 | 300/15 | 300/20 |
| Li | | | | | | |
| 450 mg | 450/2.5 | 450/5 | 450/7.5 | 450/10 | 450/15 | 450/20 |
| Li | | | | | | |
| 600 mg | 600/2.5 | 600/5 | 600/7.5 | 600/10 | 600/15 | 600/20 |
| Li | | | | | | |
| 750 mg | 750/2.5 | 750/5 | 750/7.5 | 750/10 | 750/15 | 750/20 |
| Li | | | | | | |
| 900 mg | 900/2.5 | 900/5 | 900/7.5 | 900/10 | 900/15 | 900/20 |
| Li | | | | | | |
| 1050 mg | 1050/2.5 | 1050/5 | 1050/7.5 | 1050/10 | 1050/15 | 1050/20 |
| Li | | | | | | |
| 1200 mg | 1200/2.5 | 1200/5 | 1200/7.5 | 1200/10 | 1200/15 | 1200/20 |
| Li | | | | | | |

### D. Lithium+SSRI+Antipsychotic

In addition to the combination of lithium and an SSRI, a third component can also be added for the treatment of mood spectrum disorder. In particular embodiments, the third component is an antipsychotic medication. In yet further embodiments, the antipsychotic medication can be combined with lithium and an SSRI into a single formulation. Alternatively, the antipsychotic can be co-administered separately from the single formulation of lithium and an SSRI. In another embodiment, lithium can be combined with the antipsychotic medication without the presence of an SSRI. Then at the clinically appropriate time, citalopram could be added to the lithium/antipsychotic combination to prevent the emergence of or recycling into depression.

For example, long-acting haloperidol or fluphenazine could be combined with SERATROL to treat medication noncompliant mood disordered patients. In one embodiment, the combination can be the three drugs in a single formulation. In an alternative embodiment, the SERATROL is co-administered with haloperidol or fluphenazine as two separate drugs.

Table 10 provides non-limiting examples of antipsychotic medications that can be combined with lithium and an SSRI either as a co-administered drug or in combination with lithium and an SSRI. The antipsychotics may also be combined with lithium without an SSRI.

TABLE 10
Examples of Antipsychotic Medications and Dosage Ranges

| Antipsychotic | Dosage Ranges |
|---|---|
| Haloperidol | 0.5-40 mg/day |
| Fluphenazine | 0.5-40 mg/day |

In embodiments described herein, any of the disclosed or described combinations of lithium with an SSRI can be combined with an antipsychotic. In particular embodiments, examples of non-limiting dosages of haloperidol, or alternatively, fluphenazine, that can be combined as described herein include the following: 0.5, 1, 2, 4, 6, 8, 10, 15, 20, 25, 30, 35, and 40 mg. In particular embodiments, these non-limiting dosages can be combined in either a single formulation with lithium and an SSRI or co-administered with the combination of lithium and an SSRI. In alternative embodiments, these dosages of haloperidol or fluphenazine may be combined with lithium without an SSRI.

Table 11 shows some non-limiting examples of haloperidol combined with SERATROL. In yet further embodiments, other dosages of SERATROL or antipsychotic medication can be combined with or co-administered as disclosed herein.

TABLE 11
Example of Some Dosages of Haloperidol Combined or
Co-Administered with Some Exemplary Dosages of SERATROL

| | 0.5 mg Halo. | 2 mg Halo. | 10 mg Halo. | 20 mg Halo. | 30 mg Halo. |
|---|---|---|---|---|---|
| 150/10 | 150/10/0.5 | 150/10/2 | 150/10/10 | 150/10/20 | 150/10/30 |
| SERATROL | | | | | |
| 300/20 | 300/20/0.5 | 300/20/2 | 300/20/10 | 300/20/20 | 300/20/30 |
| SERATROL | | | | | |
| 450/30 | 450/30/0.5 | 450/30/2 | 450/30/10 | 450/30/20 | 450/30/30 |
| SERATROL | | | | | |
| 600/40 | 600/400/0.5 | 600/40/2 | 600/40/10 | 600/40/20 | 600/40/30 |
| SERATROL | | | | | |
| 750/50 | 750/50/0.5 | 750/50/2 | 750/50/10 | 750/50/20 | 750/50/30 |
| SERATROL | | | | | |

1. Lithium+Antipsychotic

In other embodiments, antipsychotic medications may be combined in a single formulation with lithium without an SSRI for the treatment of mood spectrum disorder. In certain embodiments, the combination of lithium and an antipsychotic is effective in the treatment of Bipolar I Disorder, depressed, manic, and mixed types, in both acute and maintenance phases, Borderline Personality Disorder, Cyclothymia, Dysthymia, Depressive Personality Disorder, Substance Use Disorder, and Treatment Resistant Depression in both acute and maintenance phases. In a particular embodiment, where the antipsychotic drug is haloperidol, the combination is effective for treatment of Bipolar I Disorder, manic type.

Table 12 provides non-limiting examples of antipsychotic medications and typical dosage ranges that may be combined with lithium.

TABLE 12
Examples of Antipsychotic Medications and Dosage Ranges

| Antipsychotic Medications | Dosage Ranges |
|---|---|
| Fluphenazine | 2-20 mg |
| Chlorpromazine hydrochloride | 25-600 mg |
| Thioridazine hydrochloride | 25-600 mg |
| Trifluoperazine hydrochloride | 1-20 mg |
| Thiothixene hydrochloride | 2-30 mg |
| Loxapine succinate | 10-100 mg |
| Perphenazine | 2-40 mg |
| Molindone hydrochloride | 10-100 mg |

Table 13 provides non-limiting examples of particular embodiments combining lithium with fluphenazine. Other embodiments include combining lithium with haloperidol.

TABLE 13
Exemplary Dosages of Fluphenazine Combined with Exemplary
Dosages of Lithium

| | 2 mg | 6 mg | 8 mg | | | |
|---|---|---|---|---|---|---|
| | Fl. | Fl. | Fl. | 10 mg Fl. | 15 mg Fl. | 20 mg Fl. |
| 150 | 150/2 | 150/6 | 150/8 | 150/10 | 150/15 | 150/20 |
| mg Li | | | | | | |
| 300 | 300/2 | 300/6 | 300/8 | 300/10 | 300/15 | 300/20 |
| mg Li | | | | | | |
| 450 | 450/2 | 450/6 | 450/8 | 450/10 | 450/15 | 450/20 |
| mg Li | | | | | | |
| 600 | 600/2 | 600/6 | 600/8 | 600/10 | 600/15 | 600/20 |
| mg Li | | | | | | |
| 750 | 750/2 | 750/6 | 750/8 | 750/10 | 750/15 | 750/20 |
| mg Li | | | | | | |
| 900 | 900/2 | 900/6 | 900/8 | 900/10 | 900/15 | 900/20 |
| mg Li | | | | | | |
| 1050 | 1050/2 | 1050/6 | 1050/8 | 1050/10 | 1050/15 | 1050/20 |
| mg Li | | | | | | |
| 1200 | 1200/2 | 1200/6 | 1200/8 | 1200/10 | 1200/15 | 1200/20 |
| mg Li | | | | | | |

Table 14 provides an example of particular dosages of chlorpromazine hydrochloride combined with lithium in single formulations. In particular embodiments, the following non-limiting dosages of chlorpromazine hydrochloride can be combined with lithium: 25, 100, 200, 300, 400, 500, 600 mg.

TABLE 14
Exemplary Dosages of Chlorpromazine Hydrochloride Combined
with Exemplary Dosages of Lithium (Dosages in bold print indicate maintenance
doses)

| | 25 mg | 100 mg | 200 mg | 300 mg | 400 mg | 500 mg | 600 mg |
|---|---|---|---|---|---|---|---|
| | Chlor. | Chlor. | Chlor. | Chlor. | Chlor. | Chlor. | Chlor. |
| 50 mg | 50/25 | 50/100 | 50/200 | 50/300 | 50/400 | 50/500 | 50/600 |
| Li | | | | | | | |
| 300 mg | 300/25 | 300/100 | 300/200 | 300/300 | 300/400 | 300/500 | 300/600 |
| Li | | | | | | | |
| 600 mg | 600/25 | 600/100 | 600/200 | 600/300 | 600/400 | 600/500 | 600/600 |
| Li | | | | | | | |
| 750 mg | 750/25 | 750/100 | 750/200 | 750/300 | 750/400 | 750/500 | 750/600 |
| Li | | | | | | | |
| 900 mg | 900/25 | 900/100 | 900/200 | 900/300 | 900/400 | 900/500 | 900/600 |
| Li | | | | | | | |
| 1200 mg | 1200/25 | 1200/100 | 1200/200 | 1200/300 | 1200/400 | 1200/500 | 1200/600 |
| Li | | | | | | | |

Table 15 provides an example of particular dosages of thioridazine hydrochloride combined with lithium in single formulations. In particular embodiments, the following non-limiting dosages of thioridazine hydrochloride can be combined with lithium: 25, 100, 200, 300, 400, 500, 600 mg.

TABLE 15
Exemplary Dosages of Thioridazine Hydrochloride Combined
with Exemplary Dosages of Lithium (Dosages in bold print indicate maintenance
doses)

| | 25 mg | 100 mg | 200 mg | 300 mg | 400 mg | 500 mg | 600 mg |
|---|---|---|---|---|---|---|---|
| | Thior. | Thior. | Thior. | Thior. | Thior. | Thior. | Thior. |
| 50 mg | 50/25 | 50/100 | 50/200 | 50/300 | 50/400 | 50/500 | 50/600 |
| Li | | | | | | | |
| 300 mg | 300/25 | 300/100 | 300/200 | 300/300 | 300/400 | 300/500 | 300/600 |
| Li | | | | | | | |
| 600 mg | 600/25 | 600/100 | 600/200 | 600/300 | 600/400 | 600/500 | 600/600 |
| Li | | | | | | | |
| 750 mg | 750/25 | 750/100 | 750/200 | 750/300 | 750/400 | 750/500 | 750/600 |
| Li | | | | | | | |
| 900 mg | 900/25 | 900/100 | 900/200 | 900/300 | 900/400 | 900/500 | 900/600 |
| Li | | | | | | | |
| 1200 mg | 1200/25 | 1200/100 | 1200/200 | 1200/300 | 1200/400 | 1200/500 | 1200/600 |
| Li | | | | | | | |
| 1500 mg | 1500/25 | 1500/100 | 1500/200 | 1500/300 | 1500/400 | 1500/500 | 1500/600 |
| Li | | | | | | | |

Table 16 provides an example of particular dosages of trifluoperazine hydrochloride combined with lithium in single formulations. In particular embodiments, the following non-limiting dosages of trifluoperazine hydrochloride can be combined with lithium: 1, 5, 15, 20 mg.

TABLE 16
Exemplary Dosages of Trifluoperazine Hydrochloride Combined
with Exemplary Dosages of Lithium (Dosages in bold print
indicate maintenance doses)

| | 1 mg Trif. | 5 mg Trif. | 15 mg Trif. | 20 mg Trif. |
|---|---|---|---|---|
| 50 mg Li | 50/1 | 50/5 | 50/15 | 50/20 |
| 300 mg Li | 300/1 | 300/5 | 300/15 | 300/20 |
| 600 mg Li | 600/1 | 600/5 | 600/15 | 600/20 |
| 750 mg Li | 750/1 | 750/5 | 750/15 | 750/20 |
| 900 mg Li | 900/1 | 900/5 | 900/15 | 900/20 |
| 1200 mg Li | 1200/1 | 1200/5 | 1200/15 | 1200/20 |
| 1500 mg Li | 1500/1 | 1500/5 | 1500/15 | 1500/20 |

Table 17 provides an example of particular dosages of thiothixene hydrochloride combined with lithium in single formulations. In particular embodiments, the following non-limiting dosages of thiothixene hydrochloride can be combined with lithium: 2, 5, 15, 20, 25, 30 mg.

TABLE 17
Exemplary Dosages of Thiothixene Hydrochloride Combined with
Exemplary Dosages of Lithium (Dosages in bold print indicate maintenance doses)

| | | 5 mg | 15 mg | 20 mg | 25 mg | 30 mg |
|---|---|---|---|---|---|---|
| | 2 mg Thio. | Thio. | Thio. | Thio. | Thio. | Thio. |
| 50 mg Li | 50/2 | 50/5 | 50/15 | 50/20 | 50/25 | 50/30 |
| 300 mg Li | 300/2 | 300/5 | 300/15 | 300/20 | 300/25 | 300/30 |
| 600 mg Li | 600/2 | 600/5 | 600/15 | 600/20 | 600/25 | 600/60 |
| 750 mg Li | 750/2 | 750/5 | 750/15 | 750/20 | 600/25 | 750/30 |
| 900 mg Li | 900/2 | 900/5 | 900/15 | 900/20 | 900/25 | 900/30 |
| 1200 mg Li | 1200/2 | 1200/5 | 1200/15 | 1200/20 | 1200/25 | 1200/30 |
| 1500 mg Li | 1500/2 | 1500/5 | 1500/15 | 1500/20 | 1500/25 | 1500/30 |

Table 18 provides an example of particular dosages of loxapine succinate combined with lithium in single formulations. In particular embodiments, the following non-limiting dosages of loxapine succinate can be combined with lithium: 10, 20, 40, 60, 80, 100 mg.

TABLE 18
Exemplary Dosages of Loxapine Succinate Combined with
Exemplary Dosages of Lithium (Dosages in bold print indicate maintenance doses)

| | | 20 mg | 40 mg | 60 mg | 80 mg | 100 mg |
|---|---|---|---|---|---|---|
| | 10 mg Lox. | Lox. | Lox. | Lox. | Lox. | Lox. |
| 50 mg Li | 50/10 | 50/20 | 50/40 | 50/60 | 50/80 | 50/100 |
| 300 mg Li | 300/10 | 300/20 | 300/40 | 300/60 | 300/80 | 300/100 |
| 600 mg Li | 600/10 | 600/20 | 600/40 | 600/60 | 600/80 | 600/100 |
| 750 mg Li | 750/10 | 750/20 | 750/40 | 750/60 | 750/80 | 750/100 |
| 900 mg Li | 900/10 | 900/20 | 900/40 | 900/60 | 900/80 | 900/100 |
| 1200 mg Li | 1200/10 | 1200/20 | 1200/40 | 1200/60 | 1200/80 | 1200/100 |
| 1500 mg Li | 1500/10 | 1500/20 | 1500/40 | 1500/60 | 1500/80 | 1500/100 |

Table 19 provides an example of particular dosages of perphenazine combined with lithium in single formulations. In particular embodiments, the following non-limiting dosages of perphenazine can be combined with lithium: 2, 5, 10, 15, 20, 25, 30, 35, 40 mg.

TABLE 19
Exemplary Dosages of Perphenazine Combined with Exemplary
Dosages of Lithium (Dosages in bold print indicate maintenance doses)

| | 2 mg | 5 mg | 10 mg | 15 mg | 20 mg | 25 mg | 30 mg | 35 mg | 40 mg |
|---|---|---|---|---|---|---|---|---|---|
| | Per. | Per. | Per. | Per. | Per. | Per. | Per. | Per. | Per. |
| 50 mg Li | 50/2 | 50/5 | 50/10 | 50/15 | 50/20 | 50/25 | 50/30 | 50/35 | 50/40 |
| 300 mg Li | 300/2 | 300/5 | 300/10 | 300/15 | 300/20 | 300/25 | 300/30 | 300/35 | 300/40 |
| 600 mg Li | 600/2 | 600/5 | 600/10 | 600/15 | 600/20 | 600/25 | 600/30 | 600/35 | 600/40 |
| 750 mg Li | 750/2 | 750/5 | 750/10 | 750/15 | 750/20 | 750/25 | 750/30 | 750/35 | 750/40 |
| 900 mg Li | 900/2 | 900/5 | 900/10 | 900/15 | 900/20 | 900/25 | 900/30 | 900/35 | 900/40 |
| 1200 mg Li | 1200/2 | 1200/5 | 1200/10 | 1200/15 | 1200/20 | 1200/25 | 1200/30 | 1200/35 | 1200/40 |
| 1500 mg Li | 1500/2 | 1500/5 | 1500/10 | 1500/15 | 1500/20 | 1500/25 | 1500/30 | 1500/35 | 1500/40 |

Table 20 provides an example of particular dosages of molindone hydrochloride combined with lithium in single formulations. In particular embodiments, the following non-limiting dosages of molindone hydrochloride can be combined with lithium: 10, 50, 75, 100 mg.

TABLE 20
Exemplary Dosages of Molindone Hydrochloride Combined with
Exemplary Dosages of Lithium (Dosages in bold print indicate
maintenance doses)

| | 10 mg Mol. | 50 mg Mol. | 75 mg Mol. | 100 mg Mol. |
|---|---|---|---|---|
| 50 mg Li | 50/10 | 50/50 | 50/75 | 50/100 |
| 300 mg Li | 300/10 | 300/50 | 300/75 | 300/100 |
| 600 mg Li | 600/10 | 600/50 | 600/75 | 600/100 |
| 750 mg Li | 750/10 | 750/50 | 750/75 | 750/100 |
| 900 mg Li | 750/10 | 900/50 | 900/75 | 900/100 |
| 1200 mg Li | 1200/10 | 1200/50 | 1200/75 | 1200/100 |
| 1500 mg Li | 1200/10 | 1200/50 | 1500/75 | 1500/100 |

### E. Lithium+SSRI+Tricyclic Antidepressant

In other embodiments described herein, the third component that can be combined with lithium and an SSRI are tricyclic antidepressants for the treatment of mood spectrum disorder. In particular embodiments, the tricyclic antidepressants can be combined with lithium and an SSRI into a single formulation. In yet further embodiments, the tricyclic antidepressants can be co-administered with the lithium/SSRI combination. In another embodiment described herein the tricyclic antidepressants can be combined with lithium alone without an SSRI.

Table 21 provides non-limiting examples of two tricyclic antidepressants that can be combined as a third component with lithium and an SSRI or with lithium alone. The table provides typical dosage ranges for the tricyclic antidepressants.

TABLE 21
Examples of Tricyclic Antidepressants and Dosage Ranges

| Tricyclic Antidepressants | Dosage Ranges |
|---|---|
| Desipramine | 25-300 mg/day |
| Nortriptyline | 25-150 mg/day |

In embodiments described herein, any of the disclosed or described combinations of lithium with an SSRI can be combined with a tricyclic antidepressant. In particular embodiments, any of the disclosed or described combinations of lithium with an SSRI can be combined with a tricyclic antidepressant in the following exemplary non-limiting dosages: (1) desipramine: 25, 50, 100, 150, 200, 250, 300 mg; and (2) Nortriptyline: 25, 50, 75, 100, 125, and 150 mg. These non-limiting dosages may be combined in either a single formulation with lithium and an SSRI or co-administered with the combination of lithium and an SSRI.

Table 22 provides an example of particular dosages of SERATROL, combined with or co-administered with exemplary dosages of desipramine. In further embodiments, other dosages of SERATROL may be combined with or co-administered with dosages of Nortriptyline as disclosed herein.

TABLE 22
Example of Dosages of Desipramine Combined or Co-
Administered with Some Exemplary Dosages of SERATROL

| | 25 mg Des. | 50 mg Des. | 100 mg Des. | 200 mg Des. | 300 mg Des. |
|---|---|---|---|---|---|
| 150/10 SERATROL | 150/10/25 | 150/10/50 | 150/10/100 | 150/10/200 | 150/10/300 |
| 300/20 SERATROL | 300/20/25 | 300/20/50 | 300/20/100 | 300/20/200 | 300/20/300 |
| 450/30 SERATROL | 450/30/25 | 450/30/50 | 450/30/100 | 450/30/200 | 450/30/300 |
| 600/40 SERATROL | 600/400/25 | 600/40/50 | 600/40/100 | 600/40/200 | 600/40/300 |
| 750/50 SERATROL | 750/50/25 | 750/50/50 | 750/50/100 | 750/50/200 | 750/50/300 |

In yet further embodiments, tricyclic antidepressants can be combined with lithium into single formulation dosages. The combination of lithium and nortriptyline or desipramine is a powerful and very inexpensive mood stabilizing agent for treating mood spectrum disorder.

Table 23 provides non-limiting examples of particular embodiments combining lithium with nortriptyline. Other embodiments include combining desipramine with lithium.

TABLE 23
Exemplary of Dosages of Nortriptyline Combined with Exemplary
Dosages of Lithium

| | 25 mg No. | 50 mg No. | 75 mg No. | 100 mg No. | 125 mg No. | 150 mg No. |
|---|---|---|---|---|---|---|
| 150 mg Li | 150/25 | 150/50 | 150/75 | 150/100 | 150/125 | 150/150 |
| 300 mg Li | 300/25 | 300/50 | 300/75 | 300/100 | 300/125 | 300/150 |
| 450 mg Li | 450/25 | 450/50 | 450/75 | 450/100 | 450/125 | 450/150 |
| 600 mg Li | 600/25 | 600/50 | 600/75 | 600/100 | 600/125 | 600/150 |
| 750 mg Li | 750/25 | 750/50 | 750/75 | 750/100 | 750/125 | 750/150 |
| 900 mg Li | 900/25 | 900/50 | 900/75 | 900/100 | 900/125 | 900/150 |
| 1050 mg Li | 1050/25 | 1050/50 | 1050/75 | 1050/100 | 1050/125 | 1050/150 |
| 1200 mg Li | 1200/25 | 1200/50 | 1200/75 | 1200/100 | 1200/125 | 1200/150 |

### F. Lithium+MAOIs

In additional embodiments, monoamine oxidase inhibitors can be combined with lithium in a single formulation for the treatment of mood spectrum disorder. Three particular embodiments include phenelzine, tranylcypromine, and selegiline as the MAOI's combined with lithium. In certain embodiments, the combination of lithium and an MAOI may be effective in treating Bipolar I Disorder, depressed, manic, and mixed types, in both acute and maintenance phases, Bipolar II Disorder, Borderline Personality Disorder, Cyclothymia, Depressive Personality Disorder, Dysthymia, Substance Use Disorder, and Treatment Resistant Depression, in both acute and maintenance phases. In a non-limiting example, a lithium and selegiline combination is effective in treating a patient with suicidal depression whose condition was previously worsened by treatment with an SSRI.

Table 24 provides a non-limiting range of dosages for phenelzine sulfate, tranylcypromine sulfate, and selegiline in oral and patch formulations.

TABLE 24
Examples of Monoamine Oxidase Inhibitors and Dosage Ranges

| Monoamine Oxidase Inhibitors | Dosage Ranges |
|---|---|
| Phenelzine Sulfate | 15-90 mg/day |
| Tranylcypromine Sulfate | 10-30 mg/day |
| Selegiline oral | 1-20 mg |
| Selegiline patch | 6-12 mg |

Table 25 provides an example of particular dosages of phenelzine combined with lithium in single formulations. In further embodiments, tranylcypromine is combined with lithium in single formulations. In particular embodiments, the following non-limiting dosages of MAOI's can be combined with lithium in a single formulation: (1) phenelzine sulfate: 15, 30, 45, 60, 75, and 90 mg; (2) tranylcypromine sulfate: 10, 20, 30 mg; (3) selegiline oral: 1, 5, 10, 15, 20 mg; and (4) selegiline patch: 6, 9, 12 mg.

TABLE 25
Exemplary Dosages of Phenelzine Sulfate Combined with
Exemplary Dosages of Lithium

| | 15 mg Ph. | 30 mg Ph. | 45 mg Ph. | 60 mg Ph. | 75 mg Ph. | 90 mg Ph. |
|---|---|---|---|---|---|---|
| 150 mg Li | 150/15 | 150/30 | 150/45 | 150/60 | 150/75 | 150/90 |
| 300 mg Li | 300/15 | 300/30 | 300/45 | 300/60 | 300/75 | 300/90 |
| 450 mg Li | 450/15 | 450/30 | 450/45 | 450/60 | 450/75 | 450/90 |
| 600 mg Li | 600/15 | 600/30 | 600/45 | 600/60 | 600/75 | 600/90 |
| 750 mg Li | 750/15 | 750/30 | 750/45 | 750/60 | 750/75 | 750/90 |
| 900 mg Li | 900/15 | 900/30 | 900/45 | 900/60 | 900/75 | 900/90 |
| 1050 mg Li | 1050/15 | 1050/30 | 1050/45 | 1050/60 | 1050/75 | 1050/90 |
| 1200 mg Li | 1200/15 | 1200/30 | 1200/45 | 1200/60 | 1200/75 | 1200/90 |

Table 26 provides a nonlimiting example of particular dosages of selegiline in the oral form in combination with lithium in single formulation.

TABLE 26
Exemplary Dosages of Oral Selegiline Combined with Exemplary
Dosages of Lithium

| | 1 mg Sel. | 5 mg Sel. | 10 mg Sel. | 15 mg Sel. | 20 mg Sel. |
|---|---|---|---|---|---|
| 50 mg Li | 50/1 | 50/5 | 50/10 | 50/15 | 50/20 |
| 300 mg Li | 300/1 | 300/5 | 300/10 | 300/15 | 300/20 |
| 600 mg Li | 600/1 | 600/5 | 600/10 | 600/15 | 600/20 |
| 750 mg Li | 750/1 | 750/5 | 750/10 | 750/15 | 750/20 |
| 900 mg Li | 900/1 | 900/5 | 900/10 | 900/15 | 900/20 |

Table 27 provides a nonlimiting example of particular dosages of selegiline in the patch form in combination with lithium in single formulation.

TABLE 27
Exemplary Dosages of Patch Selegiline Combined with Exemplary
Dosages of Lithium (Dosages in bold print indicate maintenance doses)

| | 6 mg Sel. | 9 mg Sel. | 12 mg Sel. |
|---|---|---|---|
| 50 mg Li | 50/6 | 50/9 | 50/12 |
| 300 mg Li | 300/6 | 300/9 | 300/12 |
| 600 mg Li | 600/6 | 600/9 | 600/12 |
| 750 mg Li | 750/6 | 750/9 | 750/12 |
| 900 mg Li | 900/6 | 900/9 | 900/12 |
| 1200 mg Li | 1200/6 | 1200/9 | 1200/12 |
| 1500 mg Li | 1500/6 | 1500/9 | 1500/12 |

### G. Lithium+Atypical Antidepressant

In additional embodiments, lithium may be combined with atypical antidepressants in a single formulation for the treatment of mood spectrum disorder. Two particular embodiments include nefazodone and trazodone as the atypical antidepressants to be combined with lithium.

Table 28 provides a non-limiting range of dosages for nefazodone and trazodone.

TABLE 28
Examples of Atypical Antidepressants and Dosage Ranges

| Atypical Antidepressants | Dosage Ranges |
|---|---|
| Nefazodone | 50-600 mg |
| Trazodone | 25-600 mg |

Table 29 provides a nonlimiting example of particular dosages of nefazodone in combination with lithium in single formulation. In particular embodiments, the following non-limiting dosages of nefazodone can be combined with lithium: 50, 100, 200, 300, 400, 500, 600 mg.

TABLE 29
Exemplary Dosages of Nefazodone Combined with Exemplary
Dosages of Lithium (Dosages in bold print indicate maintenance doses)

| | 50 mg | 100 mg | 200 mg | 300 mg | 400 mg | 500 mg | 600 mg |
|---|---|---|---|---|---|---|---|
| | Nef. | Nef. | Nef. | Nef. | Nef. | Nef. | Nef. |
| 50 mg Li | 50/50 | 50/100 | 50/200 | 50/300 | 50/400 | 50/500 | 50/600 |
| 300 mg Li | 300/50 | 300/100 | 300/200 | 300/300 | 300/400 | 300/500 | 300/600 |
| 600 mg Li | 600/50 | 600/100 | 600/200 | 600/300 | 600/400 | 600/500 | 600/600 |
| 750 mg Li | 750/50 | 750/100 | 750/200 | 750/300 | 750/400 | 750/500 | 750/600 |
| 900 mg Li | 900/50 | 900/100 | 900/200 | 900/300 | 900/400 | 900/500 | 900/600 |
| 1200 mg Li | 1200/50 | 1200/100 | 1200/200 | 1200/300 | 1200/400 | 1200/500 | 1200/600 |
| 1500 mg Li | 1500/50 | 1500/100 | 1500/200 | 1500/300 | 1500/400 | 1500/500 | 1500/600 |

Table 30 provides a nonlimiting example of particular dosages of trazodone in combination with lithium in single formulation. In particular embodiments, the following non-limiting dosages of trazodone can be combined with lithium: 25, 100, 200, 300, 400, 500, 600 mg.

TABLE 30
Exemplary Dosages of Trazodone Combined with Exemplary
Dosages of Lithium (Dosages in bold print indicate maintenance doses)

| | 25 mg | 100 mg | 200 mg | 300 mg | 400 mg | 500 mg | 600 mg |
|---|---|---|---|---|---|---|---|
| | Traz. | Traz. | Traz. | Traz. | Traz. | Traz. | Traz. |
| 50 mg Li | 50/25 | 50/100 | 50/200 | 50/300 | 50/400 | 50/500 | 50/600 |
| 300 mg Li | 300/25 | 300/100 | 300/200 | 300/300 | 300/400 | 300/500 | 300/600 |
| 600 mg Li | 600/25 | 600/100 | 600/200 | 600/300 | 600/400 | 600/500 | 600/600 |
| 750 mg Li | 750/25 | 750/100 | 750/200 | 750/300 | 750/400 | 750/500 | 750/600 |
| 900 mg Li | 900/25 | 900/100 | 900/200 | 900/300 | 900/400 | 900/500 | 900/600 |
| 1200 mg Li | 1200/25 | 1200/100 | 1200/200 | 1200/300 | 1200/400 | 1200/500 | 1200/600 |
| 1500 mg Li | 1500/25 | 1500/100 | 1500/200 | 1500/300 | 1500/400 | 1500/500 | 1500/600 |

In yet further embodiments, a third component that may be combined with lithium and an SSRI is bupropion hydrochloride or mirtazapine. In particular embodiments, the bupropion or mirtazapine can be combined with lithium and an SSRI into a single formulation. In other embodiments, bupropion or mirtazapine can be co-administered with lithium and an SSRI. In yet another embodiment, lithium may be combined with bupropion or mirtazapine into a single formulation.

Table 31 provides an example of particular dosages of SERATROL combined with or co-administered with exemplary dosages of bupropion hydrochloride extended release. Appropriate exemplary dosages of mirtazapine include, but are not limited by, 7.5, 15, 30, 45, and 60 mg. These particular examples are in no way limiting, other lithium/SSRI combinations may also be combined with bupropion or mirtazapine.

TABLE 31
Example of Dosages of Bupropion Hydrochloride Extended
Release Combined or Co-Administered with Some Exemplary Dosages of
SERATROL

| | | | 450 mg Bup. |
|---|---|---|---|
| | 150 mg Bup. HCl | 300 mg Bup. HCl | HCl |
| 150/10 SERATROL | 150/10/150 | 150/10/300 | 150/10/450 |
| 300/20 SERATROL | 300/20/150 | 300/20/300 | 300/20/450 |
| 450/30 SERATOL | 450/30/150 | 450/30/300 | 450/20/450 |
| 600/40 SERATROL | 600/40/150 | 600/40/300 | 600/40/450 |
| 750/50 SERATROL | 750/50/150 | 750/50/300 | 750/50/450 |

In other embodiments, bupropion or mirtazapine can also be combined into a single formulation with lithium. Table 32 provides non-limiting examples of particular embodiments combining lithium with mirtazapine.

TABLE 32
Exemplary Dosages of Mirtazapine Combined with Exemplary
Dosages of Lithium

| | 15 mg | 30 mg | 45 mg |
|---|---|---|---|
| | Mirtazapine | Mirtazapine | Mirtazapine |
| 150 mg Li | 150/15 | 150/30 | 150/450 |
| 300 mg Li | 300/15 | 300/30 | 300/45 |
| 450 mg Li | 450/15 | 450/30 | 450/45 |
| 600 mg Li | 600/15 | 600/30 | 600/45 |
| 750 mg Li | 750/15 | 750/30 | 750/45 |
| 900 mg Li | 900/15 | 900/30 | 900/45 |
| 1050 mg Li | 1050/15 | 1050/30 | 1050/45 |

### I. Bupropion+Tricyclic Antidepressant

In other embodiments, bupropion can be combined with a tricyclic antidepressant in a single formulation for the treatment of mood spectrum disorder. Particular embodiments of the bupropion and tricyclic antidepressant combination are effective for treating Bipolar I Disorder, depressed and mixed types, in both acute and maintenance phases, Borderline Personality Disorder, Cyclothymia, Dysthymia, Depressive Personality Disorder, Substance Use Disorder, and Treatment Resistant Depression in both acute and maintenance phases. In a particular embodiment, where the tricyclic antidepressant is desipramine, the combination is effective for treatment of Treatment Resistant Depression after trials with SSRIs had failed.

Table 33 provides non-limiting examples of tricyclic antidepressants and typical dosage ranges that can be combined with bupropion.

TABLE 33
Examples of Tricyclic Antidepressants and Dosage Ranges

| Tricyclic Antidepressants | Dosage Ranges |
|---|---|
| Amitriptyline | 25-300 mg |
| Clomipramine | 25-250 mg |
| Doxepin | 25-300 mg |
| Imipramine | 25-300 mg |
| Trimipramine | 25-600 mg |
| Desipramine | 25-300 mg |
| Nortriptyline | 25-250 mg |
| Protriptyline | 5-60 mg |

Table 34 provides an example of particular dosages of amitriptyline combined with bupropion in single formulations. In particular embodiments, the following non-limiting dosages of amitriptyline can be combined with bupropion: 25, 100, 200, 300 mg.

TABLE 34
Exemplary Dosages of Amitriptyline Combined with Exemplary
Dosages of Bupropion (Dosages in bold print indicate maintenance doses)

| | 25 mg Am. | 100 mg Am. | 200 mg Am. | 300 mg Am. |
|---|---|---|---|---|
| 25 mg Bup. | 25/25 | 25/100 | 25/200 | 25/300 |
| 75 mg Bup. | 75/25 | 75/100 | 75/200 | 75/300 |
| 100 mg Bup. | 100/25 | 100/100 | 100/200 | 100/300 |
| 150 mg Bup. | 150/25 | 150/100 | 150/200 | 150/300 |
| 300 mg Bup. | 300/25 | 300/100 | 300/200 | 300/300 |
| 450 mg Bup. | 450/25 | 450/100 | 450/200 | 450/300 |

Table 35 provides an example of particular dosages of clomipramine combined with bupropion in single formulations. In particular embodiments, the following non-limiting dosages of clomipramine can be combined with bupropion: 25, 50, 100, 150, 200, 250 mg.

TABLE 35
Exemplary Dosages of Clomipramine Combined with Exemplary
Dosages of Bupropion (Dosages in bold print indicate maintenance doses)

| | 25 mg | 50 mg | 100 mg | 150 mg | 200 mg | 250 mg |
|---|---|---|---|---|---|---|
| | Clo. | Clo. | Clo. | Clo. | Clo. | Clo. |
| 25 mg Bup. | 25/25 | 25/50 | 25/100 | 25/150 | 25/200 | 25/250 |
| 75 mg Bup. | 75/25 | 75/50 | 75/100 | 75/150 | 75/200 | 75/250 |
| 100 mg Bup. | 100/25 | 100/50 | 100/100 | 100/150 | 100/200 | 100/250 |
| 150 mg Bup. | 150/25 | 150/50 | 150/100 | 150/150 | 150/200 | 150/250 |
| 300 mg Bup. | 300/25 | 300/50 | 300/100 | 300/150 | 300/200 | 300/250 |
| 450 mg Bup. | 450/25 | 450/50 | 450/100 | 450/150 | 450/200 | 450/250 |

Table 36 provides. an example of particular dosages of doxepin combined with bupropion in single formulations. In additional embodiments, the following non-limiting dosages of doxepin can be combined with bupropion: 25, 100, 200, 300 mg.

TABLE 36
Exemplary Dosages of Doxepin Combined with Exemplary
Dosages of Bupropion (Dosages in bold print indicate maintenance doses)

| | 25 mg Dox. | 100 mg Dox. | 200 mg Dox. | 300 mg Dox. |
|---|---|---|---|---|
| 25 mg Bup. | 25/25 | 25/100 | 25/200 | 25/300 |
| 75 mg Bup. | 75/25 | 75/100 | 75/200 | 75/300 |
| 100 mg Bup. | 100/25 | 100/100 | 100/200 | 100/300 |
| 150 mg Bup. | 150/25 | 150/100 | 150/200 | 150/300 |
| 300 mg Bup. | 300/25 | 300/100 | 300/200 | 300/300 |

Table 37 provides an example of particular dosages of imipramine combined with bupropion in single formulations. In additional embodiments, the following non-limiting dosages of imipramine can be combined with bupropion: 25, 100, 200, 300 mg.

TABLE 37
Exemplary Dosages of Imipramine Combined with Exemplary
Dosages of Bupropion (Dosages in bold print indicate maintenance doses)

| | 25 mg Im. | 100 mg Im. | 200 mg Im. | 300 mg Im. |
|---|---|---|---|---|
| 25 mg Bup. | 25/25 | 25/100 | 25/200 | 25/300 |
| 75 mg Bup. | 75/25 | 75/100 | 75/200 | 75/300 |
| 100 mg Bup. | 100/25 | 100/100 | 100/200 | 100/300 |
| 150 mg Bup. | 150/25 | 150/100 | 150/200 | 150/300 |
| 300 mg Bup. | 300/25 | 300/100 | 300/200 | 300/300 |
| 450 mg Bup. | 450/25 | 450/100 | 450/200 | 450/300 |

Table 38 provides an example of particular dosages of trimipramine combined with bupropion in single formulations. In additional embodiments, the following non-limiting dosages of trimipramine can be combined with bupropion: 25, 100, 200, 300, 400, 500, 600 mg.

TABLE 38
Exemplary Dosages of Trimipramine Combined with Exemplary
Dosages of Bupropion

| | | 100 mg | 200 mg | 300 mg | 400 mg | | |
|---|---|---|---|---|---|---|---|
| | 25 mg Tri. | Tri. | Tri. | Tri. | Tri. | 500 mg Tri. | 600 mg Tri. |
| 25 mg | 25/25 | 25/100 | 25/200 | 25/300 | 25/400 | 25/500 | 25/600 |
| Bup. | | | | | | | |
| 75 mg | 75/25 | 75/100 | 75/200 | 75/300 | 75/400 | 75/500 | 75/600 |
| Bup. | | | | | | | |
| 100 mg | 100/25 | 100/100 | 100/200 | 100/300 | 100/400 | 100/500 | 100/600 |
| Bup. | | | | | | | |
| 150 mg | 150/25 | 150/100 | 150/200 | 150/300 | 150/400 | 150/500 | 150/600 |
| Bup. | | | | | | | |
| 300 mg | 300/25 | 300/100 | 300/200 | 300/300 | 300/400 | 300/500 | 300/600 |
| Bup. | | | | | | | |
| 450 mg | 450/25 | 450/100 | 450/200 | 450/300 | 450/400 | 450/500 | 450/600 |
| Bup. | | | | | | | |

Table 39 provides an example of particular dosages of desipramine combined with bupropion in single formulations. In particular embodiments, the following non-limiting dosages of desipramine can be combined with bupropion: 25, 100, 200, 300 mg.

TABLE 39
Exemplary Dosages of Desipramine Combined with Exemplary
Dosages of Bupropion (Dosages in bold print indicate maintenance doses)

| | 25 mg Des. | 100 mg Des. | 200 mg Des. | 300 mg Des. |
|---|---|---|---|---|
| 25 mg Bup. | 25/25 | 25/100 | 25/200 | 25/300 |
| 75 mg Bup. | 75/25 | 75/100 | 75/200 | 75/300 |
| 100 mg Bup. | 100/25 | 100/100 | 100/200 | 100/300 |
| 150 mg Bup. | 150/25 | 150/100 | 150/200 | 150/300 |
| 300 mg Bup. | 300/25 | 300/100 | 300/200 | 300/300 |
| 450 mg Bup. | 450/25 | 450/100 | 450/200 | 450/300 |

Table 40 provides an example of particular dosages of nortriptyline combined with bupropion in single formulations. In particular embodiments, the following non-limiting dosages of nortriptyline can be combined with bupropion: 25, 50, 100, 200, 250 mg.

TABLE 40
Exemplary Dosages of Nortriptyline Combined
with Exemplary Dosages of Bupropion
(Dosages in bold print indicate maintenance doses)

| | 25 mg | 50 mg | 100 mg | 150 mg | 200 mg | 250 mg |
|---|---|---|---|---|---|---|
| | Nor. | Nor. | Nor. | Nor. | Nor. | Nor. |
| 25 mg Bup. | 25/25 | 25/50 | 25/100 | 25/150 | 25/200 | 25/250 |
| 75 mg Bup. | 75/25 | 75/50 | 75/100 | 75/150 | 75/200 | 75/250 |
| 100 mg Bup. | 100/25 | 100/50 | 100/100 | 100/150 | 100/200 | 100/250 |
| 150 mg Bup. | 150/25 | 150/50 | 150/100 | 150/150 | 150/200 | 150/250 |
| 300 mg Bup. | 300/25 | 300/50 | 300/100 | 300/150 | 300/200 | 300/250 |
| 450 mg Bup. | 450/25 | 450/50 | 450/100 | 450/150 | 450/200 | 450/250 |

Table 41 provides an example of particular dosages of protriptyline combined with bupropion in single formulations. In further embodiments, the following non-limiting dosages of protriptyline can be combined with bupropion: 5, 30, 45, 60 mg.

TABLE 41
Exemplary Dosages of Protriptyline Combined with Exemplary
Dosages of Bupropion (Dosages in bold print indicate maintenance doses)

| | 5 mg Pro. | 30 mg Pro. | 45 mg Pro. | 60 mg Pro. |
|---|---|---|---|---|
| 25 mg Bup. | 25/5 | 25/30 | 25/45 | 25/60 |
| 75 mg Bup. | 75/5 | 75/30 | 75/45 | 75/60 |
| 100 mg Bup. | 100/5 | 100/30 | 100/45 | 100/60 |
| 150 mg Bup. | 150/5 | 150/30 | 150/45 | 150/60 |
| 300 mg Bup. | 300/5 | 300/30 | 300/45 | 300/60 |
| 450 mg Bup. | 450/5 | 450/30 | 450/45 | 450/60 |

### J. Bupropion+Tetracyclic Antidepressant

In other embodiments, bupropion may be combined with a tetracyclic antidepressant in a single formulation for the treatment of mood spectrum disorder. In certain embodiments, the combination of bupropion and a tetracyclic antidepressant is effective for the treatment of Bipolar I Disorder, depressed and mixed types, in both acute and maintenance phases, Borderline Personality Disorder, Cyclothymia, Dysthymia, Depressive Personality Disorder, Substance Use Disorder, and Treatment Resistant Depression in both acute and maintenance phases.

Table 42 provides non-limiting examples of tetracyclic antidepressants and typical dosage ranges that can be combined with bupropion.

TABLE 42
Examples of Tetracyclic Antidepressants and Dosage Ranges

| Tetracyclic Antidepressants | Dosage Ranges |
|---|---|
| Amoxapine | 25-600 mg |
| Maprotiline | 25-225 mg |

Table 43 provides an example of particular dosages of amoxapine combined with bupropion in single formulations. In particular embodiments, the following non-limiting dosages of amoxapine can be combined with bupropion: 25, 100, 200, 300, 400, 500, 600 mg.

TABLE 43
Exemplary Dosages of Amoxapine Combined
with Exemplary Dosages of Bupropion
(Dosages in bold print indicate maintenance doses)

| | 25 mg | 100 mg | 200 mg | 300 mg | 400 mg | 500 mg | 600 mg |
|---|---|---|---|---|---|---|---|
| | Am. | Am. | Am. | Am. | Am. | Am. | Am. |
| 25 mg Bup. | 25/25 | 25/100 | 25/200 | 25/300 | 25/400 | 25/500 | 25/600 |
| 75 mg Bup. | 75/25 | 75/100 | 75/200 | 75/300 | 75/400 | 75/500 | 75/600 |
| 100 mg Bup. | 100/25 | 100/100 | 100/200 | 100/300 | 100/400 | 100/500 | 100/600 |
| 150 mg Bup. | 150/25 | 150/100 | 150/200 | 150/300 | 150/400 | 150/500 | 150/600 |
| 300 mg Bup. | 300/25 | 300/100 | 300/200 | 300/300 | 300/400 | 300/500 | 300/600 |
| 450 mg Bup. | 450/25 | 450/100 | 450/200 | 450/300 | 450/400 | 450/500 | 450/600 |

Table 44 provides an example of particular dosages of maprotiline combined with bupropion in single formulations. In particular embodiments, the following non-limiting dosages of maprotiline can be combined with bupropion: 25, 75, 125, 225 mg.

TABLE 44
Exemplary Dosages of Maprotiline Combined
with Exemplary Dosages of Bupropion
(Dosages in bold print indicate maintenance doses)

| | 25 mg | 75 mg | 125 mg | 175 mg | |
|---|---|---|---|---|---|
| | Map. | Map. | Map. | Map. | 225 mg Map. |
| 25 mg Bup. | 25/25 | 25/75 | 25/125 | 25/175 | 25/225 |
| 75 mg Bup. | 75/25 | 75/75 | 75/125 | 75/175 | 75/225 |
| 100 mg Bup. | 100/25 | 100/75 | 100/125 | 100/175 | 100/225 |
| 150 mg Bup. | 150/25 | 150/75 | 150/125 | 150/175 | 150/225 |
| 300 mg Bup. | 300/25 | 300/75 | 300/125 | 300/175 | 300/225 |
| 450 mg Bup. | 450/25 | 450/75 | 450/125 | 450/175 | 450/225 |

### K. Bupropion+Antiseizure

In certain embodiments, bupropion may be combined with divalproex sodium for the treatment of mood spectrum disorder in a single formulation. Particular embodiments of the combination of bupropion and divalproex sodium are effective in treating Bipolar I Disorder, manic and mixed types, in both acute and maintenance phases and Schizoaffective Disorder, in both acute and maintenance phases.

Table 45 provides non-limiting examples of particular embodiments combining bupropion with divalproex sodium.

TABLE 45
Exemplary Dosages of Divalproex Sodium Combined with
Exemplary Dosages of Bupropion

| | 250 mg | 500 mg | 750 mg | 1000 mg | 1250 mg | 1500 mg | 1750 mg |
|---|---|---|---|---|---|---|---|
| | Div. | Div. | Div. | Div. | Div. | Div. | Div. |
| 25 mg Bup. | 25/250 | 25/500 | — | — | — | — | — |
| 75 mg Bup. | — | — | — | — | — | — | — |
| 100 mg | — | — | 100/750 | 100/1000 | 100/1250 | — | — |
| Bup. | | | | | | | |
| 150 mg | — | — | 150/750 | 150/1000 | 150/1250 | 150/1500 | 150/1750 |
| Bup. | | | | | | | |
| 300 mg | — | — | 300/750 | 300/1000 | 300/1250 | 300/1500 | 300/1750 |
| Bup. | | | | | | | |
| 450 mg | — | — | — | 450/1000 | 450/1250 | 450/1500 | 450/1750 |
| Bup. | | | | | | | |

### L. Lithium+Bupropion+Lamotrigine

In certain embodiments, lithium may be combined with bupropion and lamotrigine for the treatment of mood spectrum disorder in a single formulation. Particular embodiments of the combination of lithium, bupropion, and lamotrigine are effective in treating Bipolar I Disorder, depressed, manic, and mixed types, in both acute and maintenance phases, Borderline Personality Disorder, Cyclothymia, Dysthymia, Depressive Personality Disorder, Substance Use Disorder, and Treatment Resistant Depression in both acute and maintenance phases. In an embodiment lithium and bupropion may be combined with a third component into a single formulation. In a another embodiment, lithium and lamotrigine may be combined with a third component. In a particular embodiment, lithium is combined with bupropion and lamotrigene in a single formulation. Table 46A provides a non-limiting example of particular dosages of lithium and bupropion with lamotrigine in combination in a single formulation. Table 46B provides a non-limiting example of particular dosages of lithium and lamotrigene with bupropion.

In certain embodiments, the following non-limiting dosages of bupropion can be combined with lithium and lamotrigine: 25, 75, 150, 200, 250, and 300 mg. In other embodiments, the following non-limiting dosages of bupropion can be combined with lithium and lamotrigine: 150, 300 and 450 mg. In yet further embodiments, the following non-limiting dosages of lamotrigine can be combined with lithium and bupropion: 25, 50, 100, 150, 200, 250, and 300 mg. In other embodiments, bupropion can be combined with lamotrigine in a single formulation without lithium.

TABLE 46A
Exemplary Dosages of Lamotrigene combined with exemplary
dosages of Lithium and Bupropion in Combination

| | 25 mg | 50 mg | 100 mg | 200 mg | 300 mg |
|---|---|---|---|---|---|
| | Lamotrigene | Lamotrigene | Lamotrigene | Lamotrigene | Lamotrigene |
| 150 mg/150 mg | 150/150/25 | 150/150/50 | 150/150/100 | 150/150/200 | 150/150/300 |
| Li/Bupropion | | | | | |
| 150 mg/300 mg | 150/300/25 | 150/300/50 | 150/300/100 | 150/300/200 | 150/300/300 |
| Li/Bupropion | | | | | |
| 150 mg/450 mg | 150/450/25 | 150/450/50 | 150/450/100 | 150/450/200 | 150/450/300 |
| Li/Bupropion | | | | | |
| 300 mg/150 mg | 300/150/25 | 300/150/50 | 300/150/100 | 300/150/200 | 300/150/300 |
| Li/Bupropion | | | | | |
| 300 mg/300 mg | 300/300/25 | 300/300/50 | 300/300/100 | 300/300/200 | 300/300/300 |
| Li/Bupropion | | | | | |
| 300 mg/450 mg | 300/450/25 | 300/450/50 | 300/450/100 | 300/450/200 | 300/450/300 |
| Li/Bupropion | | | | | |
| 450 mg/150 mg | 450/150/25 | 450/150/50 | 450/150/100 | 450/150/200 | 450/150/300 |
| Li/Bupropion | | | | | |
| 450 mg/300 mg | 450/300/25 | 450/300/50 | 450/300/100 | 450/300/200 | 450/300/300 |
| Li/Bupropion | | | | | |
| 450 mg/450 mg | 450/450/25 | 450/450/50 | 450/450/100 | 450/450/200 | 450/450/300 |
| Li/Bupropion | | | | | |
| 600 mg/150 mg | 600/150/25 | 600/150/50 | 600/150/100 | 600/150/200 | 600/150/300 |
| Li/Bupropion | | | | | |
| 600 mg/300 mg | 600/300/25 | 600/300/50 | 600/300/100 | 600/300/200 | 600/300/300 |
| Li/Bupropion | | | | | |
| 600 mg/450 mg | 600/450/25 | 600/450/50 | 600/450/100 | 600/450/200 | 600/450/300 |
| Li/Bupropion | | | | | |
| 900 mg/150 mg | 900/150/25 | 900/150/50 | 900/150/100 | 900/150/200 | 900/150/300 |
| Li/Bupropion | | | | | |
| 900 mg/300 mg | 900/300/25 | 900/300/50 | 900/300/100 | 900/300/200 | 900/300/300 |
| Li/Bupropion | | | | | |
| 900 mg/450 mg | 900/450/25 | 900/450/50 | 900/450/100 | 900/450/200 | 900/450/300 |
| Li/Bupropion | | | | | |

TABLE 46B
Exemplary Dosages of Bupropion combined with exemplary
dosages of Lamotrigine and Lithium in Combination

| | 25 mg | 75 mg | 150 mg | 200 mg | 250 mg | 300 mg |
|---|---|---|---|---|---|---|
| | Bup. | Bup. | Bup. | Bup. | Bup. | Bup. |
| 50 mg/25 mg | 50/25/25 | 50/25/75 | 50/25/150 | 50/25/200 | 50/25/250 | 50/25/300 |
| Li/Lam. | | | | | | |
| 300 mg/50 mg | 300/50/25 | 300/50/75 | 300/50/150 | 300/50/200 | 300/50/250 | 300/50/300 |
| Li/Lam. | | | | | | |
| 600 mg/100 mg | 600/100/25 | 600/100/75 | 600/100/150 | 600/100/200 | 600/100/250 | 600/100/300 |
| Li/Lam. | | | | | | |
| 750 mg/150 mg | 750/150/25 | 750/150/75 | 750/150/150 | 750/150/200 | 750/150/250 | 750/150/300 |
| Li/Lam. | | | | | | |
| 900 mg/200 mg | 900/200/25 | 900/200/75 | 900/200/150 | 900/200/200 | 900/200/250 | 900/200/300 |
| Li/Lam. | | | | | | |
| 1200 mg/250 mg | 1200/250/25 | 1200/250/75 | 1200/250/150 | 1200/250/200 | 1200/250/250 | 1200/250/300 |
| Li/Lam. | | | | | | |
| 1500 mg/300 mg | 1500/300/25 | 1500/300/75 | 1500/300/150 | 1500/300/200 | 1500/300/250 | 1500/300/300 |
| Li/Lam. | | | | | | |

1. Bupropion+Lamotrigine

In other embodiments, bupropion may be combined with lamotrigine without lithium. Particular embodiments of the combination of bupropion, and lamotrigine are effective in treating Bipolar I Disorder, depressed, manic, and mixed types, in both acute and maintenance phases, Borderline Personality Disorder, Cyclothymia, Dysthymia, Depressive Personality Disorder, Substance Use Disorder, and Treatment Resistant Depression in both acute and maintenance phases.

Table 47 provides a nonlimiting example of particular dosages of bupropion in combination with particular dosages of lamotrigine in single formulation. In particular embodiments, the following non-limiting dosages of lamotrigine can be combined with bupropion: 25, 75, 150, 200, 250, 300 mg.

TABLE 47
Exemplary Dosages of Bupropion Combined
with Exemplary Dosages of Lamotrigine
(Dosage in bold print indicate maintenance doses)

| | 25 mg | 75 mg | 150 mg | 200 mg | 250 mg | 300 mg |
|---|---|---|---|---|---|---|
| | Lam. | Lam. | Lam. | Lam. | Lam. | Lam. |
| 25 mg | 25/25 | 25/75 | 25/150 | 25/200 | 25/250 | 25/300 |
| Bup. | | | | | | |
| 50 mg | 50/25 | 50/75 | 50/150 | 50/200 | 50/250 | 50/300 |
| Bup. | | | | | | |
| 100 mg | 100/25 | 100/75 | 100/150 | 100/200 | 100/250 | 100/300 |
| Bup. | | | | | | |
| 150 mg | 150/25 | 150/75 | 150/150 | 150/200 | 150/250 | 150/300 |
| Bup. | | | | | | |
| 200 mg | 200/25 | 200/75 | 200/150 | 200/200 | 200/250 | 200/300 |
| Bup. | | | | | | |
| 250 mg | 250/25 | 250/75 | 250/150 | 250/200 | 250/250 | 250/300 |
| Bup. | | | | | | |
| 300 mg | 300/25 | 300/75 | 300/150 | 300/200 | 300/250 | 300/300 |
| Bup. | | | | | | |

### M. Lithium+Bupropion+Modafinil

In certain embodiments, lithium may be combined with bupropion and modafinil for the treatment of mood spectrum disorder in a single formulation. Particular embodiments of the combination of lithium, bupropion, and modafinil are effective in treating Bipolar I Disorder, depressed and mixed types, in both acute and maintenance phases, Bipolar TI Disorder, Borderline Personality Disorder, Cyclothymia, Dysthymia, Depressive Personality Disorder, Major Depressive Disorder, Substance Use Disorder, and Treatment Resistant Depression in both acute and maintenance phases. In a non-limiting example, the lithium, bupropion, and modafinil combination may be used to treat a patient with Dysthymia and Bipolar II Disorder who has previously shown a partial response to treatment with a lithium and bupropion combination.

Table 48 provides a non-limiting example of particular dosages of modafinil combined with bupropion and lithium.

TABLE 48
Exemplary Dosages of Modafinil Combined with Exemplary
Dosages of Bupropion and Lithium

| | 200 mg Mod. | 400 mg Mod. |
|---|---|---|
| 25 mg Bup. + 300 mg Li | 25/300/200 | — |
| 150 mg Bup. + 600 mg Li | 150/600/200 | 150/600/400 |
| 300 mg Bup. + 900 mg Li | 300/900/200 | 300/900/400 |
| 450 mg Bup. + 1200 mg Li | 450/1200/200 | 450/1200/400 |

### N. Formulations

The present invention provides pharmaceutical compositions that can include at least one other agent, such as a stabilizing compound, and may be administered in any sterile, biocompatible pharmaceutical carrier, including, but not limited to, saline, buffered saline, dextrose, water, microcrystalline cellulose, hydroxypropyl methylcellulose, polysorbate 80, polyethylene glycol, magnesium stearate, carnauba wax, lactose, and/or sodium starch glycolate as known by one skilled in the art.

As is well known in the medical arts, dosages for any one patient depends upon many factors, including the patient's size, body surface area, age, the particular compound to be administered, sex, time and route of administration, general health, and interaction with other drugs being concurrently administered. Accordingly, in some embodiments of the present invention, SERATROL, can be administered to a patient alone, or in combination with other compounds disclosed herein or in pharmaceutical compositions where it is mixed with excipient(s) or other pharmaceutically acceptable carriers. In one embodiment of the present invention, the pharmaceutically acceptable carrier is pharmaceutically inert. Depending on the condition being treated and the pharmaceutical composition, these pharmaceutical compositions may be formulated and administered systemically. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, transdermal or intranasal administration. For injection, the pharmaceutical compositions described herein may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, emulsions, liposome or micellular systems, syrups, slurries, suspensions and the like, for oral or nasal ingestion by a patient to be treated.

As used herein, a “single dosage formulation” is a combined dosage formulation containing two or more active ingredients, such as, without limitation, lithium and a selective serotonin reuptake inhibitor. A single dosage formulation can be, for example and without limitation, a single tables, pill, caplet or capsule. Likewise, in the case of a liquid, gel, emulsion, liposome or micellular system, syrup, slurry, suspension and the like, the single dosage formulation contains one or more active agents, such as, without limitation, lithium and a selective serotonin reuptake inhibitor, admixed in combination in the formulation. The single dosage formulation can formulated for any safe and effective delivery route, including, for example and without limitation: oral, transmucosal, parenteral, intramuscular, subcutaneous, intramedullary, intrathecal, intraventricular, intravenous, intraperitoneal, transdermal or intranasal delivery routes.

Pharmaceutical compositions suitable for use in the present invention include compositions wherein the active ingredients are contained in an effective amount to achieve the intended purpose as disclosed herein. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions. The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethylcellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate. Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).Pharmaceutical preparations, which can be used orally, include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers. Compositions comprising a compound described herein formulated in a pharmaceutical acceptable carrier may be prepared, placed in an appropriate container, and labeled for treatment of an indicated condition.

The pharmaceutical compositions, as provided herein, may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation. Those skilled in the art will employ different formulations.

### V. Methods of Treating Patients With Mood Spectrum Disorder

Embodiments described herein include the use of a single dosage formulation containing two or more drugs, which will enable PCPs, not just psychiatrists, to treat mood spectrum disorders. In particular embodiments, the invention includes methods utilizing flexible fixed-dose drug combinations, while increasing dosages at bi-weekly intervals. These compositions and methods simplify treatment, in comparison to the thousands of drug combinations and permutations that could potentially be used to treat mood disorders during both the acute and maintenance phase treatment of patients.

For psychiatrists, this is a formidable, but possible task to successfully complete. On the other hand, PCPs are not trained to deal with this level of complexity in the treatment of psychiatric illness and, therefore, are rarely able to diagnose and treat mood disorders.

Embodiments described herein include diagnostic mood disorder screens that can be used to vary the basic drug regimen depending on the patient's symptoms and diagnosis. In embodiments of the invention, PCPs are provided with symptom severity checklists that guide them in exactly how to diagnose and prescribe the invention's combination medications. In particular embodiments, mood screens are provided in Examples 1, 2, and 3 for bipolar disorder, dysthymia and affective disorder, respectively. In yet other embodiments, the criteria from the DSM-IV and as provided herein could be used to evaluate the patient. In yet further embodiments, the mood screens will allow PCPs to quickly and effectively diagnose specific mood disorders and immediately initiate treatment with the most appropriate drug combination.

Prior to initiating a course of treatment, contraindications include: cardiovascular, renal or thyroid disease. All menstruating females should receive pregnancy tests, which if positive, is an absolute contraindication to treatment. All patients are to receive an initial EKG, electrolytes, BUN, creatinine, TSH, T3 and T4. Lithium blood levels are to be drawn, for example, at week four, eight and thereafter two times a year. Capsules could be customized to achieve maintenance/prevention lithium levels of 0.6-1.2 mEQ/L.

In particular embodiments, PCPs will be guided as to which drugs, at what dose, at exactly what frequency the medication should be administered. The methods described herein include a patient-friendly and physician-friendly method that utilizes single, color-coded bedtime capsules, that, for the first time, will make the treatment of mood disorders accessible to PCPs.

As previously described herein, mood spectrum disorder includes, but is not limited to Bipolar I Disorder, Bipolar II Disorder, Borderline Personality Disorder, Cyclothymia, Depressive Personality Disorder, Dysthymia, Eating Disorder, Major Depressive Disorder, Pre-Menstrual Dysphoric Disorder, Panic Disorder, Posttraumatic Stress Disorder, Schizoaffective Disorder, Seasonal Affective Disorder, Social Anxiety Disorder, Substance Use Disorder, and Treatment Resistant Depression. Many of these disorders are co-morbid and patients can often present with two or more of these conditions.

For example, patients who have “double depression,” (MDD and dysthymia together) constitute 40% of all MDD patients. It has been shown that at least 50% of all antidepressant medication fail to adequately treat MDD. This high prevalence of Treatment Resistant Depression suggests these patients are “pseudo-unipolar.” Manning el al., Clin. Psychiatry 4:142-150 (2002).

In particular embodiments, patients who fail to respond to antidepressant treatment should be treated with lithium and an antidepressant, such as an SSRI. In yet further embodiments, patients that present with any of the above listed disorders, except for Schizoaffective Disorder; Bipolar I Disorder, manic type; or Bipolar II, currently hypomanic; can all be initially treated with a single dose formulation of lithium and an SSRI. For those patients who are diagnosed with Schizoaffective Disorder; Bipolar I Disorder, manic type; Bipolar II, currently hypomanic, in other embodiments, these patients should first be treated with an atypical neuroleptic to stabilize their condition. The atypical neuroleptics are anxiolytic, mood stabilizing and can be chosen for bedtime sedation. Atypical neuroleptics are particularly indicated for manic, hypomanic, agitated, and psychotic patients as well as for those patients suffering from insomnia. Most patients treated with atypical neuroleptics will usually respond after a single dose. Once the patient is stabilized with the atypical neuroleptic, treatment can be followed by a combination of lithium and an SSRI in a single formulation co-administered with the atypical neuroleptic. In particular embodiments, a three-drug combination would be appropriate including lithium, an SSRI and an atypical neuroleptic. In particular embodiments, atypical neuroleptics would be appropriate for all phases of Bipolar I Disorder, BPD, GAD, PD, PTSD, all types of Schizoaffective Disorder, SUD and Social Anxiety Disorder. In yet further embodiments, the atypical neuroleptics can then be combined to with lithium and an SSRI to treat these disorders. In further embodiments where the combination of lithium, an SSRI, and an atypical neuroleptic fails to treat depressive symptomology, an antiseizure medication should be added. In particular embodiments, the addition of lamotrigine is appropriate for patients who continue to present with depressive symptomology. In other particular embodiments, the addition of topiramate is appropriate for those patients who continue to present with depressive symptomology and are also obese. In particular embodiments, treatment is then monitored and refined following symptom screen progress. Based on symptom screens and laboratory tests for serum blood levels of the pharmaceutical compounds described herein, either the pharmaceutical companies or pharmacists will be able to create appropriate dosages for patients in single formulations. For example, during the patient's follow-up visits, blood may be drawn to test lithium levels. In particular embodiments, the lithium prescription would be increased until therapeutic levels are reached. In another example, the patient would be asked to assess how they feel on a scale of 1 to 10 for various types of symptoms as described herein. Thus, a patient's progress is measured for each symptom at each follow-up visit with a “1” representing the patient's baseline symptomology to a “10” wherein the patient is no longer experiencing the symptom. According to the invention, “designer drugs” are created for every patient with their own uniquely titrated drug combination, contained in a single capsule that is designed to prevent relapse, recurrence or to treat acute conditions.

By customizing capsules and providing patients with the individualized dose combination they need, the patient and their PCP can collaborate in designing both acute and maintenance drug treatments. Quarterly lithium blood levels and annual EKGs for patients over forty years old will further limit possible long-term side effects.

Table A provides a list of the disorders described herein and some non-limiting, examples of compositions to be used to treat the disorders. In particular embodiments, a patient would be administered the mood screens. The PCP would base the diagnosis on the results of the mood screens. Once a diagnosis is made, particular compositions described herein are used to treat the patient. The daily dosage regimen begins with the initial dosages for the compounds described herein and are titrated based on follow-up visits with the patients. Once the patients reach remission they are placed on a single dosage formulation of their maintenance medications followed by regular follow-up consultations. In Table A, “Li” is meant to denote lithium. Lithium includes, but not limited to, long and short acting forms, oral and non-oral forms, lithium salts, lithium citrate, and lithium carbonate. All other compounds provided in the chart also comprise their long acting and short acting forms as well as oral and non-oral formulations.

TABLE A
Mood Spectrum Disorders and Recommended Non-Limiting
Treatment Compositions for Use in Treating Associated Mood Spectrum Disorders

| Mood Spectrum Disorders | Treatment Compositions |
|---|---|
| Bipolar I Disorder, depressed type | Li/SSRI; Li/SSRI/Antiseizure; Li/SSRI/Atypical |
| Acute and maintenance | Neuroleptic; Li/SSRI/Antipsychotic; |
| | Li/SSRI/Tricyclic Antidepressant; |
| | Li/SSRI/Bupropion; Li/SSRI/Tricyclic |
| | Antidepressant/Atypical Neuroleptic; |
| | Li/SSRI/Tricyclic Antidepressant/Atypical |
| | Neuroleptic/Antiseizure; Li/Bupropion; |
| | Li/Mirtazapine; Bupropion/Atypical |
| | Neuroleptic; Li/Atypical Neuroleptic; |
| | Li/Antiseizure; Li/Tricyclic Antidepressant; |
| | Bupropion/Tricyclic Antidepressant; |
| | Bupropion/Tetracyclic Antidepressant; |
| | Li/Antipsychotic; Li/MAOI; |
| | Li/Bupropion/Modafinil; |
| | Li/Bupropion/Lamotrigine; |
| | Bupropion/Lamotrigine; |
| Bipolar I Disorder, manic type | Li/SSRI; Li/SSRI/Atypical Neuroleptic; |
| Acute and maintenance | Li/SSRI/Antipsychotic; Li/Atypical Neuroleptic; |
| | Li/Antipsychotic; Li/Bupropion/Atypical |
| | Neuroleptic; Li/Bupropion/Antiseizure; |
| | Li/Bupropion/Antipsychotic; |
| | Li/Bupropion/Antiseizure/Antipsychotic; Li/ |
| | MAOI; Li/Bupropion/Lamotrigine; |
| | Bupropion/Lamotrigine; Li/Bupropion; |
| | Li/Mirtazapine; Bupropion/Antiseizure drug |
| Bipolar I Disorder, mixed type | Bupropion/Tricyclic Antidepressant; |
| Acute and maintenance | Bupropion/Tetracyclic Antidepressant; |
| | Li/Antipsychotic; Li/MAOI; |
| | Li/Bupropion/Modafinil; |
| | Li/Bupropion/Lamotrigine; |
| | Bupropion/Lamotrigine; Li/Bupropion; |
| | Li/Mirtazapine; Bupropion/Antiseizure drug |
| Bipolar Disorder II | Li/SSRI; Li/SSRI/Atypical Neuroleptic; |
| | Li/SSRI/Antipsychotic; Li/SSRI/Antiseizure; |
| | Li/Atypical Neuroleptic; Li/Antipsychotic; |
| | Li/Antiseizure; Li/MAOI; |
| | Li/Bupropion/Modafinil |
| Borderline Personality Disorder | Li/SSRI; Li/SSRI/Atypical Neuroleptic; |
| | Li/SSRI/Antipsychotic; Li/SSRI/Antiseizure; |
| | Li/Atypical Neuroleptic; Bupropion/Tricyclic |
| | Antidepressant; Bupropion/Tetracyclic |
| | Antidepressant; Li/Antipsychotic; Li/MAOI; |
| | Li/Bupropion/Modafinil; |
| | Li/Bupropion/Lamotrigine; |
| | Bupropion/Lamotrigine; Li/Bupropion; |
| | Li/Mirtazapine |
| Cyclothymia | Li/SSRI; Li/SSRI/Atypical Neuroleptic; |
| | Li/SSRI/Antipsychotic; Li/SSRI/Antiseizure; |
| | Li/Atypical Neuroleptic; Bupropion/Tricyclic |
| | Antidepressant; Bupropion/Tetracyclic |
| | Antidepressant; Li/Antipsychotic; Li/MAOI; |
| | Li/Bupropion/Modafinil; |
| | Li/Bupropion/Lamotrigine; |
| | Bupropion/Lamotrigine; Li/Bupropion; |
| | Li/Mirtazapine |
| Depressive Personality | Li/SSRI; Li/SSRI/Antiseizure; |
| Disorder | Li/SSRI/Bupropion; Bupropion/Tricyclic |
| | Antidepressant; Bupropion/Tetracyclic |
| | Antidepressant; Li/Antipsychotic; Li/MAOI; |
| | Li/Bupropion/Modafinil; |
| | Li/Bupropion/Lamotrigine; |
| | Bupropion/Lamotrigine; Li/Bupropion; |
| | Li/Mirtazapine |
| Dysthymia | Li/SSRI; Li/SSRI/Antiseizure; Li/SSRI/Atypical |
| | Neuroleptic; Li/SSRI/Antipsychotic; |
| | Li/Antiseizure; Li/Atypical Neuroleptic; |
| | Li/Antipsychotic; Li/Bupropion; |
| | Li/Antiseizure/Tricyclic Antidepressant; |
| | Li/Mirtazapine; Li/Tricyclic Antidepressant; |
| | Li/MAOI; Bupropion/Tricyclic Antidepressant; |
| | Bupropion/Tetracyclic Antidepressant; |
| | Li/Bupropion/Modafinil; |
| | Li/Bupropion/Lamotrigine; |
| | Bupropion/Lamotrigine |
| Eating Disorder | Li/SSRI; Li/SSRI/Atypical Neuroleptic; |
| | Li/SSRI/Atypical Neuroleptic/Antiseizure; |
| | Li/Atypical Neuroleptic; Li/Antipsychotic; |
| | Li/Tricyclic Antidepressant; Li/Antiseizure |
| General Anxiety Disorder | Li/SSRI; Li/SSRI/Atypical Neuroleptic; |
| Major Depressive Disorder | Li/SSRI/Antipsychotic |
| | Li/SSRI; Li/Tricyclic Antidepressant; Li/MAOI; |
| | Li/Antiseizure; Li/Tricyclic |
| | Antidepressant/Antiseizure; Li/Bupropion; |
| | Li/Mirtazapine; Li/SSRI/Tricyclic |
| | Antidepressant; Li/SSRI/Tricyclic |
| | Antidepressant/Atypical Neuroleptic; |
| | Li/SSRI/Tricyclic Antidepressant/Antiseizure; |
| | Li/SSRI/Tricyclic Antidepressant/Atypical |
| | Neuroleptic/Antiseizure; |
| | Li/Bupropion/Modafinil |
| Panic Disorder | Li/SSRI; Lithium/SSRI/Atypical Neuroleptic |
| Posttraumatic Stress Disorder | Li/SSRI; Li/SSRI/Atypical Neuroleptic; |
| | Li/Antiseizure |
| Pre-Menstrual Dysphoric Disorder | Li/SSRI; Li/SSRI/Antiseizure; Li/SSRI/Atypical |
| | Neuroleptic; Li/Antiseizure; Li/Atypical |
| | Neuroleptic; Li/Bupropion |
| Schizoaffective Disorder, depressed | Li/Atypical Neuroleptic; Li/Antipsychotic; |
| | Li/SSRI/Atypical Neuroleptic; |
| | Li/SSRI/Antipsychotic; |
| | Li/SSRI/Antipsychotic/Antiseizure; |
| | Li/SSRI/Atypical Neuroleptic/Antiseizure; |
| | Li/SSRI/Bupropion; |
| | Li/SSRI/Bupropion/Atypical Neuroleptic; |
| | Li/SSRI/Bupropion/Antipsychotic |
| Schizoaffective Disorder, | Li/Atypical Neuroleptic; Li/Antipsychotic; |
| manic | Li/Atypical Neuroleptic/Antiseizure; |
| Acute and maintenance | Li/Antipsychotic/Antiseizure; |
| | Li/Bupropion/Atypical Neuroleptic; |
| | Li/Bupropion/Antipsychotic; |
| | Li/Bupropion/Atypical Neuroleptic/Antiseizure; |
| | Li/Bupropion/Antipsychotic/Antiseizure; |
| | Li/SSRI/Atypical Neuroleptic; |
| | Li/SSRI/Antipsychotic; Li/SSRI/Antiseizure; |
| | Li/SSRI/Atypical Neuroleptic/Antipsychotic; |
| | Bupropion/Antiseizure drug |
| Schizoaffective Disorder, | Bupropion/Antiseizure drug; Li/Antipsychotic; |
| mixed | Li/Atypical Neuroleptic/Antiseizure; |
| Acute and maintenance | Li/Antipsychotic/Antiseizure; |
| | Li/Bupropion/Atypical Neuroleptic; |
| | Li/Bupropion/Antipsychotic; |
| | Li/Bupropion/Atypical Neuroleptic/Antiseizure; |
| | Li/Bupropion/Antipsychotic/Antiseizure; |
| | Li/SSRI/Atypical Neuroleptic; |
| | Li/SSRI/Antipsychotic; Li/SSRI/Atypical |
| | Neuroleptic/Antiseizure; |
| | Li/SSRI/Antipsychotic/Antiseizure |
| Seasonal Affective Disorder | Li/SSRI; Li/SSRI/Atypical Neuroleptic; |
| | Li/SSRI/Antiseizure; Li/Bupropion; |
| | Li/Bupropion/Atypical Neuroleptic; Li/Tricyclic |
| | Antidepressant |
| Social Anxiety Disorder | Li/SSRI; Li/SSRI/Atypical Neuroleptic; |
| | Li/SSRI/Antipsychotic |
| Substance Use Disorder | Li/Bupropion; Li/Bupropion/Atypical |
| | Neuroleptic; Li/Bupropion/Antipsychotic; |
| | Li/Bupropion/Antiseizure; Li/SSRI; |
| | Li/SSRI/Antipsychotic; Li/SSRI/Antiseizure; |
| | Li/SSRI/Atypical Neuroleptic; |
| | Li/SSRI/Antiseizure/Atypical Neuroleptic; |
| | Li/SSRI/Tricyclic Antidepressant; Li/MAOI; |
| | Li/Atypical Neuroleptic; Bupropion/Tricyclic |
| | Antidepressant; Bupropion/Tetracyclic |
| | Antidepressant; Li/Antipsychotic; |
| | Li/Bupropion/Modafinil; |
| | Li/Bupropion/Lamotrigine; |
| | Bupropion/Lamotrigine; Li/Mirtazapine |
| Treatment Resistant | Li/SSRI; Li/SSRI/Atypical Neuroleptic; |
| Depression Acute and maintenance | Li/SSRI/Antiseizure; Li/SSRI/Tricyclic |
| | Antidepressant; Li/Bupropion; |
| | Li/Bupropion/Atypical Neuroleptic; |
| | Li/Bupropion/Antiseizure; Li/MAOI; |
| | Li/Tricyclic Antidepressant; |
| | Li/Tricyclic Antidepressant/Atypical |
| | Neuroleptic; Li/Tricyclic |
| | Antidepressant/Atypical |
| | Neuroleptic/Antiseizure; Li/SSRI/Venlafaxine; |
| | Li/SSRI/Bupropion; |
| | Li/SSRI/Venlafaxine/Antiseizure; |
| | Li/SSRI/Bupropion/Antiseizure; |
| | Bupropion/Tricyclic Antidepressant; |
| | Bupropion/Tetracyclic Antidepressant; |
| | Li/Antipsychotic; Li/Bupropion/Modafinil; |
| | Li/Bupropion/Lamotrigine; Bupropion/ |
| | Lamotrigine; Li/Mirtazapine |

### EXAMPLES

### Example 1

Mood Screen for Bipolar Disorder. Hirshfeld et al., Development and validation of a screening instrument for bipolar spectrum disorder: the Mood Disorder Questionnaire, Am. J. Psychiatry, 157:1873-1875 (2000). In order to screen positively for Bipolar Disorder, a patient must: (1) answer “yes” to at least 7 of the 13 parts in question 1; (2) answer “yes” to question 2; and (3) have at least a “moderate” or “serious” degree of functional impairment on question 3.

Question 1: Has there ever been a period of time when you were not your usual self and
a) you felt so good or so hyper that other people thought you were not your normal self or you were so hyper that you got into trouble?
b) you were so irritable that you shouted at people or started fights or arguments?
c) you felt much more self-confident than usual?
d) you got much less sleep than usual and found you didn't really miss it?
e) you were much more talkative or spoke much faster than usual?
f) thoughts raced through your head or you couldn't slow your mind down?
g) you were so easily distracted by things around you that you had trouble concentrating or staying on track?
h) you had much more energy than usual?
i) you were much more active or did many more things than usual?
j) you were much more social or outgoing than usual, for example you telephoned friends in the middle of the night?
k) you were much more interested in sex than usual?
l) you did things that were unusual for you or that other people might have thought were excessive, foolish, or risky?
m) spending money got you or your family into trouble?

Question 2: If you answered YES to more than one of the above, have several of these ever happened during the same period of time?

Question 3: How much of a problem did any of these cause you—like being unable to work; having family, money or legal troubles, getting into arguments or fights?Select one . . . no problem . . . minor problem . . . moderate problem . . . serious problem.

### Example 2

### Mood Screen For Dysthymia

Scoring 3 or more positive answers, meet with patient to confirm diagnosis. Scoring 4 or more positive answers, initiate treatment with mood stabilizer and SSRI combination, such as lithium and citalopram (SERATROL).
1. Do you often get the “blues”?
2. Are you a perfectionst?
3. Do you tent to “make mountains out of mole hills”?
4. Are you overly sensitive to criticism by others, even if they are wrong?
5. Do you often have low self esteem?
6. Do you tend to be a worrier and a pessimist rather than an optimist?

### Example 3

### Mood Screen For Affective Disorder

Score each question with either a 0=never, 1=sometimes, or 2=frequently.
1. Are you moody? (Do you frequently shift from feeling really good to down in the dumps?)
2. Do you have a “short fuse?”(Are you irritable or angry?)
3. Do you talk fast? (Can bursts of energy keep you up all night?)
4. Are you impulsive? (Do you gamble or say or do things you later regret?)
5. Do you ever feel like life isn't worth living?
6. Have you thought about suicide or made a past attempt?
7. Sometimes I don't even feel like getting out of bed. I feel sad, hopeless and helpless.
8. Does drinking alcohol “help keep the lid on” these feelings?
9. Might depression or mood disorder run in your family?

Add up) score. If score is equal to or greater than 7, diagnose and treat mood disorder by assessing the answers as follows:

A. Did the patient answer questions 1-4 and 8-9 with a score of 5 or greater? If yes, then probable diagnosis of bipolar I disorder, manic depression.

B. Did the patient answer questions 5-9 with a score of 4 or greater? If yes, then probable diagnosis of MDD.

C. Did the patient answer at least 2 questions positively (score of 1 or 2) from questions 1-3?

If yes, did patient answer at least 2 questions positively from questions 5-8?

If yes, then probable diagnosis of bipolar, depressed type.

### Example 4

SERATROL 20/300
Color: Red
Directions: SERATROL Red Tablet containing 20 mg Citalopram with 300 mg Lithium XR; 1 tablet at bedtime for 2 weeks; re-assess symptom questionnaire after 2 weeks
Seratrol 30/600
   Color: Yellow
   Directions: SERATROL Yellow Tablet containing 30 mg Citalopram with 600 mg Lithium XR; 1 tablet at bedtime for 4 weeks; re-assess symptom questionnaire after 4 weeks

SERATROL 40/900
Color: Green
Directions: SERATROL Green Tablet containing 40 mg Citalopram with 900 mg Lithium XR; 1 tablet at bedtime everyday as a Maintenance Dose.

### Example 5

Patient A presented with dysthymia, her maintenance dose green capsule of lithium with citalopram can include 40 mg of citalopram 750 mg of lithium extended release.

### Example 6

A bipolar I manic patient might begin treatment with a capsule containing only 600 mg of lithium in a single dosage formulation and may end treatment with a dose that includes 30 mg of citalopram and 1200 mg of lithium combined in a single dosage formulation, depending on the patient's blood lithium levels and symptom responsivity.

### Example 7

A patient diagnosed with dysthymia can begin treatment with 300 mg of lithium extended release and 20 mg of citalopram in a single dosage formulation and end treatment with 600 mg of lithium and 30 mg of citalopram in a single dosage formulation.

### Example 8

A patient diagnosed with borderline personality disorder can begin treatment with 300 mg of lithium extended release and 20 mg of citalopram in a single dosage formulation and end treatment with 900 mg of lithium extended release and 20 mg of citalopram as their maintenance dose in a single dosage formulation.

### Example 9

A 50 year old male physician presented with signs and symptoms of MDD. He had experienced a similar episode one year prior and one past MDD episode in medical school. The patient reported he was hypomanic over the summer months preceding cycling into his current depression. Hypomania was manifested with increased irritability, poor insight and judgment, hyperactivity, aggression and an altered speech patter. The Affective Disorder Screen (See Example 3) indicated a diagnosis of Bipolar II disorder. During the first week the patient was treated with a combination of 300 mg lithium extended release and 150 mg of bupropion per day. The combination can be provided to the patient in single dosage formulation. During the second week, his daily dosages were doubled to 600 mg and 300 mg of lithium extended release and bupropion, respectively. This dosage can also be provided to the patient in a single dosage formulation. On the third week, the patient's daily dosages were increased to 900 mg and 300 mg of lithium extended release and bupropion, respectively. The regimen of the third week can also be provided in a single dosage formulation. On the fourth week, the patient's depression persisted at a rating of 4-5 on a scale of 1 to 10. The lithium plasma level was 0.8 mEq/L. Therefore, the patient's dosage was changed to 900 mg lithium and 450 mg bupropion. By the end of week 5, the patient was euthymic and fully functional. He has remained on his maintenance dose for over five years without any clinically significant mood cycling.

### Example 10

A 49 year old single female professor presented with the chief complain, “My life is meaningless.” Symptoms included decreased energy and motivation, anhedonia, obsessive thinking, hopelessness, impaired self-esteem, guilt and irritable bowel syndrome. The patient experienced general anxiety disorder with an average of three panic attacks per month. Concentration and irritability were unimpaired. Sleep and appetite were normal. There was no reversal of diurnal variation. The patient failed to meet DSM-IV criteria for MDD, but scored 5 of 6 on the Dysthymia Screen (See Example 2). The patient indicated that she “felt depressed ever since I can remember.” There was no discrete history of MDD or mania. On year prior, the patient had been treated with 40 mg of fluoxetine (PROZAC), which decreased depressive symptoms, but “dulled” the patient cognitively, and was subsequently discontinued. The patient was started on a daily dosage of 300 mg of lithium and 10 mg of escitalopram. This daily dosage regiment can be administered to the patient as a single dosage formulation. By the end of week 1, the patient had no irritable bowel syndrome for the first time in seven years. On week two the dosage was increased to 600 mg of lithium and 10 escitalopram, with blood tested to ensure lithium blood level was at 0.6-1.2 mEq/L. The dosage regimen for week two can also be administered as a single dosage formulation. By the end of week 2, anxiety was at baseline. On week three, the patient's daily dosage was increased to 900 mg lithium and 20 mg of escitalopram. This dosage regimen can be administered to the patient as a single dosage formulation. By the end of week 3, depressive symptoms were in full remission. The patient stated, “I've never felt this well before.” Symptom relief enabled a successful two-year psychodynamic psychotherapy.

### Example 11

A 39 year old professor had been on Wellbutrin SR (bupropion hydrochloride) for MDD, first episode, under his prior psychiatrist with modest symptomatic success. The patient presented with decreased energy and motivation, insomnia, feelings of hopelessness, worthlessness, guilt and anhedonia. The patient also manifested Obsessive Compulsive Disorder, Generalized Anxiety Disorder and Panic Disorder. There was no suicidal ideation. Bupropion was increased to 450 mg without therapeutic gain. The diagnosis of treatment resistant depression was made. The patient was cross-titrated, initiating lithium and escitalopram simultaneously, while down-titrating bupropion from 450 mg to 0 mg over four weeks. During week 1, the daily dosage regimen was 300 mg lithium extended release, 10 mg escitalopram and 450 mg bupropion. This daily dosage regimen can be administered to the patient in a single dosage formulation. During week two, the daily dosage regimen was 600 mg lithium extended release, 10 mg escitalopram, and 300 mg bupropion. This daily dosage regimen for week two can also be administered to the patient as a single dosage formulation. During week three, the daily dosage regimen was 900 mg lithium extended release, 0 mg escitalopram, and 150 mg bupropion. The daily dosage regiment for the third week can be formulated into single dosage formulation. During week 4, the daily dosage regiment was 900 mg lithium extended release, 20 mg escitalopram, and 0 mg bupropion. Escitalopram had been increased because of continuing obsessive compulsive disorder. This regimen may also be formulated into single dosage formulation for treatment of the patient. By week 4, the patient had a lithium blood level of 0.74 mEq/L, the patient's mood was stable and euthymic with signs and symptoms of MDD in full remission. His OCD had improved by 70%.

### Example 12

Method for treating Dysthymia. Mr. A is a 52 year old male who goes to See his PCP. The PCP provides Mr. A with a Dysthymia Screen, a Bipolar Disorder Screen and an Affective Disorder Screen. He scores 5 of 6 on the Dysthymia Screen, 4 of 13 on the Bipolar Disorder Screen and 5 on the Affective Disorder Screen. On the basis of these scores the PCP can conclude Mr. A has Dysthymia and does not have Bipolar illness. Mr. A confirms the doctor's diagnosis of Dysthymia by saying, “I've been this way my whole life.” Mr. A had been treated unsuccessfully 10 years prior with both 60 mg of fluvoxamine and on another occasion with 40 mg of paroxetine. Mr. A was resigned to his illness saying, “I guess this is just the way life is.” Mr. A's history of failure to respond to two prior trials of antidepressant medication suggested lithium augmentation.

The PCP can choose to use SERATROL because of its efficacy for treating Dysthymia. Mr. A has no cardiovascular, renal or thyroid disease and all of his laboratory tests, including EKG, electrolytes, BUN, creatinine, TSH, T3 and T4 were within normal limits. The PCP can initiate treatment with a Red capsule of SERATROL containing 300 mg lithium extended release with 20 mg of citalopram. Since the PCP is aware of the patient's prior history of failure to improve on significant doses of two SSRIs the patient would then be instructed to take one capsule at bedtime for one week and if there were no side effects, to then to take two capsules at bedtime for the second week. Therefore, during week two, the patient's daily dosage is 2 Red capsules containing a total of 600 mg lithium extended release and 40 mg of citalopram.

At the end of week 2, the patient returns for follow-up. Mr. A reports, “I don't know why, but I'm just starting to feel more motivated.” Mr. A rates his dysthymia compared to his initial baseline visit as improved by three on a scale of ten. The PCP takes a blood sample to test for Mr. A's lithium levels, which is 0.54 mEQ/L. On average, 600 mg/day of lithium produces a sub-therapeutic level in the average adult male. Maintenance lithium level is between 0.6-0.8 mEq/1. is ideal, while acute levels can go as high as 1.2 mEq/L.

The PCP would then prescribe the green capsule of SERATROL, which contains 900 mg lithium extended release and 40 mg of citalopram. Mr. A is to take one capsule at bedtime.

On Mr. A's next follow-up visit at the end of week 4, Mr. A reported that he had experienced, on a few occasions a sense of pleasure that he had never felt before. He now rates his improvement as 6 on a scale of 10.

On the patient's follow-up visit during week five, Mr. A looks like a different man. Rather than having a closed physical demeanor, he stood straighter and taller and smiled for the first time. He rated his improvement as 8-9 out of 10. Mrs. A accompanied her husband at his second follow-up visit and was thrilled with the personality change in her husband. The PCP concludes that Mr. A's mood disorder is in full remission.

The PCP would then prescribe the SERATROL Green capsule treatment dosage formulation with 900 mg lithium extended release and 40 mg citalopram as the lifetime maintenance dose. Mr. A then has biannual lithium blood levels and an annual EKG. After three years of follow-up, Mr. A has remained symptom free.

### Example 13

Method for treating Bipolar Disorder. Ms. B is a 73 year old female who goes to See her PCP. The PCP administers the Bipolar Disorder Screen, the Dysthymia Screen and the Affective Disorder Screen. Ms. B answers yes to 8 of 13 questions on the Bipolar Disorder Screen. Her history reveals that she has had at least three prior depressive episodes that met with DSM-IV criteria for MDD. Ms. B also reports that she is unable to sleep at night, has racing thoughts, has generalized anxiety, is unable to concentrate, and is feeling Dysphoric. She appears somewhat agitated and irritable. There is no prior history, however, of mania or hypomania. Laboratory tests are run on Ms. B. There were no cardiovascular, renal or thyroid disease and all other laboratory tests, including EKG, electrolytes, BUN, creatinine, TSH, T3 and T4 were within normal limits for a woman of her age. Ms. B should be treated with a drug that includes lithium and an atypical neuroleptic.

During week one, the PCP decides to prescribe a single red capsule containing 300 mg lithium and 50 mg of quetiapine to be taken at bedtime. Quetiapine is associated with less daytime sedation when prescribed at bedtime and is efficacious for treating insomnia. Additionally, quetiapine is associated with less weight gain, extrapyramidal symptoms, dystonia and tardive dyskinesia than with the other sedative atypical neuroleptic, olanzapine.

During week 2, on a follow-up visit, Ms. B reports that her agitation, racing thoughts and insomnia have all improved to a 3 on a scale of 1 to 10. She also reports, however, that her irritability, poor concentration and dysphoric feelings persist at level 7 on a scale of 1 to 10. The PCP prescribes an increase in her lithium and quetiapine and is therefore prescribed a yellow capsule with 450 mg of lithium extended release and 100 mg of quetiapine.

During week 3, Ms. B reports that her agitation, racing thoughts and insomnia have improved to a 6 on a scale of 1 to 10, but her dysphoria persists. The PCP takes a blood sample to assess her lithium blood levels and her lithium levels are 0.49 mEQ/L. The PCP increases her lithium dosage, maintains her quetiapine dosage and adds citalopram. The PCP prescribes Ms. B a green capsule containing 600 mg lithium extended release with 100 mg quetiapine and 20 mg citalopram.

During week 4, Ms. B reports that her hypomanic symptoms have improved to an 8 on a 1 to 10 scale, but her dysphoria persists. The PCP takes a blood sample to assess her lithium levels and finds it to be 0.7 mEq/L. The PCP decides to maintain Ms. B on her green capsule since the effects of the SSRI could take at least 2 weeks to be clinically effective.

During week 5, Ms. B says her dysphoria has improved to an 8 on a scale of 1 to 10, she is no longer agitated or anxious, has not had insomnia, and her racing thoughts have ceased. The PCP decides to keep her green capsule prescription the same.

During week 7, Ms. B has a follow-up EKG and claims that she is feeling like a 9 on a scale of 1 to 10 on her dysphoria with no hypomanic symptoms. The PCP decides to maintain Ms. B on her current prescription and follows up with Ms. B 1 month later.

During week 11, Ms. B continues to do well with no hypomanic symptoms and no dysphoria. She is to return in two months for a follow up, then again 3 months after that and once every quarter for a year, then biannually after that. Her lithium levels are checked biannually and an annual EKG is performed.

### Example 14

Method for treating Treatment Resistant Depression. Ms. S, a 43-year old elementary school teacher, is experiencing her third episode of Treatment Resistant Depression over the past four years. During her initial episode of Treatment Resistant Depression, Ms. S was hospitalized for depression with accompanying suicidality. The patient was discharged from the hospital on 150 mg ZOLOFT, which was titrated up to 225 mg. Ms. S showed a partial response. Following her second episode of Treatment Resistant Depression, an SNRI, CYMBALTA, was used to augment Ms. S's Zoloft. Despite titrating CYMBALTA to its maximum amount of 120 mg, Ms. S's depression resolved by approximately 50%.

Week 1: Given that the chance of recurrence when a patient achieves response rather than remission is extremely high, Ms. S was begun on WELLBUTRIN XL/desipramine in single dosage formulation. Based on her HAM-D GAF scores, Ms. S is diagnosed with her third episode of Treatment Resistant Depression. Ms. S denies suicide plans and indicates little to no improvement with her prior CYMBALTA augmentation. Outpatient treatment is initiated by continuing her ZOLOFT at 250 mg while beginning 150 mg WELLBUTRIN XL/50 mg desipramine in single dosage formulation.

Week 2: The patient no longer manifests suicidal ideation and reports decreased depression and anhedonia with an increase in energy.

Week 3: WELLBUTRIN XL remains at 150 mg while desipramine is increased to 100 mg.

Week 4: The patient reports full remission of symptoms as indicated by a normal HAM-D and GAF scores. Ms. S is continued on this dosage as a lifetime regimen given her past suicidality and the number of episodes of mood disorder.

Week 6: The patient is reassessed. Depression scales were all within normal limits.

Week 10, 14 and 16 all continue to indicate full remission.

Ms. S has since been followed quarterly for twenty-eight months without recurrence.

### Example 15

Method for treating Bipolar T. manic type. Mr. Jones, 59-year old, presents with Bipolar I, manic type. According to his PCP. the patient has likely experienced one period of depression twenty years ago and two recent manic episodes for which Mr. Jones has failed to follow through with the psychiatric consult arranged by his PCP.

Week 1: Mr. Jones is brought to treatment by his wife who confirms that once again her husband is irritable, angry, hardly sleeping and manifesting increased libido. Mrs. Jones indicates that her husband, “punched the boss,” and was fired last week. Mood screens confirm the diagnosis of Bipolar I, manic type. Mental status examination demonstrates auditory hallucinations and homicidal ideation along with the symptoms listed above. Mr. Jones is begun on single dosage formulation containing 150 mg lithium ER/2 mg perphenazine twice a day.

Week 2: The patient is no longer manifesting the signs and symptoms of mania, however some depression is noted along with moodiness and moderate irritability. Mr. Jones's perphenazine dose remains on 4 mg while lithium is increased to 900 mg. Therefore, his single dosage formulation capsules are 450 mg lithium ER/2 mg perphenazine twice a day.

Week 3: The patient's lithium blood level has returned as 0.8 mEq/L. Mr. Jones reports a fine motor tremor, making writing difficult. 2 mg COGENTIN [Not previously disclosed] is prescribed at bedtime. There are no signs or symptoms of mania or moodiness.

Week 4: Mr. And Mrs. Jones indicate that the patient is symptom free and without tremor. Therefore, Mr. Jones' single dose maintenance formulation is 450 mg lithium ER/2 mg perphenazine bid. He continues on 2 mg COGENTIN at bedtime.

The patient is followed at week 6, 10, and 14 without evidence of mania nor depression.

### Example 16

Method for treating Dysthymia and Bipolar II. Mrs. E, a 34-year old housewife and mother of two, presents with her first episode of depression. On mood disorder screening, the patient tests positive for Dysthymia and Bipolar II. On clinical interview, Mrs. E reports having the blues since her teenage years with more serious depressions lasting anywhere from two weeks to two months, perhaps four or five times in her life period.

Week 1: Mrs. E is begun on single dosage formulation of 300 mg lithium ER 150 mg/WELLBUTRIN XL.

Week 2: WELLBUTRIN is increased to 300 mg and lithium to 600 mg.

Week 3: The patient reports 70% improvement in her depressive symptoms; however, she continues with low energy and low motivation. Mrs. E's WELLBUTRIN XL is increased to 450 mg while her lithium ER is increased to 900 mg.

Week 4: The patient indicates she is no longer depressed but continues with low energy. Lithium level is 0.9 mEq/L. The determination is made to augment her lithium/WELLBUTRIN capsule with PROVIGIL 200 mg.

Week 5: Mrs. E states, “1 have a lot more energy.” She continues with stable, euthymic mood. Clinically, it is unclear whether Mrs. E might benefit from additional PROVIGIL. Therefore, the patient is treated with 450 mg lithium ER/PROVIGIL 400 mg once in the morning and 450 mg lithium ER/450 mg WELLBUTRIN XL once at night.

Week 6: Mrs. E states, “I've never felt more energetic and less depressed in my whole life.” Her maintenance dose is the equivalent of her final treatment titration dose.

The patient is followed monthly for five months. Her mood is stable and euthymic without any evidence of any hypomania.

### Example 17

Method for treating Dysthymia and Bipolar 11. Mr. J is a 39-year old construction worker. He presents with severe alcohol abuse and Borderline Personality Disorder as manifested by mood instability, low self-esteem, angry outbursts and superficially cutting on his wrists. Mr. J's alcoholism has increased from one to three 6-packs per week since his divorce one month ago. This patient has had two prior episodes of depression both associated with separation from his wife. On those occasions, he had only a partial response to the combination of an SNRI (CYMBALTA) in combination with WELLBUTRIN.

On mental status examination, the patient presents with tearful outbursts, pessimism, depression, and extreme feelings of separation anxiety. The patient is diagnosed with Substance Use Disorder plus Dysthymia on Axis I, and Borderline Personality Disorder on Axis II. Mood screens ruled out major depression and Bipolar Disorder and indicated Dysthymia.

Week 1: The patient agrees to abstinence during a one-month trial period of medication. Mr. J states, “1 know that alcohol only makes me more depressed and angrier so I'm willing to give your treatment a try.” The patient is begun on a single dosage formulation of 150 mg WELLBUTRIN XL and 50 mg amoxapine, a tetracyclic antidepressant. Mr. J is also instructed to immediately call his doctor if he has any suicidal thoughts.

Week 2: Patient reported mild decrease in depression and confirmed complete abstinence. He denied any suicidal ideation. Mr. J's dosage was changed to 300 mg WELLBUTRIN/100 mg amoxapine. Additionally, the patient continued self-mutilation.

Week 3: The patient reported no incidence of self-mutilation. The patient reported mild dry mouth and constipation earlier in the week, which was resolving. He also noted an increase in energy and a decrease in his desire to use alcohol. Depression was rated at 8.5/10 on week 1 and was reduced to 6/10 at this point in his treatment. Medication was left unchanged to allow for adequate duration for response.

Week 4: Mr. J reported no changes from the following week. Therefore, WELLBUTRIN XL was increased to 450 mg and amoxapine was continued at 100 mg.

Week 5: Mr. J noted depression to be at 3.5/10 with continued decreased cravings for alcohol. WELLBUTRIN XL is continued at 450 mg and amoxapine was increased to 200 mg.

Week 6: Mr. J stated “I've never felt this good before in my life.” He noted no depression for the last four days of the week. Mr. J further stated, “I feel like a whole person; 1 don't feel anxious when I'm by myself.” Mr. J was assessed to have achieved full remission and was continued on his most recent medication regimen and followed monthly, maintaining remission over the following seven months.

I claim:

1. A composition comprising bupropion and a tricyclic antidepressant formulated into a single dosage formulation.

2. The composition of claim 1, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

3. A composition comprising bupropion and a tetracyclic antidepressant formulated into a single dosage formulation.

4. The composition of claim 3, wherein the tetracyclic antidepressant is selected from the group consisting of amoxapine and maprotiline.

5. A composition comprising lithium and an antipsychotic drug formulated into a single dosage formulation.

6. The composition of claim 5, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

7. A composition comprising lithium and a monoamine oxidase inhibitor formulated into a single dosage formulation.

8. The composition of claim 7, wherein the monoamine oxidase inhibitor is selegiline.

9. A composition comprising lithium, bupropion, and modafinil in a single dosage formulation.

10. A composition comprising lithium, bupropion, and lamotrigine in a single dosage formulation.

11. A composition comprising bupropion and lamotrigine in a single dosage formulation.

12. A composition comprising bupropion and an antiseizure drug in a single dosage formulation.

13. The composition of claim 12, wherein the antiseizure drug is selected from the group consisting of oxcarbazepine, lamotrigene, topiramate, divalproex sodium, levetiracetam, gabapentin, tiagabine, and zonisamide.

14. A method for treating a patient for a mood spectrum disorder comprising administering to the patient an effective amount of bupropion and a tricyclic antidepressant formulated into a single dosage formulation.

15. The method of claim 14, wherein the tricyclic antidepressant is selected from the group consisting of desipramine, nortriptyline, amitriptyline hydrochloride, ,clomipramine hydrochloride, doxepin hydrochloride, imipramine pamoate, trimipramine maleate, and protriptyline.

16. The method of claim 14, further comprising diagnosing the patient by using a mood disorder screen.

17. A method for treating a patient for a mood spectrum disorder comprising administering to the patient an effective amount of bupropion and a tetracyclic antidepressant formulated into a single dosage formulation.

18. The method of claim 17, wherein the tetracyclic antidepressant is selected from the group consisting of amoxapine and maprotiline.

19. The method of claim 17, further comprising diagnosing the patient by using a mood disorder screen.

20. A method for treating a patient for a mood spectrum disorder comprising administering to the patient an effective amount of lithium and an antipsychotic drug formulated into a single dosage formulation.

21. The method of claim 20, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

22. The method of claim 20, further comprising diagnosing the patient by using a mood disorder screen.

23. A method for treating a patient for a mood spectrum disorder comprising administering to the patient an effective amount of lithium and an monoamine oxidase inhibitor formulated into a single dosage formulation.

24. The method of claim 23, wherein the monoamine oxidase inhibitor is selegiline.

25. The method of claim 23, further comprising diagnosing the patient by using a mood disorder screen.

26. A method for treating a patient for a mood spectrum disorder comprising administering to the patient an effective amount of lithium, bupropion, and modafinil formulated into a single dosage formulation.

27. The method of claim 26, further comprising diagnosing the patient by using, a mood disorder screen.

28. A method for treating a patient for a mood spectrum disorder comprising administering to the patient an effective amount of lithium, bupropion, and lamotrigine formulated into a single dosage formulation.

29. The method of claim 28, further comprising diagnosing the patient by using a mood disorder screen.

30. A method for treating a patient for a mood spectrum disorder comprising administering to the patient an effective amount of bupropion and lamotrigine formulated into a single dosage formulation.

31. The method of claim 30, further comprising diagnosing the patient by using a mood disorder screen.

32. A method for treating a patient for a mood spectrum disorder comprising administering to the patient an effective amount of bupropion and an antiseizure drug formulated into a single dosage formulation.

33. The method of claim 32 wherein the antiseizure drug is divalproex sodium.

34. The method of claim 32, further comprising diagnosing the patient by using a mood disorder screen.

35. A method for treating a patient for Bipolar I disorder, depressed type by administering an effective amount of a composition comprising one of the following combinations:
(a) bupropion and a tricyclic antidepressant;
(b) bupropion and a tetracyclic antidepressant;
(c) lithium and an antipsychotic drug;
(d) lithium and a monoamine oxidase inhibitor;
(e) lithium, bupropion, and modafinil;
(f) lithium, bupropion, and lamotrigine;
(g) bupropion and lamotrigene;
(h) lithium, a selective serotonin reuptake inhibitor and an antiseizure drug;
(i) lithium, a selective serotonin reuptake inhibitor and an atypical neuroleptic;
(j) lithium, a selective serotonin reuptake inhibitor and an antipsychotic drug;
(k) lithium, a selective serotonin reuptake inhibitor and a tricyclic antidepressant;
(l) lithium, a selective serotonin reuptake inhibitor and bupropion;
(m) lithium, a selective serotonin reuptake inhibitor, a tricyclic antidepressant, and an atypical neuroleptic;
(n) lithium, a selective serotonin reuptake inhibitor, a tricyclic antidepressant, an atypical neuroleptic, and an antiseizure drug;
(o) lithium and bupropion;
(p) lithium and mirtazapine;
(q) bupropion hydrochloride and an atypical neuroleptic; and
(r) lithium and a tricyclic antidepressant.

36. The method of claim 35, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

37. The method of claim 35, wherein the tetracyclic antidepressant is selected from the group consisting of amoxapine and maprotiline.

38. The method of claim 35, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

39. The method of claim 35, wherein the monoamine oxidase inhibitor is selected from the group consisting of phenelzine sulfate, tranylcypromine sulfate, selegiline.

40. The method of claim 35, comprising a selective serotonin reuptake inhibitor selected from the group consisting of citalopram, escitalopram, paroxetine, sertraline, fluoxetine, and duloxetine.

41. The method of claim 35, comprising an antiseizure drug selected from the group consisting of oxcarbazepine, lamotrigene, topiramate, divalproex sodium, levetiracetam, gabapentin. tiagabine, and zonisamide.

42. The method of claim 35, comprising an atypical neuroleptic selected from the group consisting of aripiprazole, quetiapine fumarate, risperidone, ziprasidone, and olanzapine.

43. A method for treating a patient for Bipolar I disorder, manic type, by administering an effective amount of a composition comprising one of the following combinations:
(a) lithium, a selected serotonin reuptake inhibitor, and an atypical neuroleptic;
(b) lithium, a selected serotonin reuptake inhibitor, and an antipsychotic drug;
(c) lithium and an antipsychotic drug;
(d) lithium, bupropion, and an atypical neuroleptic;
(e) lithium, bupropion and an antiseizure drug;
(f) lithium, bupropion, and an antipsychotic drug;
(g) lithium, bupropion, an antiseizure drug, and an antipsychotic drug;
(h) lithium and a monoamine oxidase inhibitor;
(i) lithium, bupropion, and lamotrigine;
(j) bupropion and lamotrigine;
(k) lithium and bupropion;
(l) lithium and mirtazapine; and
(m) bupropion and an antiseizure drug.

44. The method of claim 43, wherein the selective serotonin reuptake inhibitor is selected from the group consisting of citalopram, escitalopram, paroxetine, sertraline, fluoxetine, and duloxetine.

45. The method of claim 43, wherein the atypical neuroleptic is selected from the group consisting of aripiprazole, quetiapine fumarate, risperidone, ziprasidone, and olanzapine.

46. The method of claim 43, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

47. The method of claim 43, wherein the monoamine oxidase inhibitor is selected from the group consisting of phenelzine sulfate, tranylcypromine, and selegiline.

48. The method of claim 43, wherein the antiseizure drug is selected from the group consisting of oxcarbazepine, lamotrigene, topiramate, divalproex sodium, levetiracetam, gabapentin, tiagabine, and zonisamide.

49. A method for treating a patient for Bipolar I disorder, mixed type, by administering an effective amount of a composition comprising one of the following combinations:
(a) bupropion and a tricyclic antidepressant;
(b) bupropion and a tetracyclic antidepressant;
(c) lithium and an antipsychotic drug;
(d) lithium and monoamine oxidase inhibitor;
(e) lithium, bupropion, and modafinil;
(d) lithium, bupropion, and lamotrigine;
(g) bupropion and lamotrigine;
(h) lithium and bupropion;
(i) lithium and mirtazapine; and
(j) bupropion and an antiseizure drug.

50. The method of claim 49, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

51. The method of claim 49, wherein the tetracyclic antidepressant is selected from the group consisting of amoxapine and maprotiline.

52. The method of claim 49, wherein the antipsychotic drug is selected from the group consisting of haloperidol, fluphenazine, chlorpromazine hydrochloride, thioridazine hydrochloride, trifluoperazine hydrochloride, thiothixene hydrochloride. loxapine succinate, perphenazine, and molindone hydrochloride.

53. The method of claim 49, wherein the monoamine oxidase inhibitor is selected from the group consisting of phenelzine sulfate, tranylcypromine, and selegiline.

54. The method of claim 49, wherein the antiseizure drug is selected from the group consisting of oxcarbazepine, lamotrigene, topiramate, divalproex sodium, levetiracetam, gabapentin, tiagabine, and zonisamide.

55. A method for treating a patient for Bipolar II by administering an effective amount of a composition comprising one of the following combinations:
(a) lithium, a selective serotonin reuptake inhibitor, and an atypical neuroleptic;
(b) lithium, a selective serotonin reuptake inhibitor, and an antipsychotic drug;
(c) lithium, a selective serotonin reuptake inhibitor, and an antiseizure drug;
(d) lithium and an antipsychotic drug;
(e) lithium and monoamine oxidase inhibitor; and
(f) lithium, bupropion, and modafinil.

56. The method of claim 55, wherein the selective serotonin reuptake inhibitor is selected from the group consisting of citalopram, escitalopram, paroxetine, sertraline, fluoxetine, and duloxetine.

57. The method of claim 55, wherein the atypical neuroleptic is selected from the group consisting of aripiprazole, quetiapine fumarate, risperidone, ziprasidone, and olanzapine.

58. The method of claim 55, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

59. The method of claim 55, wherein the antiseizure drug is selected from the group consisting of oxcarbazepine, lamotrigene, topiramate, divalproex sodium, levetiracetam, gabapentin, tiagabine, and zonisamide.

60. The method of claim 55, wherein the monoamine oxidase inhibitor is selected from the group consisting of phenelzine sulfate, tranylcypromine, and selegiline.

61. A method for treating a patient for Borderline Personality Disorder by administering an effective amount of a composition comprising one of the following combinations:
(a) lithium, a selective serotonin reuptake inhibitor, and an atypical neuroleptic;
(b) lithium, a selective serotonin reuptake inhibitor, and an antipsychotic drug;
(c) lithium, a selective serotonin reuptake inhibitor and an antiseizure drug;
(d) bupropion and a tricyclic antidepressant;
(e) bupropion and a tetracyclic antidepressant;
(f) lithium and an antipsychotic drug;
(g) lithium and a monoamine oxidase inhibitor;
(h) lithium, bupropion, and modafinil;
(i) lithium, bupropion, and lamotrigine;
(j) bupropion and lamotrigine;
(k) lithium and bupropion; and
(l) lithium and mirtazapine.

62. The method of claim 61, wherein the selective serotonin reuptake inhibitor is selected from the group consisting of citalopram, escitalopram, paroxetine, sertraline, fluoxetine, and duloxetine.

63. The method of claim 61, wherein the atypical neuroleptic is selected from the group consisting of aripiprazole, quetiapine fumarate, risperidone, ziprasidone, and olanzapine.

64. The method of claim 61, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

65. The method of claim 61, wherein the antiseizure drug is selected from the group consisting of oxcarbazepine, lamotrigene, topiramate, divalproex sodium, levetiracetam, gabapentin, tiagabine, and zonisamide.

66. The method of claim 61, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

67. The method of claim 61, tricyclic antidepressant is selected from the group consisting of amitriptyline, clomipramine, doxepin, imipramine, trimipramine, desipramine, nortriptyline, and protriptyline.

68. The method of claim 61, wherein the tetracyclic antidepressant is selected from the group consisting of amoxapine and maprotiline.

69. The method of claim 61, wherein the monoamine oxidase inhibitor is selected from the group consisting of phenelzine sulfate, tranylcypromine, and selegiline.

70. A method for treating a patient for Cyclothymia by administering an effective amount of a composition comprising one of the following combinations:
(a) lithium, a selective serotonin reuptake inhibitor, and an atypical neuroleptic;
(b) lithium, a selective serotonin reuptake inhibitor, and an antipsychotic drug;
(c) lithium, a selective serotonin reuptake inhibitor and an antiseizure drug;
(d) bupropion and a tricyclic antidepressant;
(e) bupropion and a tetracyclic antidepressant;
(f) lithium and-an antipsychotic drug;
(g) lithium and a monoamine oxidase inhibitor;
(h) lithium, bupropion, and modafinil;
(i) lithium, bupropion, and lamotrigine;
(j) bupropion and lamotrigine;
(k) lithium and bupropion; and
(l) lithium and mirtazapine.

71. The method of claim 70, wherein the selective serotonin reuptake inhibitor is selected from the group consisting of citalopram, escitalopram, paroxetine, sertraline, fluoxetine, and duloxetine.

72. The method of claim 70, wherein the atypical neuroleptic is selected from the group consisting of aripiprazole, quetiapine fumarate, risperidone, ziprasidone, and olanzapine.

73. The method of claim 70, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

74. The method of claim 70, wherein the antiseizure drug is selected from the group consisting of oxcarbazepine, lamotrigene, topiramate, divalproex sodium, levetiracetam, gabapentin, tiagabine, and zonisamide.

75. The method of claim 70, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

76. The method of claim 70, wherein the tetracyclic antidepressant is selected from the group consisting of amoxapine and maprotiline.

77. The method of claim 70, wherein the monoamine oxidase inhibitor is selected from the group consisting of phenelzine sulfate, tranylcypromine, and selegiline.

78. A method for treating a patient for Depressive Personality Disorder by administering an effective amount of a composition comprising one of the following combinations:
(a) lithium, a selective serotonin reuptake inhibitor, and an antiseizure drug;
(b) lithium, a selective serotonin reuptake inhibitor, and bupropion
(c) bupropion and a tricyclic antidepressant;
(d) bupropion and a tetracyclic antidepressant;
(e) lithium and an antipsychotic drug;
(f) lithium and a monoamine oxidase inhibitor;
(g) lithium, bupropion, and modafinil;
(h) lithium, bupropion, and lamotrigine;
(i) bupropion and lamotrigine;
(j) lithium and bupropion; and
(k) lithium and mirtazapine.

79. The method of claim 78, wherein the selective serotonin reuptake inhibitor is selected from the group consisting of citalopram, escitalopram, paroxetine, sertraline, fluoxetine, and duloxetine.

80. The method of claim 78, wherein the antiseizure drug is selected from the group consisting of oxcarbazepine, lamotrigene, topiramate, divalproex sodium, levetiracetam, gabapentin, tiagabine, and zonisamide.

81. The method of claim 78, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

82. The method of claim 78, wherein the tetracyclic antidepressant is selected from the group consisting of amoxapine and maprotiline.

83. The method of claim 78, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

84. The method of claim 78, wherein the monoamine oxidase inhibitor is selected from the group consisting of phenelzine sulfate, tranylcypromine, and selegiline.

85. A method for treating a patient for Dysthymia by administering an effective amount of a composition comprising one of the following combinations:
(a) lithium, a selective serotonin reuptake inhibitor, and an antiseizure drug;
(b) lithium, a selective serotonin reuptake inhibitor, and an atypical neuroleptic;
(c) lithium, a selective serotonin reuptake inhibitor, and an antipsychotic drug
(d) lithium and an antipsychotic drug;
(e) lithium and bupropion;
(f) lithium, an antiseizure drug, and a tricyclic antidepressant;
(g) lithium and mirtazapine;
(h) lithium and a tricyclic antidepressant;
(i) lithium and a monoamine oxidase inhibitor;
(j) bupropion and a tricyclic antidepressant;
(k) bupropion and a tetracyclic antidepressant;
(l) lithium, bupropion, and modafinil;
(m) lithium, bupropion, and lamotrigine; and
(n) bupropion and lamotrigine.

86. The method of claim 85, wherein the selective serotonin reuptake inhibitor is selected from the group consisting of citalopram, escitalopram, paroxetine, sertraline, fluoxetine, and duloxetine.

87. The method of claim 85, wherein the antiseizure drug is selected from the group consisting of oxcarbazepine, lamotrigene, topiramate, divalproex sodium, levetiracetam, gabapentin, tiagabine, and zonisamide.

88. The method of claim 85, wherein the atypical neuroleptic is selected from the group consisting of aripiprazole, quetiapine fumarate, risperidone, ziprasidone, and olanzapine.

89. The method of claim 85, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

90. The method of claim 85, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

91. The method of claim 85, wherein the tetracyclic antidepressant is selected from the group consisting of amoxapine and maprotiline.

92. The method of claim 85, wherein the monoamine oxidase inhibitor is selected from the group consisting of phenelzine sulfate, tranylcypromine, and selegiline.

93. A method for treating a patient for Eating Disorder by administering an effective amount of a composition comprising one of the following combinations:
(a) lithium, a selective serotonin reuptake inhibitor, and an atypical neuroleptic;
(b) lithium, a selective serotonin reuptake inhibitor, an atypical neuroleptic, and an antiseizure drug;
(c) lithium and an antipsychotic drug; and
(d) lithium and a tricyclic antidepressant.

94. The method of claim 93, wherein the selective serotonin reuptake inhibitor is selected from the group consisting of citalopram, escitalopram, paroxetine, sertraline, fluoxetine, and duloxetine.

95. The method of claim 93, wherein the atypical neuroleptic is selected from the group consisting of aripiprazole, quetiapine fumarate, risperidone, ziprasidone, and olanzapine.

96. The method of claim 93, wherein the antiseizure drug is selected from the group consisting of oxcarbazepine, lamotrigene, topiramate, divalproex sodium, levetiracetam, gabapentin, tiagabine, and zonisamide.

97. The method of claim 93, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

98. The method of claim 93, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

99. A method for treating a patient for General Anxiety Disorder by administering an effective amount of a composition comprising one of the following combinations:
(a) lithium, a selective serotonin reuptake inhibitor, and an atypical neuroleptic; and
(b) lithium, a selective serotonin reuptake inhibitor, and an antipsychotic drug.

100. The method of claim 99, wherein the selective serotonin reuptake inhibitor is selected from the group consisting of citalopram, escitalopram, paroxetine, sertraline, fluoxetine, and duloxetine.

101. The method of claim 99, wherein the atypical neuroleptic is selected from the group consisting of aripiprazole, quetiapine fumarate, risperidone, ziprasidone, and olanzapine.

102. The method of claim 99, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

103. A method for treating a patient for Major Depressive Disorder by administering an effective amount of a composition comprising one of the following combinations:
(a) lithium and a tricyclic antidepressant;
(b) lithium and a monoamine oxidase inhibitor;
(c) lithium, a tricyclic antidepressant and an antiseizure drug;
(d) lithium and bupropion;
(e) lithium and mirtazapine;
(f) lithium, a selective serotonin reuptake inhibitor, and a tricyclic antidepressant;
(g) lithium, a selective serotonin reuptake inhibitor, a tricyclic antidepressant, and an atypical neuroleptic;
(h) lithium, a selective serotonin reuptake inhibitor, a tricyclic antidepressant, and an antiseizure drug;
(i) lithium, a selective serotonin reuptake inhibitor, a tricyclic antidepressant, an atypical neuroleptic and an antiseizure drug; and
(j) lithium, bupropion and modafinil.

104. The method of claim 103, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

105. The method of claim 103, wherein the monoamine oxidase inhibitor is selected from the group consisting of phenelzine sulfate, tranylcypromine, and selegiline.

106. The method of claim 103, wherein the antiseizure drug is selected from the group consisting of oxcarbazepine, lamotrigene, topiramate, divalproex sodium, levetiracetam, gabapentin, tiagabine, and zonisamide.

107. The method of claim 103, wherein the selective serotonin reuptake inhibitor is selected from the group consisting of citalopram, escitalopram, paroxetine, sertraline, fluoxetine, and duloxetine.

108. The method of claim 103, wherein the atypical neuroleptic is selected from the group consisting of aripiprazole, quetiapine fumarate, risperidone, ziprasidone, and olanzapine.

109. A method for treating a patient for Panic Disorder by administering an effective amount of a composition comprising lithium, a selective serotonin reuptake inhibitor, and an atypical neuroleptic.

110. The method of claim 109, wherein the selective serotonin reuptake inhibitor is selected from the group consisting of citalopram, escitalopram, paroxetine, sertraline, fluoxetine, and duloxetine.

111. The method of claim 109, wherein the atypical neuroleptic is selected from the group consisting of aripiprazole, quetiapine fumarate, risperidone, ziprasidone, and olanzapine.

112. A method for treating a patient for Posttraumatic Stress Disorder by administering an effective amount of a composition comprising lithium, a selective serotonin reuptake inhibitor, and an atypical neuroleptic.

113. The method of claim 112, wherein the selective serotonin reuptake inhibitor is selected from the group consisting of citalopram, escitalopram, paroxetine, sertraline, fluoxetine, and duloxetine.

114. The method of claim 112, wherein the atypical neuroleptic is selected from the group consisting of aripiprazole, quetiapine fumarate, risperidone, ziprasidone, and olanzapine.

115. A method for treating a patient for Pre-Menstrual Dysphoric Disorder by administering an effective amount of a composition comprising one of the following combinations:
(a) lithium, a selective serotonin reuptake inhibitor, and an antiseizure drug;
(b) lithium, a selective serotonin reuptake inhibitor, and an atypical neuroleptic; and
(c) lithium and bupropion.

116. The method of claim 1 5, wherein the selective serotonin reuptake inhibitor is selected from the group consisting of citalopram, escitalopram, paroxetine, sertraline, fluoxetine, and duloxetine.

117. The method of claim 115, wherein the antiseizure drug is selected from the group consisting of oxcarbazepine, lamotrigene, topiramate, divalproex sodium, levetiracetam, gabapentin, tiagabine, and zonisamide.

118. The method of claim 115 wherein the atypical neuroleptic is selected from the group consisting of aripiprazole, quetiapine fumarate, risperidone, ziprasidone, and olanzapine.

119. A method for treating a patient for Schizoaffective Disorder, depressed type, by administering an effective amount of a composition comprising one of the following combinations:
(a) lithium and an antipsychotic drug;
(b) lithium, a selective serotonin reuptake inhibitor, and an atypical neuroleptic;
(c) lithium, a selective serotonin reuptake inhibitor, and an antipsychotic drug;
(d) lithium, a selective serotonin reuptake inhibitor, an antipsychotic drug, and an antiseizure drug;
(e) lithium, a selective serotonin reuptake inhibitor, an atypical neuroleptic, and an antiseizure drug;
(f) lithium, a selective serotonin reuptake inhibitor, and bupropion;
(g) lithium, a selective serotonin reuptake inhibitor, bupropion and an atypical neuroleptic; and
(h) lithium, a selective serotonin reuptake inhibitor, bupropion and an antipsychotic drug.

120. The method of claim 119, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

121. The method of claim 119, wherein the atypical neuroleptic is selected from the group consisting of aripiprazole, quetiapine fumarate, risperidone, ziprasidone, and olanzapine.

122. The method of claim 119, wherein the selective serotonin reuptake inhibitor is selected from the group consisting of citalopram, escitalopram, paroxetine, sertraline, fluoxetine, and duloxetine.

123. The method of claim 119, wherein the antiseizure drug is selected from the group consisting of oxcarbazepine, lamotrigene, topiramate, divalproex sodium, levetiracetam, gabapentin, tiagabine, and zonisamide.

124. A method for treating a patient for Schizoaffective Disorder, manic type, by administering an effective amount of a composition comprising one of the following combinations:
(a) lithium and an antipsychotic drug;
(b) lithium, an atypical neuroleptic, and an antiseizure drug;
(c) lithium, an antipsychotic drug and an antiseizure drug;
(d) lithium, bupropion and an atypical neuroleptic;
(e) lithium bupropion and an antipsychotic drug;
(f) lithium, bupropion, an atypical neuroleptic and an antiseizure drug;
(g) lithium, bupropion, an antipsychotic drug and an antiseizure drug;
(h) lithium, a selective serotonin reuptake inhibitor, and an atypical neuroleptic;
(i) lithium, a selective serotonin reuptake inhibitor, and an antipsychotic drug;
(j) lithium, a selective serotonin reuptake inhibitor, and an antiseizure drug;
(k) lithium, a selective serotonin reuptake inhibitor, an atypical neuroleptic and an antipsychotic drug; and
(l) bupropion and an antiseizure drug.

125. The method of claim 124, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

126. The method of claim 124, wherein the atypical neuroleptic is selected from the group consisting of aripiprazole, quetiapine fumarate, risperidone, ziprasidone, and olanzapine.

127. The method of claim 124, wherein the antiseizure drug is selected from the group consisting of oxcarbazepine, lamotrigene, topiramate, divalproex sodium, levetiracetam, gabapentin, tiagabine, and zonisamide.

128. The method of claim 124, wherein the selective serotonin reuptake inhibitor is selected from the group consisting of citalopram, escitalopram, paroxetine, sertraline, fluoxetine, and duloxetine.

129. A method for treating a patient for Schizoaffective Disorder, mixed type, by administering an effective amount of a composition comprising one of the following combinations:
(a) bupropion and an antiseizure drug;
(b) lithium, and an antipsychotic drug;
(c) lithium, an atypical neuroleptic, and an antiseizure drug;
(d) lithium, an antipsychotic drug, and an antiseizure drug;
(e) lithium, bupropion, and an atypical neuroleptic;
(f) lithium, bupropion, and an antipsychotic drug;
(g) lithium, bupropion, an atypical neuroleptic, and an antiseizure drug;
(h) lithium, bupropion, an antipsychotic drug, and an antiseizure drug;
(i) lithium, a selective serotonin reuptake inhibitor, and an atypical neuroleptic;
(j) lithium, a selective serotonin reuptake inhibitor, and an antipsychotic drug;
(k) lithium, a selective serotonin reuptake inhibitor, an atypical neuroleptic, and an antiseizure drug; and
(l) lithium, a selective serotonin reuptake inhibitor, an antipsychotic drug, and antiseizure drug.

130. The method of claim 129, wherein the antiseizure drug is selected from the group consisting of oxcarbazepine, lamotrigene, topiramate, divalproex sodium, levetiracetam, gabapentin, tiagabine, and zonisamide.

131. The method of claim 129, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

132. The method of claim 129, wherein the atypical neuroleptic is selected from the group consisting of aripiprazole, quetiapine fumarate, risperidone, ziprasidone, and olanzapine.

133. The method of claim 129, wherein the selective serotonin reuptake inhibitor is selected from the group consisting of citalopram, escitalopram, paroxetine, sertraline, fluoxetine, and duloxetine.

134. A method for treating a patient for Seasonal Affective Disorder by administering an effective amount of a composition comprising one of the following combinations:
(a) lithium, a selective serotonin reuptake inhibitor, and an atypical neuroleptic;
(b) lithium, a selective serotonin reuptake inhibitor, and an antiseizure drug;
(c) lithium and bupropion;
(d) lithium, bupropion and an atypical neuroleptic; and
(e) lithium and an tricyclic antidepressant.

135. The method of claim 134, wherein the selective serotonin reuptake inhibitor is selected from the group consisting of citalopram, escitalopram, paroxetine, sertraline, fluoxetine, and duloxetine.

136. The method of claim 134, wherein the atypical neuroleptic is selected from the group consisting of aripiprazole, quetiapine fumarate, risperidone, ziprasidone, and olanzapine.

137. The method of claim 134, wherein the antiseizure drug is selected from the group consisting of oxcarbazepine, lamotrigene, topiramate, divalproex sodium, levetiracetam, gabapentin, tiagabine, and zonisamide.

138. The method of claim 134, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

139. The method of claim 134, wherein the selective serotonin reuptake inhibitor is selected from the group consisting of citalopram, escitalopram, paroxetine, sertraline, fluoxetine, and duloxetine.

140. A method for treating a patient for Social Anxiety Disorder by administering an effective amount of a composition comprising one of the following combinations:
(a) lithium, a selective serotonin reuptake inhibitor, and an atypical neuroleptic; and
(b) lithium, a selective serotonin reuptake inhibitor, and an antipsychotic drug.

141. The method of claim 140, wherein the selective serotonin reuptake inhibitor is selected from the group consisting of citalopram, escitalopram, paroxetine, sertraline, fluoxetine, and duloxetine.

142. The method of claim 140, wherein the atypical neuroleptic is selected from the group consisting of aripiprazole, quetiapine fumarate, risperidone, ziprasidone, and olanzapine.

143. The method of claim 140, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

144. A method for treating a patient for Substance Use Disorder by administering an effective amount of a composition comprising one of the following combinations:
(a) lithium and bupropion;
(b) lithium, bupropion, and an atypical neuroleptic;
(c) lithium, bupropion and an antipsychotic drug;
(d) lithium, bupropion, and an antiseizure drug;
(e) lithium, a selective serotonin reuptake inhibitor, and an antipsychotic drug;
(f) lithium, a selective serotonin reuptake inhibitor, and an antiseizure drug;
(g) lithium, a selective serotonin reuptake inhibitor, and an atypical neuroleptic;
(h) lithium, a selective serotonin reuptake inhibitor, an antiseizure drug, and an atypical neuroleptic;
(i) lithium, a selective serotonin reuptake inhibitor, and a tricyclic antidepressant;
(j) lithium and a monoamine oxidase inhibitor;
(k) bupropion and a tricyclic antidepressant;
(l) bupropion and a tetracyclic antidepressant;
(m) lithium and an antipsychotic drug;
(n) lithium, bupropion, and modafinil;
(o) lithium, bupropion, and lamotrigine;
(p) bupropion and lamotrigine; and
(q) lithium and mirtazapine.

145. The method of claim 144, wherein the atypical neuroleptic is selected from the group consisting of aripiprazole, quetiapine fumarate, risperidone, ziprasidone, and olanzapine.

146. The method of claim 144, wherein the antipsychotic drug is selected from the group consisting of haloperidol, fluphenazine, chlorpromazine hydrochloride, thioridazine hydrochloride, trifluoperazine hydrochloride, thiothixene hydrochloride, loxapine succinate, perphenazine, and molindone hydrochloride.

147. The method of claim 144, wherein the antiseizure drug is selected from the group consisting of oxcarbazepine, lamotrigene, topiramate, divalproex sodium, levetiracetam, gabapentin, tiagabine, and zonisamide.

148. The method of claim 144, wherein the selective serotonin reuptake inhibitor is selected from the group consisting of citalopram, escitalopram, paroxetine, sertraline, fluoxetine, and duloxetine.

149. The method of claim 144, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

150. The method of claim 144, wherein the tetracyclic antidepressant is selected from the group consisting of amoxapine and maprotiline.

151. The method of claim 144, wherein the monoamine oxidase inhibitor is selected from the group consisting of phenelzine sulfate, tranylcypromine, and selegiline.

152. A method for treating a patient for Treatment Resistant Depression by administering an effective amount of a composition comprising one of the following combinations:
(a) lithium, at selective serotonin reuptake inhibitor, and an atypical neuroleptic;
(b) lithium, a selective serotonin reuptake inhibitor, and an antiseizure drug;
(c) lithium, a selective serotonin reuptake inhibitor, and an tricyclic antidepressant;
(d) lithium and bupropion;
(e) lithium, bupropion and an atypical neuroleptic;
(f) lithium, bupropion, and an antiseizure drug;
(g) lithium and a monoamine oxidase inhibitor;
(h) lithium and a tricyclic antidepressant;
(i) lithium, a tricyclic antidepressant, and an atypical neuroleptic;
(j) lithium, a tricyclic antidepressant, an atypical neuroleptic, and an antiseizure drug;
(k) lithium, a selective serotonin reuptake inhibitor and venlafaxine;
(l) lithium, a selective serotonin reuptake inhibitor, and bupropion;
(m) lithium, a selective serotonin reuptake inhibitor, venlafaxine and an antiseizure drug;
(n) lithium, a selective serotonin reuptake inhibitor, bupropion and an antiseizure drug;
(o) bupropion and a tricyclic antidepressant;
(p) bupropion and a tetracyclic antidepressant;
(q) lithium and antipsychotic drug;
(r) lithium, bupropion, and modafinil;
(s) lithium, bupropion, and lamotrigine;
(t) bupropion and lamotrigine; and
(u) lithium and mirtazapine.

153. The method of claim 152, wherein the selective serotonin reuptake inhibitor is selected from the group consisting of citalopram, escitalopram, paroxetine, sertraline, fluoxetine, and duloxetine.

154. The method of claim 152, wherein the atypical neuroleptic is selected from the group consisting of aripiprazole, quetiapine fumarate, risperidone, ziprasidone, and olanzapine.

155. The method of claim 152, wherein the antiseizure drug is selected from the group consisting of oxcarbazepine, lamotrigene, topiramate, divalproex sodium, levetiracetam, gabapentin, tiagabine, and zonisamide.

156. The method of claim 152, wherein the tricyclic antidepressant is selected from the group consisting of desipramine, nortriptyline, amitriptyline hydrochloride, clomipramine hydrochloride, doxepin hydrochloride, imipramine pamoate, trimipramine maleate, and protriptyline.

157. The method of claim 152, wherein the monoamine oxidase inhibitor is selected from the group consisting of phenelzine sulfate, tranylcypromine, and selegiline.

158. The method of claim 152, wherein the tetracyclic antidepressant is selected from the group consisting of amoxapine and maprotiline.
